(19) United States
(12) Patent Application Publication
(10) Pub. No.: US 2024/0091203 A1
(43) Pub. Date: Mar. 21, 2024

(54) METHODS FOR TREATING NON-OBSTRUCTIVE HYPERTROPHIC CARDIOMYOPATHY

(71) Applicant: Cytokinetics, Incorporated, South San Francisco, CA (US)

(72) Inventors: Stephen B. HEITNER, Portland, OR (US); Fady MALIK, Burlingame, CA (US); Stuart KUPFER, Highland Park, IL (US); Daniel Louis JACOBY, Madison, CT (US)

(21) Appl. No.: 18/355,195

(22) Filed: Jul. 19, 2023

Related U.S. Application Data
(60) Provisional application No. 63/501,088, filed on May 9, 2023.
(60) Provisional application No. 63/495,966, filed on Apr. 13, 2023.
(60) Provisional application No. 63/491,010, filed on Mar. 17, 2023.
(60) Provisional application No. 63/486,594, filed on Feb. 23, 2023.
(60) Provisional application No. 63/375,026, filed on Sep. 8, 2022.
(60) Provisional application No. 63/368,967, filed on Jul. 20, 2022.

(51) Int. Cl. A61K 31/4245; A61P 9/10

(52) U.S. Cl. CPC A61K 31/4245; A61P 9/10; A61B 8/0883

(57) ABSTRACT

Methods for treating non-obstructive hypertrophic cardiomyopathy or hypertrophic cardiomyopathy with mid-ventricular obstruction (MVO) are described herein. The treatment methods include the administration of a cardiac myosin inhibitor (CK-3773274, also referred to as CK-274 or aficamten) and may include titrating an administrated daily dose based on a component of an echocardiogram. The daily dose may be increased, maintained, or decreased, or terminated, based on the echocardiogram.

### CROSS-REFERENCE TO RELATED APPLICATIONS

This application claims priority to U.S. Provisional Application No. 63/368,967, filed Jul. 20, 2022; U.S. Provisional Application No. 63/375,026, filed Sep. 8, 2022; U.S. Provisional Application No. 63/486,594, filed Feb. 23, 2023; U.S. Provisional Application No. 63/491,010, filed Mar. 17, 2023; U.S. Provisional Application No. 63/495,966, filed Apr. 13, 2023; and U.S. Provisional Application No. 63/501,088, filed May 9, 2023; the contents of which are hereby incorporated by reference in their entirety for all purposes.

### FIELD

The disclosure herein relates to the treatment of non-obstructive hypertrophic cardiomyopathy or hypertrophic cardiomyopathy with mid-ventricular obstruction, and compounds and compositions that may be used for treating non-obstructive hypertrophic cardiomyopathy.

### BACKGROUND

Hypertrophic cardiomyopathy (HCM) is a disease in which the heart muscle (myocardium) becomes abnormally thick (hypertrophied). The thickening of cardiac muscle leads to the inside of the left ventricle becoming smaller and stiffer, and thus the ventricle becomes less able to relax and fill with blood. 30% of patients with HCM do not have left ventricular outflow tract (LVOT) obstruction, either at rest or with physiologic provocation (i.e. Valsalva maneuver or exercise); these patients are categorized as having non-obstructive HCM (nHCM). Patients with nHCM have a similar risk of developing heart failure and all-cause mortality compared to patients with resting oHCM but experience a higher burden of life-threatening ventricular arrhythmias (sustained ventricular tachycardia or ventricular fibrillation) compared to patients with resting or latent obstruction. Thus, there is a need for therapies that address this condition.

### BRIEF SUMMARY

Methods and compositions for treating non-obstructive hypertrophic cardiomyopathy are described herein. A cardiac myosin inhibitor (CK-3773274, also referred to as CK-274 or aficamten), or a pharmaceutically acceptable salt thereof, can be used to treat non-obstructive hypertrophic cardiomyopathy or hypertrophic cardiomyopathy with mid-ventricular obstruction. As further described herein, the daily dose of CK-274 may be titrated based on the results of an echocardiogram.

A method of treating non-obstructive hypertrophic cardiomyopathy (nHCM) or HCM with mid-ventricular obstruction (MVO, also known as “mid-cavitary obstruction”) in a patient in need thereof can include administering to the patient a therapeutically effective amount of CK-274, or a pharmaceutically acceptable salt thereof, wherein the therapeutically effective amount of CK-274, or a pharmaceutically acceptable salt thereof, is selected by titrating a daily dose of CK-274, or a pharmaceutically acceptable salt thereof, administered to the patient. In some embodiments, the dose is titrated once during a course of treatment. In some embodiments, the dose is titrated two or more times during a course of treatment. The daily dose may be administered to a patient at a constant amount for about two weeks before the daily dose amount is titrated.

In some implementations of the above methods, CK-274, or a pharmaceutically acceptable salt thereof, is administered at a daily dose of about 5 mg to about 20 mg. In some embodiments, the daily dose is about 5 mg. In some embodiments, the daily dose is about 10 mg. In some embodiments, the daily dose is about 15 mg. In some embodiments, the daily dose is about 20 mg. As appreciated by those skilled in the art and described herein, unless indicated otherwise, amount, e.g., in a dose, is of the amount of CK-274 free base or the corresponding amount of CK-274 free base when a non-free base form, e.g., a pharmaceutically acceptable salt, is administered.

In some implementations, the daily dose is administered as a single dose each day. In some implementations, the daily dose is administered in 2 divided doses.

In some embodiments, as described herein, a method, e.g., a method of treating non-obstructive hypertrophic cardiomyopathy (nHCM) or HCM with mid-ventricular obstruction (MVO) in a patient in need thereof, or a method for reducing angina frequency, in a patient in need thereof, comprises administering to the patient a first daily dose of CK-274, or a pharmaceutically acceptable salt thereof, for a first time period, and a second daily dose of CK-274, or a pharmaceutically acceptable salt thereof, for a second time period. In some embodiments, a first daily dose and a second daily dose are different. In some embodiments, a second daily dose is lower than the first daily dose. In some embodiments, a second daily dose is higher than the first daily dose. In some embodiments, a second daily dose is the same as the first daily dose. In some embodiments, a method further comprises administering a third daily dose for a third time period as described herein. In some embodiments, a method further comprises administering a fourth daily dose for a fourth time period as described herein. Various technologies and criteria, e.g., biplane LVEF component of an echocardiogram after a daily dose for a time period, may be utilized to select a next daily dose. In some embodiments, a daily dose is of about 15 mg of CK-274 (e.g., as a third or a fourth daily dose).

In some embodiments, a method of treating non-obstructive hypertrophic cardiomyopathy (nHCM) or HCM with mid-ventricular obstruction (MVO) in a patient in need thereof comprises: administering to the patient a first daily dose of CK-274, or a pharmaceutically acceptable salt thereof, for a first time period; and based on the biplane LVEF component of a first echocardiogram for the patient acquired after the first time period, administering to the patient a second daily dose of CK-274, or a pharmaceutically acceptable salt thereof, for a second time period or terminating the administering of CK-274, or a pharmaceutically acceptable salt thereof, to the patient. The method may include selecting the second daily dose of CK-274, or a pharmaceutically acceptable salt thereof, based on the biplane LVEF component of the first echocardiogram. In some embodiments of the methods described herein, the patient undergoes two or more echocardiograms within the first time period, and the second daily dose is selected based on the combined results of two or more echocardiograms obtained within the first time period.

In some implementations of the above methods, the administering of CK-274, or a pharmaceutically acceptable salt thereof, to the patient is terminated or interrupted when the biplane LVEF of the first echocardiogram is below a predetermined biplane LVEF threshold. For example, the predetermined biplane LVEF threshold may be 50%. For example, the predetermined biplane LVEF threshold may be 40%.

In some implementations of the above methods, the second daily dose of CK-274, or a pharmaceutically acceptable salt thereof, is lower than the first daily dose of CK-274, or a pharmaceutically acceptable salt thereof, when the biplane LVEF of the first echocardiogram is below a predetermined biplane LVEF threshold. For example, the predetermined biplane LVEF threshold may be 50%.

In some implementations of the above methods, the second daily dose of CK-274, or a pharmaceutically acceptable salt thereof, is the same as the first daily dose of CK-274, or a pharmaceutically acceptable salt thereof, when the biplane LVEF of the first echocardiogram is within the predetermined biplane LVEF thresholds. In some embodiments, the predetermined biplane LVEF thresholds are: biplane LVEF ≥50% and biplane LVEF <55%. In some embodiments, the predetermined biplane LVEF thresholds are: biplane LVEF ≥50% and biplane LVEF <60%.

In some implementations of the above methods, the second daily dose of CK-274, or a pharmaceutically acceptable salt thereof, is greater than the first daily dose of CK-274, or a pharmaceutically acceptable salt thereof, when the biplane LVEF of the first echocardiogram is at or above the predetermined biplane LVEF threshold. In some embodiments, the predetermined biplane LVEF threshold is 55%. In some embodiments, the predetermined biplane LVEF threshold is 60%.

In some implementations of the above methods, the first daily dose of CK-274, or a pharmaceutically acceptable salt thereof, is about 5 mg of CK-274. In some embodiments, the second daily dose of CK-274, or a pharmaceutically acceptable salt thereof, is about 5 mg or about 10 mg of CK-274.

In some implementations of the above methods, first time period is about 2 weeks. In some embodiments, the second time period is about 2 weeks.

In some implementations of the above methods, the second daily dose of CK-274, or a pharmaceutically acceptable salt thereof, is administered to the patient for the second time period, and the method further comprises, based on the biplane LVEF of a second echocardiogram for the patient acquired after the second time period and the second daily dose of CK-274, or a pharmaceutically acceptable salt thereof, administering to the patient a third daily dose of CK-274, or a pharmaceutically acceptable salt thereof for a third time period or terminating the administering of CK-274, or a pharmaceutically acceptable salt thereof to the patient. In some embodiments, the method comprises selecting the third daily dose of CK-274, or a pharmaceutically acceptable salt thereof, based on the biplane LVEF of the second echocardiogram and the second daily dose. In some embodiments of the methods described herein, the patient undergoes two or more echocardiograms within the second time period, and the third daily dose is selected based on the combined results of two or more echocardiograms obtained within the second time period.

In some embodiments of the above methods, the third daily dose of CK-274, or a pharmaceutically acceptable salt thereof, is lower than the second daily dose of CK-274, or a pharmaceutically acceptable salt thereof, or the administering of CK-274, or a pharmaceutically acceptable salt thereof, to the patient is terminated, when the biplane LVEF of the second echocardiogram is below the predetermined biplane LVEF threshold. In some embodiments, the predetermined biplane LVEF threshold is 50%.

In some embodiments of the above methods, the administering of CK-274, or a pharmaceutically acceptable salt thereof, to the patient is terminated when the biplane LVEF of the second echocardiogram is below the predetermined biplane LVEF threshold, and the second daily dose of CK-274, or a pharmaceutically acceptable salt thereof, is the same as the first daily dose of CK-274, or a pharmaceutically acceptable salt thereof. In some embodiments, the predetermined biplane LVEF threshold is 50%. For example, if a method comprises treating the patient with a first daily dose of about 5 mg of CK-274 and a second daily dose of about 5 mg of CK-274, and if the biplane LVEF of the second echocardiogram is below the biplane LVEF threshold of 50%, then the administering of CK-274, or a pharmaceutically acceptable salt thereof, to the patient is terminated.

In some embodiments of the above methods, the third daily dose of CK-274, or a pharmaceutically acceptable salt thereof, is the same as the first daily dose of CK-274, or a pharmaceutically acceptable salt thereof, when the second daily dose of CK-274, or a pharmaceutically acceptable salt thereof, is higher than the first daily dose of CK-274, or a pharmaceutically acceptable salt thereof, and the biplane LVEF of the second echocardiogram is below the predetermined biplane LVEF threshold. In some embodiments, the predetermined biplane LVEF threshold is 50%. For example, if a method comprises treating the patient with a first daily dose of about 5 mg of CK-274 and a second daily dose of about 10 mg of CK-274, and if the biplane LVEF of the second echocardiogram is below the biplane LVEF threshold of 50%, then the third daily dose of CK-274 is returned to 5 mg of CK-274.

In some embodiments of the above methods, the third daily dose of CK-274, or a pharmaceutically acceptable salt thereof, is the same as the second daily dose of CK-274, or a pharmaceutically acceptable salt thereof, when the biplane LVEF of the second echocardiogram is within the predetermined biplane LVEF thresholds. In some embodiments, the predetermined biplane LVEF thresholds are: biplane LVEF ≥50% and biplane LVEF <55%. In some embodiments, the predetermined biplane LVEF thresholds are: biplane LVEF ≥50% and biplane LVEF <60%.

In some embodiments of the above methods, the third daily dose of CK-274, or a pharmaceutically acceptable salt thereof, is greater than the second daily dose of CK-274, or a pharmaceutically acceptable salt thereof, when the biplane LVEF of the second echocardiogram is at or above the predetermined biplane LVEF threshold. In some embodiments, the predetermined biplane LVEF threshold is 55%. In some embodiments, the predetermined biplane LVEF threshold is 60%.

In some embodiments of the above methods, the first daily dose of CK-274, or a pharmaceutically acceptable salt thereof, is about 5 mg of CK-274, the second daily dose of CK-274, or a pharmaceutically acceptable salt thereof, is about 5 mg or about 10 mg of CK-274, and the third daily dose of CK-274, or a pharmaceutically acceptable salt thereof, is about 5 mg, about 10 mg, or about 15 mg of CK-274.

In some embodiments of the above methods, the method further comprises measuring the component of the second echocardiogram.

In some embodiments of the above methods, the third time period is about 2 weeks.

In some embodiments of the above methods, the third daily dose of CK-274, or a pharmaceutically acceptable salt thereof, is administered to the patient for the third time period, the method further comprising, based on a component of a third echocardiogram for the patient acquired after the third time period and the third daily dose of Compound, or a pharmaceutically acceptable salt thereof, administering to the patient a fourth daily dose of CK-274, or a pharmaceutically acceptable salt thereof, for a fourth time period or terminating the administering of CK-274, or a pharmaceutically acceptable salt thereof, to the patient. In some embodiments, the method further comprises selecting the fourth daily dose of CK-274, or a pharmaceutically acceptable salt thereof, based on the biplane LVEF of the third echocardiogram and the third daily dose. In some embodiments of the methods described herein, the patient undergoes two or more echocardiograms within the third time period, and the fourth daily dose is selected based on the combined results of two or more echocardiograms obtained within the third time period.

In some embodiments of the above methods, the fourth daily dose of CK-274, or a pharmaceutically acceptable salt thereof, is lower than the third daily dose of CK-274, or a pharmaceutically acceptable salt thereof, or the administering of CK-274, or a pharmaceutically acceptable salt thereof, to the patient is terminated, when the biplane LVEF of the third echocardiogram is below the predetermined biplane LVEF threshold.

In some embodiments, the administering of CK-274, or a pharmaceutically acceptable salt thereof, to the patient is terminated when the biplane LVEF of the third echocardiogram is below the predetermined biplane LVEF threshold and the third daily dose of CK-274, or a pharmaceutically acceptable salt thereof, is the same as the first daily dose of CK-274, or a pharmaceutically acceptable salt thereof, or lower. In some embodiments, the predetermined biplane LVEF threshold is 50%.

In some embodiments of the above methods, the fourth daily dose of CK-274, or a pharmaceutically acceptable salt thereof, is the same as the second daily dose of CK-274, or a pharmaceutically acceptable salt thereof, when the third daily dose of CK-274, or a pharmaceutically acceptable salt thereof, is higher than the second daily dose of CK-274, or a pharmaceutically acceptable salt thereof, and the biplane LVEF of the third echocardiogram is at or above the predetermined biplane LVEF threshold; or, the fourth daily dose of CK-274, or a pharmaceutically acceptable salt thereof, is the same as the first daily dose of CK-274, or a pharmaceutically acceptable salt thereof, when the third daily dose of CK-274, or a pharmaceutically acceptable salt thereof, is the same as the second daily dose of CK-274, or a pharmaceutically acceptable salt thereof, and the biplane LVEF of the third echocardiogram is at or above the predetermined biplane LVEF threshold. In some embodiments, the predetermined biplane LVEF threshold is 50%.

In some embodiments of the above methods, the fourth daily dose of CK-274, or a pharmaceutically acceptable salt thereof, is greater than the third daily dose of CK-274, or a pharmaceutically acceptable salt thereof, when the biplane LVEF of the third echocardiogram is at or above the predetermined biplane LVEF threshold. In some embodiments, the predetermined biplane LVEF threshold is 55%. In some embodiments, the predetermined biplane LVEF threshold is 60%.

In some embodiments of the above methods, the first daily dose of CK-274, or a pharmaceutically acceptable salt thereof, is about 5 mg of CK-274, the second daily dose of CK-274, or a pharmaceutically acceptable salt thereof, is about 5 mg or about 10 mg of CK-274, the third daily dose of CK-274, or a pharmaceutically acceptable salt thereof, is about 5 mg, about 10 mg, or about 15 mg of CK-274, and the fourth daily dose of CK-274, or a pharmaceutically acceptable salt thereof, is about 5 mg, about 10 mg, or about 15 mg of CK-274.

In some embodiments, a daily dose is about 15 mg of CK-274. In some embodiments, a third daily dose is about 15 mg of CK-274. In some embodiments, a fourth daily dose is about 15 mg of CK-274. In some embodiments, a daily dose is about 20 mg of CK-274. In some embodiments, a fourth daily dose is about 20 mg of CK-274. In some embodiments, a daily dose is administered as a tablet. In some embodiments, amount of CK-274, which may exist in multiple forms (e.g., free form, pharmaceutically acceptable salt forms, polymorph forms, etc.), in a tablet is about a daily dose as described herein. In some embodiments, the amount of CK-274 in a tablet is about 5 mg. In some embodiments, the amount of CK-274 in a tablet is about 10 mg. In some embodiments, the amount of CK-274 in a tablet is about 15 mg. In some embodiments, the amount of CK-274 in a tablet is about 20 mg. In some embodiments, the amount of CK-274 in a tablet is about half of a daily dose as described herein. In some embodiments, the amount of CK-274 in a tablet is about 2.5 mg. In some embodiments, the amount of CK-274 in a tablet is about 5 mg. In some embodiments, the amount of CK-274 in a tablet is about 7.5 mg. In some embodiments, the amount of CK-274 in a tablet is about 10 mg.

In some embodiments of the above methods, the first daily dose of CK-274, or a pharmaceutically acceptable salt thereof, is about 5 mg of CK-274, the second daily dose of CK-274, or a pharmaceutically acceptable salt thereof, is about 5 mg or about 10 mg of CK-274, the third daily dose of CK-274, or a pharmaceutically acceptable salt thereof, is about 5 mg, about 10 mg, or about 15 mg of CK-274, and the fourth daily dose of CK-274, or a pharmaceutically acceptable salt thereof, is about 5 mg, about 10 mg, about 15 mg, or about 20 mg of CK-274.

In some embodiments of the above methods, the method further comprises measuring the biplane LVEF of the third echocardiogram.

In some embodiments of the above methods, the fourth time period is about 2 weeks.

In some implementations, a method of treating non-obstructive hypertrophic cardiomyopathy (nHCM) or HCM with mid-ventricular obstruction (MVO) in a patient in need thereof, comprises: administering to the patient a first daily dose of CK-274, or a pharmaceutically acceptable salt thereof, for a first time period; and based a first echocardiogram comprising a biplane LVEF for the patient acquired after the first time period, administering to the patient a second daily dose of CK-274, or a pharmaceutically acceptable salt thereof, for a second time period or terminating the administering of CK-274 to the patient, wherein: the administering of CK-274, or a pharmaceutically acceptable salt thereof, to the patient is terminated if the biplane LVEF of the first echocardiogram is below a first predetermined biplane LVEF threshold; the second daily dose of CK-274, or a pharmaceutically acceptable salt thereof, is the same as the first daily dose of CK-274, or a pharmaceutically acceptable salt thereof, if the biplane LVEF is at or above the first predetermined biplane LVEF threshold and below a second predetermined biplane LVEF threshold; and the second daily dose of CK-274, or a pharmaceutically acceptable salt thereof, is greater than the first daily dose of CK-274, or a pharmaceutically acceptable salt thereof, if the biplane LVEF of the first echocardiogram is at or above the second predetermined biplane LVEF threshold.

In some embodiments of the above methods, the first daily dose of CK-274, or a pharmaceutically acceptable salt thereof, is about 5 mg of CK-274 and the second daily dose of CK-274, or a pharmaceutically acceptable salt thereof, is about 5 mg or about 10 mg of CK-274.

In some embodiments of the above methods, the method further comprises measuring the biplane LVEF for the first echocardiogram.

In some embodiments of the above methods, the first time period is about 2 weeks.

In some embodiments of the above methods, the second time period is about 2 weeks.

In some embodiments of the above methods, the second daily dose of CK-274, or a pharmaceutically acceptable salt thereof, is administered to the patient for the second time period, the method further comprising, based a second echocardiogram comprising a biplane LVEF for the patient acquired after the second time period and the second daily dose of CK-274, or a pharmaceutically acceptable salt thereof, administering to the patient a third daily dose of CK-274, or a pharmaceutically acceptable salt thereof, for a third time period or terminating the administering of CK-274, or a pharmaceutically acceptable salt thereof, to the patient, wherein: the administering of CK-274, or a pharmaceutically acceptable salt thereof, to the patient is terminated if the biplane LVEF of the second echocardiogram is below the first predetermined biplane LVEF threshold and the second daily dose of CK-274, or a pharmaceutically acceptable salt thereof, is the same as the first daily dose of CK-274, or a pharmaceutically acceptable salt thereof; the third daily dose of CK-274, or a pharmaceutically acceptable salt thereof, is lower than the second daily dose of CK-274, or a pharmaceutically acceptable salt thereof, if the biplane LVEF of the second echocardiogram is below the first predetermined biplane LVEF threshold and the second daily dose of CK-274, or a pharmaceutically acceptable salt thereof, is higher than the first daily dose of CK-274, or a pharmaceutically acceptable salt thereof; the third daily dose of CK-274, or a pharmaceutically acceptable salt thereof, is the same as the second daily dose of CK-274, or a pharmaceutically acceptable salt thereof, if the biplane LVEF is at or above the first predetermined biplane LVEF threshold and below the second predetermined biplane LVEF threshold; and the third daily dose of CK-274, or a pharmaceutically acceptable salt thereof, is greater than the second daily dose of CK-274, or a pharmaceutically acceptable salt thereof, if the biplane LVEF of the second echocardiogram is above the second predetermined biplane LVEF threshold.

In some embodiments of the above methods, the first daily dose of CK-274, or a pharmaceutically acceptable salt thereof, is about 5 mg of CK-274, the second daily dose of CK-274, or a pharmaceutically acceptable salt thereof, is about 5 mg or about 10 mg of CK-274, and the third daily dose of CK-274, or a pharmaceutically acceptable salt thereof, is about 5 mg, about 10 mg, or about 15 mg of CK-274.

In some embodiments of the above methods, the method further comprises measuring the biplane LVEF for the second echocardiogram.

In some embodiments of the above methods, the third time period is about 2 weeks.

In some embodiments of the above methods, the third daily dose of CK-274, or a pharmaceutically acceptable salt thereof, is administered to the patient for the third time period, the method further comprising, based on a third echocardiogram comprising a biplane LVEF the patient acquired after the third time period and the third dose of CK-274, or a pharmaceutically acceptable salt thereof, administering to the patient a fourth daily dose of CK-274, or a pharmaceutically acceptable salt thereof, for a fourth time period or terminating the administering of CK-274 to the patient, wherein: the administering of CK-274, or a pharmaceutically acceptable salt thereof, to the patient is terminated if the biplane LVEF of the third echocardiogram is below the first predetermined biplane LVEF threshold and the third daily dose of CK-274, or a pharmaceutically acceptable salt thereof, is the same as the first daily dose of CK-274, or a pharmaceutically acceptable salt thereof; the fourth daily dose of CK-274, or a pharmaceutically acceptable salt thereof, is lower than the third daily dose of CK-274, or a pharmaceutically acceptable salt thereof, if the biplane LVEF of the third echocardiogram is below the first predetermined biplane LVEF threshold and the third daily dose of CK-274, or a pharmaceutically acceptable salt thereof, is higher than the first daily dose of CK-274, or a pharmaceutically acceptable salt thereof; the fourth daily dose of CK-274, or a pharmaceutically acceptable salt thereof, is the same as the third daily dose of CK-274, or a pharmaceutically acceptable salt thereof, if the biplane LVEF is at or above the first predetermined biplane LVEF threshold and below the second predetermined biplane LVEF threshold; and the fourth daily dose of CK-274, or a pharmaceutically acceptable salt thereof, is greater than the third daily dose of CK-274, or a pharmaceutically acceptable salt thereof, if the biplane LVEF of the third echocardiogram is at or above the second predetermined biplane LVEF threshold. In some embodiments, the first daily dose of CK-274, or a pharmaceutically acceptable salt thereof is about 5 mg of CK-274, the second daily dose of CK-274, or a pharmaceutically acceptable salt thereof, is about 5 mg or about 10 mg of CK-274, the third daily dose of CK-274, or a pharmaceutically acceptable salt thereof, is about 5 mg, about 10 mg, or about 15 mg of CK-274, and the fourth daily dose of CK-274, or a pharmaceutically acceptable salt thereof, is about 5 mg, about 10 mg, about 15 mg, or about 20 mg of CK-274. In some embodiments, the method further comprises measuring the biplane LVEF for the third echocardiogram. In some embodiments, the third time period is about 2 weeks.

In some embodiments of the above methods, the first predetermined biplane LVEF threshold is 50%, and the second predetermined biplane LVEF threshold is 55%.

In some embodiments of the above methods, the first predetermined biplane LVEF threshold is 50%, and the second predetermined biplane LVEF threshold is 60%.

In some embodiments of any of the above methods, prior to administration of CK-274, or a pharmaceutically acceptable salt thereof, the patient has end-diastolic left ventricular (LV) wall thickness ≥15 mm in one or more myocardial segments. In some embodiments of any of the above methods, prior to administration of CK-274, or a pharmaceutically acceptable salt thereof, the patient has end-diastolic left ventricular (LV) wall thickness ≥13 mm in one or more wall segments and a known disease-causing gene mutation or positive family history of HCM.

In some embodiments of any of the above methods, prior to administration of CK-274, or a pharmaceutically acceptable salt thereof, the patient has resting and post-Valsalva LVOT-G <30 mmHg.

In some embodiments of any of the above methods, prior to administration of CK-274, or a pharmaceutically acceptable salt thereof, the patient has resting LVOT-G <30 mmHg. In some embodiments of any of the above methods, prior to administration of CK-274, or a pharmaceutically acceptable salt thereof, the patient has a post-Valsalva LVOT-G <50 mmHg. In some embodiments of any of the above methods, prior to administration of CK-274, or a pharmaceutically acceptable salt thereof, the patient has resting LVOT-G <30 mmHg and a post-Valsalva LVOT-G <50 mmHg.

In some embodiments of any of the above methods, prior to administration of CK-274, or a pharmaceutically acceptable salt thereof, the patient has KCCQ-CSS score of ≥30 and ≤85.

In some embodiments of any of the above methods, prior to administration of CK-274, or a pharmaceutically acceptable salt thereof, the patient has elevated NT-proBNP. In some such embodiments, of any of the above methods, prior to administration of CK-274, or a pharmaceutically acceptable salt thereof, the patient has NT-proBNP levels >300 μg/mL.

In some such embodiments, of any of the above methods, prior to administration of CK-274, or a pharmaceutically acceptable salt thereof, the patient has NT-proBNP levels ≥900 μg/mL if in atrial fibrillation or atrial flutter. In some such embodiments, of any of the above methods, prior to administration of CK-274, or a pharmaceutically acceptable salt thereof, the patient has an NT-pro BNP ≥225 μg/mL or NT-proBNP ≥675 μg/mL if in atrial fibrillation or atrial flutter.

In some embodiments of any of the above methods, prior to administration of CK-274, or a pharmaceutically acceptable salt thereof, the patient has left ventricular ejection fraction (LVEF) ≥60%.

In some embodiments of any of the above methods, the patient is not administered disopyramide during treatment with CK-274, or a pharmaceutically acceptable salt thereof.

In some embodiments of any of the above methods, the patient has not been treated with disopyramide or an antiarrhythmic drug that has negative inotropic activity within 4 weeks prior to treatment with CK-274, or a pharmaceutically acceptable salt thereof.

In some embodiments of any of the above methods, the patient is a CYP2D6 poor metabolizer.

In some embodiments of any of the above methods, the patient is fasting when administered CK-274, or a pharmaceutically acceptable salt thereof.

In some embodiments of any of the above methods, the patient is fed when administered CK-274, or a pharmaceutically acceptable salt thereof.

In some embodiments of any of the above methods, the method does not include taking a blood sample of the patient.

In some embodiments of any of the above methods, the method does not include analyzing a blood sample of the patient.

In some embodiments of any of the above methods, the patient is administered a beta-blocker during the treatment with CK-274, or a pharmaceutically acceptable salt thereof. In some embodiments of any of the above methods, the patient is administered one or more of a beta-blocker, verapamil, diltiazem, and ranolazine.

In some embodiments, the patient is obese. In some embodiments, the patient is not obese. In some embodiments, the patient has a body mass index (BMI) of 30 or greater. In some embodiments, the patient has a body mass index (BMI) of less than 30.

Also provided herein is a method of treating non-obstructive hypertrophic cardiomyopathy (nHCM) or HCM with mid-ventricular obstruction (MVO) in a patient in need thereof, comprising administering to the patient a therapeutically effective amount of CK-274, or a pharmaceutically acceptable salt thereof.

In some embodiments of any of the above methods, the method results in one or more of the following: improvement in cardiac relaxation, beneficial cardiac remodeling, reverse cardiac remodeling, beneficial cardiac structural remodeling, beneficial cardiac functional remodeling, reversal of adverse cardiac remodeling, reduction in mean left ventricular mass index (LVMI), improvement in left ventricular (LV) filling pressures, reduction in left atrial volume index (LAVI), reduction in the categorical assessment of systolic anterior motion of the mitral valve leaflet, reduction in systolic anterior motion of the mitral valve leaflet, reduction in the frequency of eccentric mitral regurgitation, reduction in mitral regurgitation, reduction in lateral E/e′, reduction in lateral E/E, reduction in brain natriuretic peptide (BNP) and reduction in N-terminal prohormone of brain natriuretic peptide (NT-proBNP).

In some embodiments, the one or more results of the treatment occur within 1, 2, 3, 4, 5, 6, 7, 8, 9, or 10 weeks of initiating treatment with CK-274, or a pharmaceutically acceptable salt thereof.

In some embodiments of any of the above methods, a left ventricle mass index (LVMI) for the patient is reduced.

In some embodiments of any of the above methods, a left arterial volume index (LAVI) for the patient is reduced.

In some embodiments of any of the above methods, an e′ for the patient is reduced.

In some embodiments of any of the above methods, a lateral E/e′ for the patient is reduced.

In some embodiments of any of the above methods, a likelihood systolic anterior motion of the mitral valve leaflet is reduced.

In some embodiments of any of the above methods, a level of brain natriuretic peptide or N-terminal prohormone of brain natriuretic peptide (NT-proBNP) in the patient is decreased.

In some embodiments of any of the above methods, a level of cardiac troponin I in the patient is decreased.

In some embodiments of any of the above methods, left ventricular wall stress in the patient is decreased.

In some embodiments of any of the above methods, myocardial injury in the patient is decreased.

In some embodiments of any of the above methods, heart failure symptoms in the patient are reduced, for instance, the methods result in the reduction in the patient's NYHA classification.

In some embodiments of any of the above methods, the degree of mid-ventricular obstruction is reduced.

In some embodiments of any of the above methods, the method results in sustained effect(s) for at least 10 weeks, 12 weeks, 6 months, 1 year, 2 years, 3 years, 4 years, or 5 years.

In some embodiments of any of the above methods, administration for the second time period, third time period, or fourth time period may be, for example, administration for about 2 weeks, about 10 weeks, about 12 weeks, about 6 months, about 1 year, about 2 years, about 3 years, about 4 years, or about 5 years, or indefinite. As used herein, administration for an indefinite time period may indicate: administration until the patient is no longer in need of treatment; administration until there is no further effect of treatment; or administration until there is no further reason for treatment.

In some embodiments, the methods result in a reduction in the patient's NYHA classification by one or more classes, and/or a decrease in NT-proBNP, and/or a decrease in the level of troponin I (e.g., cardiac troponin I).

In some embodiments of any of the above methods, the CK-274 or pharmaceutically acceptable salt thereof is administered orally. In some embodiments, the CK-274 or pharmaceutically acceptable salt thereof is administered as a tablet. In some embodiments, the tablet comprises one or more carriers or excipients selected from the group consisting of mannitol, lactose, starch, magnesium stearate, sodium saccharine, talcum, cellulose, sodium croscarmellose, glucose, gelatin, sucrose, and magnesium carbonate. In some embodiments, the tablet comprises: (i) about 1% by weight to about 50% by weight of the CK-274 or pharmaceutically acceptable salt thereof; (ii-1) about 10% by weight to about 60% by weight of mannitol; (ii-2) about 5% by weight to about 45% by weight of microcrystalline cellulose; (iii) about 0.1% by weight to about 10% by weight of hydroxypropyl cellulose; (iv) about 1% by weight to about 10% by weight of croscarmellose sodium; (v) about 0.1% by weight to about 10% by weight of sodium lauryl sulfate; and vi) about 0.1% by weight to about 10% by weight of magnesium stearate, wherein the % by weight excludes the weight of a coating, if present. In some embodiments, the CK-274 or pharmaceutically acceptable salt thereof comprises polymorphic one or more of Form I, Form II, Form III, Form IV, Form V, and Form VI of CK-274.

### BRIEF DESCRIPTION OF THE DRAWINGS

FIG. 1 illustrates and exemplary method for treating non-obstructive hypertrophic cardiomyopathy (nHCM) or HCM with mid-ventricular obstruction (MVO) in a patient that includes titrating the daily dose of CK-274, or a pharmaceutically acceptable salt thereof.

FIG. 2 shows a schematic overview of a phase 1 clinical study for CK-274 (also referred to as aficamten). The study included SAD cohorts, MAD cohorts, a CYP2D6-PM cohort, and a food-effect cohort. The MAD and CYP2D6-PM cohorts began when a tolerated, pharmacologically active dose (reduction in LVEF of approximately 5%) was identified in the SAD cohorts. The food-effect cohort began following completion of the last SAD cohort. Criteria to stop dose escalation were met in the SAD 75-mg dose cohort, and remaining patients in this cohort received 50 mg. Subsequently, the final SAD cohort was completed using 40 mg of aficamten. CYP2D6-PM=cytochrome P450 2D6 poor metabolizer phenotype; d=day; LVEF=left ventricular ejection fraction; MAD=multiple-ascending dose; qd=once daily; SAD=single-ascending dose.

FIG. 3A shows mean (SE) maximum plasma concentration (Cₘₐₓ) of aficamten increased in a dose-proportional manner following single oral doses between 1 mg and 50 mg. FIG. 3B shows exposure (AUC₂₄) (B) of aficamten increased in a dose-proportional manner following single oral doses between 1 mg and 50 mg. AUC₂₄=area under the plasma drug concentration-time curve from 0 to 24 h; Cₘₐₓ=maximum plasma concentration; SE=standard error.

FIG. 4 shows plasma concentration over time with multiple doses of aficamten according to an exemplary clinical trial. Mean (SE) aficamten plasma concentrations are displayed. Data points are offset for clarity. Aficamten plasma concentrations increased between the 5-mg dose and the 2 higher doses; however, by day 2 there was no difference between mean concentrations of the 7.5-mg and 10-mg doses. Clearance was similar for the 5-mg and 10-mg doses, and the accumulation ratio was similar for all 3 doses. For days 7, 8, 10, 11, 12, and 13, only trough measurements are shown. For the 5-mg and 10-mg cohorts, the dosing period was 14 days with a 3-day follow-up. For the 7.5-mg cohort, dosing was extended to 17 days, with a 3-day follow-up, and confirmed that steady state was achieved after 10 to 12 days. SE=standard error.

FIG. 5A shows SAD cohorts and FIG. 5B shows MAD cohorts for an exemplary aficamten clinical trial. Mean (SE) change from baseline in LVEF is displayed. Data points are offset for clarity. In both the SAD and MAD cohorts, reductions in LVEF within the target range (5% to 15% reduction) were observed. In the SAD cohorts, there were generally small decreases in LVEF, with mean maximum reduction of 5.8% in the 50-mg group (at 1.5 h post dose). In the MAD cohort, the greatest mean reduction in LVEF from baseline occurred in the 10-mg group (mean change of 5.0% 1.5 h after dosing on day 14). LVEF=left ventricular ejection fraction; MAD=multiple-ascending dose; qd, once daily; SAD=single-ascending dose; SE=standard error.

FIG. 6A shows analysis of the SAD cohorts according to an exemplary clinical trial, and shows that, as the plasma concentration of aficamten increased, there was a trend toward a decrease in LVEF. FIG. 6B shows analysis of the MAD cohorts from an exemplary clinical trial, and shows minimal suppression of LVEF in most participants at plasma aficamten concentrations of ≤180 ng/ml. CI=confidence interval; LVEF=left ventricular ejection fraction; MAD=multiple-ascending dose; SAD=single-ascending dose.

FIG. 7 shows the design of an exemplary clinical trial for CK-274 (aficamten).

FIG. 8 shows the design of an open label extension of an exemplary clinical trial for CK-274 (aficamten).

FIG. 9 shows mean left ventricular ejection fraction for patients during the titration period of an exemplary clinical trial of aficamten (Example 2, interim results).

FIG. 10 shows the dose level achieved for patients by the end of the titration period of an exemplary clinical trial of aficamten (Example 2, interim results).

FIG. 11 shows mean NT-proBNP levels in patients over time during an exemplary clinical trial of aficamten (Example 2, interim results).

FIG. 12 shows mean hs-cTnI levels in patients over time during an exemplary clinical trial of aficamten (Example 2, interim results).

FIG. 13 shows proportion of patients having each NYHA functional classification over time during an exemplary clinical trial of aficamten (Example 2, interim results).

FIG. 14 shows median KCCQ-CSS and SAQ-AF for patients over time during an exemplary clinical trial of aficamten (Example 2, interim results).

FIG. 15 shows proportion of patients having categorical changes in KCQ-CSS at week 10 of an exemplary clinical trial of aficamten (Example 2, interim results).

FIG. 16 shows the design of an exemplary clinical trial of aficamten (Example 4).

FIG. 17 shows left ventricular ejection fraction for patients during the treatment period of an exemplary clinical trial of aficamten (Example 2, second interim results).

FIG. 18 shows mean KCCQ-CSS for patients over time during an exemplary clinical trial of aficamten (Example 2, second interim results).

FIG. 19 shows proportion of patients having categorical changes in KCQ-CSS at week 10 of an exemplary clinical trial of aficamten (Example 2, second interim results).

FIG. 20 shows proportion of patients having each NYHA functional classification over time during an exemplary clinical trial of aficamten (Example 2, second interim results).

FIG. 21 shows mean SAQ-AF for patients over time during an exemplary clinical trial of aficamten (Example 2, second interim results).

FIG. 22 shows proportional change from baseline of NT-proBNP levels and hs-cTnI levels in patients over time during an exemplary clinical trial of aficamten (Example 2, second interim results).

FIG. 23A shows a responder analysis for patients with BMI <30 or ≥30 in an exemplary clinical trial of aficamten (Example 2, second interim results).

FIG. 23B shows a responder analysis for patients being treated with beta-blockers or without beta-blockers in an exemplary clinical trial of aficamten (Example 2, second interim results).

FIG. 23C shows a responder analysis for patients with elevated TnI or E/e′>13 or non-elevated TnI or E/e′ in an exemplary clinical trial of aficamten (Example 2, second interim results).

FIG. 23D shows a responder analysis for patients with a genetic or family history of HCM or without a genetic or family history of HCM in an exemplary clinical trial of aficamten (Example 2, second interim results).

FIG. 24 shows reduction in NT-proBNP and hs-Troponin I levels, as well as symptom improvement, in patients with mid-ventricular obstruction in an exemplary clinical trial of aficamten (Example 2, second interim results).

FIG. 25 shows change in Doppler measures of diastolic function for patients in an exemplary clinical trial of aficamten (Example 2, second interim results).

FIG. 26A shows an experimental X-ray powder diffraction (XRPD) pattern of polymorphic Form I of CK-274.

FIG. 26B shows differential scanning calorimetry (DSC) and thermogravimetric analysis (TGA) graphs of polymorphic Form I of CK-274.

FIG. 26C shows a Dynamic Vapor Sorption (DVS) graph of polymorphic Form I of CK-274.

FIG. 27A shows an experimental XRPD pattern of polymorphic Form II of CK-274.

FIG. 27B shows DSC and TGA graphs of polymorphic Form II of CK-274.

FIG. 28A shows an experimental XRPD pattern of a mixture of polymorphic Forms I and III of CK-274.

FIG. 28B shows DSC and TGA graphs of a mixture of polymorphic Forms I and III of CK-274.

FIG. 29A shows an experimental XRPD pattern of polymorphic Form IV of CK-274.

FIG. 29B shows DSC and TGA graphs of polymorphic Form IV of CK-274.

FIG. 30 shows an experimental XRPD pattern and two simulated patterns of polymorphic Form V of CK-274 (from top to bottom: simulated at 223K; simulated at 273K; experimental).

FIG. 31A shows two experimental XRPD patterns of polymorphic Form VI of CK-274: (a) at top, the XRPD of Form VI taken before drying; and (b) at bottom, after drying (oven, vacuum, 24 hours, at 25° C.).

FIGS. 31B and 31C show TGA graphs of polymorphic Form VI of CK-274. FIG. 31B shows the weight loss of an oven-dried sample (oven, vacuum, overnight, at 25° C.) of Form VI over the range of 25-300° C. FIG. 31C shows the TGA plot over the range of 25-300° C. for a sample of Form VI that was subjected to oven-drying (oven, vacuum, overnight, at 25° C.) and further heating at 150° C. prior to thermogravimetric analysis.

FIGS. 31D and 31E show DSC graphs of polymorphic Form VI of CK-274. FIG. 31D shows the DSC plot of an oven-dried sample (oven, vacuum, overnight, at 25° C.) of Form VI over the range of 25-300° C. FIG. 31E shows the DSC plot over the range of 25-300° C. for a sample of Form VI that was subjected to oven-drying (oven, vacuum, overnight, at 25° C.) and further heating at 150° C. prior to thermogravimetric analysis.

### DETAILED DESCRIPTION

Described herein is a cardiac myosin inhibitor (CK-3773274, also referred to as CK-274 or aficamten) and various methods, e.g., for treating non-obstructive hypertrophic cardiomyopathy or hypertrophic cardiomyopathy with mid-ventricular obstruction, using the cardiac myosin inhibitor. Treatment methods may include adjusting a dose, for example to increase, decrease or maintain a dose, based on the results of one or more biplane left ventricular ejection fraction (LVEF) measurements. These measurements may be taken, for example, using an echocardiogram.

CK-3773274 (CK-274) is a small molecule cardiac myosin inhibitor having the structure shown below.

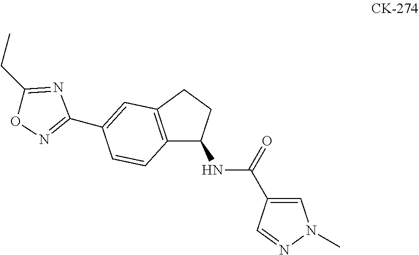 [SMILES: CCc1nc(-c2ccc3c(c2)CC[C@H]3NC(=O)c2cnn(C)c2)no1]

The chemical name of CK-274 is (R)—N-(5-(5-ethyl-1,2,4-oxadiazol-3-yl)-2,3-dihydro-1H-inden-1-yl)-1-methyl-1H-pyrazole-4-carboxamide. The small molecule inhibitor may be, for example, orally administered to a patient for the treatment of non-obstructive hypertrophic cardiomyopathy (nHCM) or HCM with MVO.

CK-274 has been described in WO 2019/144041, which is incorporated by reference herein. CK-274, or a pharmaceutically acceptable salt thereof, may be obtained following the methods described therein. CK-274 used in the disclosed methods can be present as a pharmaceutically acceptable salt, solvate, hydrate, polymorph, or combination thereof, and can be formulated into any suitable pharmaceutical formulation. CK-274 can also be present in its free base form. Polymorphs of CK-274 have been described in WO 2021/011807, which is incorporated by reference herein. Formulations of CK-274 have been described in WO 2021/011808, which is incorporated by reference herein. CK-274 was designed to reduce the hypercontractility that is associated with hypertrophic cardiomyopathy (HCM). Without being bound by theory, in preclinical models, CK-274 reduces myocardial contractility by binding directly to cardiac myosin at a distinct and selective allosteric binding site, thereby preventing myosin from entering a force producing state. CK-274 reduces the number of active actin-myosin cross bridges during each cardiac cycle and consequently reduces myocardial contractility. This mechanism of action may be therapeutically effective in conditions characterized by excessive hypercontractility, such as HCM (e.g., non-obstructive HCM, also referred to as nHCM; or HCM with MVO).

### Definitions

As used in the present specification, the following words and phrases are generally intended to have the meanings as set forth below, except to the extent that the context in which they are used indicates otherwise.

Throughout this application, unless the context indicates otherwise, references to CK-3773274, CK-274, or aficamten include amorphous forms thereof or polymorphs thereof, including any one of polymorphic Forms I, II, III, IV, V, or VI as described herein, or a mixture thereof.

Reference to “about” a value or parameter herein includes (and describes) that value or parameter per se, and any value or parameter 5% above or 5% below said parameter. For example, description to “about X” includes description of “X” and “X+/−5%”.

“NYHA classification” or “NYHA class” refers to the New York Heart Association functional classification of heart failure symptoms. Descriptions of each of NYHA classes I, II, III, and IV can be found in “Classes of Heart Failure”, American Heart Association, https://www.heart.org/en/health-topics/heart-failure/what-is-heart-failure/classes-of-heart-failure, adapted from: 1) Dolgin M, Association N Y H, Fox A C, Gorlin R, Levin R I, New York Heart Association. Criteria Committee. “Nomenclature and criteria for diagnosis of diseases of the heart and great vessels”. 9th ed. Boston, MA: Lippincott Williams and Wilkins; Mar. 1, 1994; and 2) Criteria Committee, New York Heart Association, Inc. Diseases of the Heart and Blood Vessels. Nomenclature and Criteria for diagnosis, 6th edition Boston, Little, Brown and Co. 1964, p 114. Briefly, NYHA class I indicates that the patient has no limitation of physical activity; ordinary physical activity does not cause undue fatigue, palpitation, dyspnea (shortness of breath). NYHA class II indicates that the patient has slight limitation of physical activity; comfortable at rest; ordinary physical activity results in fatigue, palpitation, dyspnea (shortness of breath). NYHA class III indicates that the patient has marked limitation of physical activity; comfortable at rest; less than ordinary physical activity causes fatigue, palpitation, or dyspnea. NYHA class IV indicates that the patient is unable to carry on any physical activity without discomfort; symptoms of heart failure at rest; if any physical activity is undertaken, discomfort increases.

The term “pharmaceutically acceptable salt” refers to a salt of any of the compounds herein that are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

If the compound described herein is obtained as an acid addition salt, the free base can be obtained by basifying a solution of the acid salt. Conversely, if the compound is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds (see, e.g., Berge et al., Pharmaceutical Salts, J. Pharmaceutical Sciences, January 1977, 66(1), 1-19). Those skilled in the art will recognize various synthetic methodologies that may be used to prepare pharmaceutically acceptable addition salts.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The terms “patient,” “individual,” and “subject” refer to an animal, such as a mammal. Mammals include, for example, mice, rats, dogs, cats, pigs, sheep, horses, cows and humans. In some embodiments, the patient or subject is a human, for example a human that has been or will be the object of treatment, observation or experiment. The compounds, compositions and methods described herein can be useful in both human therapy and veterinary applications.

The term “therapeutically effective amount” or “effective amount” refers to that amount of a compound disclosed and/or described herein that is sufficient to affect treatment, as defined herein, when administered to a patient in need of such treatment. A therapeutically effective amount of a compound may be an amount sufficient to treat a disease responsive to modulation of the cardiac sarcomere. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

“Treatment” (and related terms, such as “treat”, “treated”, “treating”) includes one or more of: inhibiting a disease or disorder; slowing or arresting the development of clinical symptoms of a disease or disorder; and/or relieving a disease or disorder (i.e., causing relief from or regression of clinical symptoms). The term covers both complete and partial reduction or prevention of the condition or disorder, and complete or partial reduction of clinical symptoms of a disease or disorder. Thus, compounds described and/or disclosed herein may prevent an existing disease or disorder from worsening, assist in the management of the disease or disorder, or reduce or eliminate the disease or disorder.

Reference to any dose amount of a compound or pharmaceutically acceptable salt thereof described herein (e.g., 5 mg, 10 mg, 20 mg, etc. of CK-274) refers to the amount (i.e., equivalent mass) of said compound without any salt.

### Treatment of Non-Obstructive Hypertrophic Cardiomyopathy

As further described herein a therapeutically effective amount of CK-274 may be administered to a patient for the treatment of non-obstructive hypertrophic cardiomyopathy. CK-274 may be administered at a constant dose level. CK-274 may be administered at a titrated dose level. For example, the dose of CK-274 may be adjusted depending on the patient's response to the drug. That is, the dose of the CK-274 may be periodically increased, decreased, maintained, or suspended depending on the measurement of a drug response, such as one or more of a left ventricular biplane left ventricular ejection fraction (LVEF) measurements.

CK-274 is administered at a therapeutically effective dosage, e.g., a dosage sufficient to provide treatment for the disease state. For a human, the daily dose may be between about 1 mg and about 50 mg. For example, the daily dose may be about 5 mg, about 10 mg, about 15 mg, about 20 mg, or about 30 mg, or any amount therebetween. A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

In one example, a patient is treated for non-obstructive hypertrophic cardiomyopathy by administering to the patient a daily dose of about 5 mg to about 15 mg of CK-274. In one example, a patient is treated for non-obstructive hypertrophic cardiomyopathy by administering to the patient a daily dose of about 5 mg CK-274. In one example, a patient is treated for non-obstructive hypertrophic cardiomyopathy by administering to the patient a daily dose of about 10 mg CK-274. In one example, a patient is treated for non-obstructive hypertrophic cardiomyopathy by administering to the patient a daily dose of about 15 mg CK-274. In one example, a patient is treated for non-obstructive hypertrophic cardiomyopathy by administering to the patient a daily dose of about 20 mg CK-274.

In some embodiments, provided are methods for the treatment for non-obstructive hypertrophic cardiomyopathy comprising administration of CK-274, or a pharmaceutically acceptable salt thereof, or a pharmaceutical composition containing CK-274, or a pharmaceutically acceptable salt thereof. In some embodiments, the non-obstructive hypertrophic cardiomyopathy is treatment refractory non-obstructive hypertrophic cardiomyopathy. In some embodiments, provided are methods for the treatment for non-obstructive hypertrophic cardiomyopathy, treatment refractory hypertrophic cardiomyopathy, or treatment refractive non-obstructive hypertrophic cardiomyopathy, comprising administration of CK-274, or a pharmaceutically acceptable salt thereof, or a pharmaceutical composition containing CK-274, or a pharmaceutically acceptable salt thereof.

During the course of treatment for non-obstructive hypertrophic cardiomyopathy, the dose of CK-274 administered to the patient may be titrated, for example by increasing, decreasing, maintaining, or suspending the dose. Titration may occur once during treatment, or may be performed iteratively separated by a period of time. For example, in some implementations, the dose of CK-274 is titrated two or more times (e.g., 3, 4, 5 or more) during the course of treatment. In some embodiments, a new daily dose amount is administered to the patient at a constant amount for about 1 week to about 8 weeks (or about 2 weeks to about 6 weeks, or about 4 weeks) before the daily dose amount is titrated. In some embodiments, a new daily dose amount is administered to the patient at a constant amount for about 2 weeks before being titrated. For example, a first daily dose may be administered to the patient for about 2 weeks before a first titration, wherein the daily dose amount is increased, decreased or maintained. The second titration may then occur approximately 2 weeks after the first titration. Titration of the dose allows the dose to be personalized to the patient's response to the drug, thus maximizing the potential treatment effect for patients.

Titration of the dose can be based on biplane left ventricular ejection fraction (LVEF measured in the patient. The measurement or measurements may be determined, for example, using an echocardiogram. The echocardiogram may be taken following administration of the daily dose, for example about 1 hour to about 3 hours following administration of the dose. In some embodiments, the echocardiogram is taken about 2 hours following administration of the daily dose.

In some embodiments, an initial daily dose of about 5 mg, about 10 mg, about 15 mg, about 20 mg, or about 30 mg CK-274, or any amount therebetween, is administered to the patient. After a period of time (e.g., about 2 weeks), biplane LVEF is measured, for example by echocardiography, following the administration of the dose (for example, about 1-3 hours, or about 2 hours following the administration of the dose). If the biplane LVEF is at or above a predetermined biplane LVEF threshold (e.g., about 40% or more, about 45% or more, about 50% or more, about 55% or more, or about 60% or more), the daily dose is increased. In some embodiments, the daily dose is increased if the biplane LVEF is at or above a predetermined biplane LVEF threshold of about 55%. In some embodiments, the daily dose is increased if the biplane LVEF is at or above a predetermined biplane LVEF threshold of about 60%. The dose may be decreased or terminated if the biplane LVEF is below the biplane LVEF threshold. In some embodiments, the biplane LVEF threshold is about 50%.

After a period of time (e.g., about 2 weeks) of the patient being administered the first titrated dose, the dose may be again titrated (i.e., increased, decreased, or maintained) or suspended based on the biplane LVEF of the patient, for example using the same threshold parameters as discussed above.

Provided herein is a method of treating non-obstructive hypertrophic cardiomyopathy (nHCM) in a patient eligible for septal reduction therapy (SRT), which comprises administering to the patient a therapeutically effective amount of CK-274, or a pharmaceutically acceptable salt thereof. Also provided herein is a method of treating nHCM in a patient in need of SRT, which comprises administering to the patient a therapeutically effective amount of CK-274, or a pharmaceutically acceptable salt thereof, wherein the method precludes the need for SRT in the patient. In some embodiments, the SRT is myectomy. In some embodiments, the SRT is alcohol septal ablation.

Further provided herein is a method of treating non-obstructive hypertrophic cardiomyopathy (nHCM) in a patient with heart failure symptoms, comprising administering to the patient a therapeutically effective amount of CK-274, or a pharmaceutically acceptable salt thereof, wherein the method results in a reduction of heart failure symptoms as assessed by NYHA classification. In some embodiments of the foregoing, the methods improve heart failure symptoms by at least one NYHA class in the patient, for instance, by one or two NYHA class(es). In some embodiments of the foregoing, the methods convert patients from NYHA class III to class II or class I. In some embodiments of the foregoing, the methods convert patients from NYHA class III to class II. In some embodiments of the foregoing, the methods convert patients from NYHA class III to class I. In some embodiments of the foregoing, the methods convert patients from NYHA class II to class I. In some embodiments of the foregoing, reduction of heart failure symptoms occurs within ten weeks of initiating treatment with CK-274, or a pharmaceutically acceptable salt thereof.

In some embodiments, titration of the daily dose of CK-274, or a pharmaceutically acceptable salt thereof, is based on the results of an echocardiogram that includes a biplane LVEF. For example, based on the results of the biplane LVEF, the daily dose of CK-274, or a pharmaceutically acceptable salt thereof, may be increased, maintained, or decreased (or terminated, for example if the subject is already receiving the lowest (e.g., first) daily dose). A first daily dose of CK-274, or a pharmaceutically acceptable salt thereof, is administered to the patient for a first time period (e.g., about two weeks). A second daily dose for the subject, or termination of administration of CK-274 or the pharmaceutically acceptable salt thereof, is then selected based on a biplane LVEF for the patient acquired after the first time period. The administration of CK-274 or the pharmaceutically acceptable salt thereof may be terminated if the biplane LVEF of the echocardiogram is below a first predetermined biplane LVEF threshold (e.g., 50%) and the patient is already receiving the lowest (e.g., first) daily dose. If the biplane LVEF of the echocardiogram is below a first predetermined biplane LVEF threshold (e.g., 50%) and the patient is not already receiving the lowest daily dose, then the daily dose may be decreased (i.e., the second daily dose is lesser than the first daily dose). If the biplane LVEF is at or above the first predetermined biplane threshold and below a second predetermined biplane LVEF threshold (e.g., 55%), then the daily dose is maintained (i.e., the second daily dose is the same as the first daily dose). If the biplane LVEF is at or above the second predetermined biplane threshold, the daily dose may be increased (i.e., the second daily dose is greater than the first daily dose). In some embodiments, the second predetermined biplane LVEF threshold is 60%.

The second daily dose may be administered to the patient for a second time period (e.g., about two weeks) before again being titrated based on the results of a second echocardiogram that includes a biplane LVEF for the patient acquired after the second time period. (The second time period may be, for example, about 2 weeks, about 10 weeks, about 12 weeks, about 6 months, about 1 year, about 2 years, about 3 years, about 4 years, or about 5 years, or indefinite.) For example, a third daily dose, or termination of the administration, may be selected or the administration terminated based on the second echocardiogram and the second daily dose. If the second daily dose is the same as (or lower than) the first daily dose (e.g., if the second daily dose is the lowest dose) and the biplane LVEF of the second echocardiogram is below the first predetermined biplane LVEF threshold, the administration may be terminated. If the second daily dose is higher than the first daily dose and the biplane LVEF of the second echocardiogram is below the first predetermined biplane LVEF threshold, the third daily dose may be decreased relative to the second daily dose, for example to the amount of the first daily dose. If the biplane LVEF is at or above the first predetermined biplane LVEF threshold and below the second predetermined biplane LVEF threshold, the daily dose may be maintained (i.e., the third daily dose is the same as the second daily dose). The third daily dose may be increased relative to the second daily dose if the biplane LVEF of the second echocardiogram is above the second predetermined biplane LVEF threshold. The third daily dose is then administered to the patient for a third time period (e.g., two weeks). The third time period may be, for example, about 2 weeks, about 10 weeks, about 12 weeks, about 6 months, about 1 year, about 2 years, about 3 years, about 4 years, or about 5 years, or indefinite.

Titration of the daily dose may be repeated for additional rounds, if desired, to select a fourth daily dose of CK-274, or a pharmaceutically acceptable salt thereof or termination of administration. For example, a third echocardiogram comprising a biplane LVEF may be acquired for the patient after the third time period, and a fourth daily dose of CK-274, or a pharmaceutically acceptable salt thereof, may be selected based on the third echocardiogram and the third daily dose. If the biplane LVEF of the third echocardiogram is below the first predetermined biplane LVEF threshold and the third daily dose is the same as (or lower than) the first daily dose, the administration of CK-274, or a pharmaceutically acceptable salt thereof, may be terminated. If the third daily dose is higher than the first daily dose, and the biplane LVEF of the third echocardiogram is below the first predetermined biplane LVEF threshold, then the fourth daily dose is decreased relative to the third daily dose. If the biplane LVEF of the third echocardiogram is below the first predetermined biplane LVEF threshold and the third daily dose is the same as (or lower than) the first daily dose (e.g., if the third daily dose is the lowest dose), the administration of CK-274, or a pharmaceutically acceptable salt thereof, may be terminated. If the third daily dose is higher than the lowest (e.g., first) daily dose, and the biplane LVEF of the third echocardiogram is below the first predetermined biplane LVEF threshold, then the fourth daily dose is decreased relative to the third daily dose. If the biplane LVEF of the third echocardiogram is at or above the predetermined biplane LVEF threshold, then the fourth daily dose may be the same as the third daily dose. If the biplane LVEF is at or above the first predetermined biplane LVEF threshold and below the second predetermined biplane LVEF threshold, the daily dose may be maintained (i.e., the fourth daily dose is the same as the third daily dose). The fourth daily dose may be increased relative to the third daily dose if the biplane LVEF of the second echocardiogram is above the second predetermined biplane LVEF threshold. The fourth daily dose is then administered to the patient for a fourth time period (e.g., two weeks). The fourth time period may be, for example, about 2 weeks, about 10 weeks, about 12 weeks, about 6 months, about 1 year, about 2 years, about 3 years, about 4 years, or about 5 years, or indefinite.

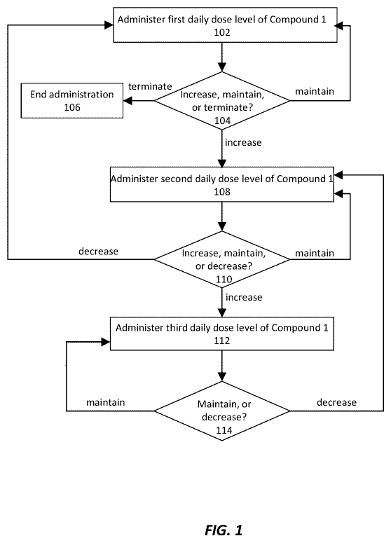

FIG. 1 illustrates and exemplary method for treating non-obstructive hypertrophic cardiomyopathy (nHCM) in a patient that includes titrating the daily dose of CK-274, or a pharmaceutically acceptable salt thereof. The exemplary method shown in FIG. 1 provides three daily dose levels, with a first daily dose level being the lowest daily dose level, but may be readily modified to include additional or fewer dose levels, or may be further modified such that the first daily dose level is not the lowest daily dose level. At 102, the first daily dose level (e.g., about 5 mg) of CK-274, or a pharmaceutically acceptable salt thereof, is administered to a patient. After a first time period, the daily dose level is increased or maintained, or the administration is terminated, at 104. The selection may be based on a first echocardiogram acquired for the patient after the first time period. Termination of administration 106 may be selected if the biplane LVEF of the first echocardiogram is below a predetermined biplane LVEF threshold (e.g., 50%), wherein the no further dose of CK-274 or pharmaceutically acceptable salt thereof is administered to the patient. Maintenance of the first daily dose level (e.g., about 5 mg) may be selected when the biplane LVEF is at or above the predetermined biplane LVEF threshold (e.g., 50%) and below a second predetermined biplane LVEF threshold (e.g., 55%). If maintenance is selected, the first daily dose level of CK-274, or a pharmaceutically acceptable salt thereof, is administered to the patient at 102 for a second time period, and, optionally, the daily dose may be re-titrated at 104 after the second time period. The daily dose level may be increased to a second daily dose level (e.g., 10 mg) when the biplane LVEF is at or above the second predetermined biplane LVEF threshold (e.g., 55%). If an increase in the daily dose level is selected, the second daily dose level of CK-274, or a pharmaceutically acceptable salt thereof, is administered to the patient for the second time period at 108.

If the second daily dose level of CK-274, or a pharmaceutically acceptable salt thereof, (e.g., 10 mg) is administered to the patient at 108, the daily dose may be re-titrated (i.e., select an increase, decrease, or maintenance of the daily dose) based on an echocardiogram at 110. The daily dose may be decreased to the first daily dose level (e.g., from 10 mg to 5 mg) if the biplane LVEF of the echocardiogram is below a predetermined biplane LVEF threshold (e.g., 50%). If the daily dose is decreased to the first daily dose level, the first daily dose level is administered to the patient at 102. Maintenance of the second daily dose level (e.g., about 10 mg) may be selected when the biplane LVEF is at or above the predetermined biplane LVEF threshold (e.g., 50%) and below a second predetermined biplane LVEF threshold (e.g., 55%). If maintenance is selected, the second daily dose level of CK-274, or a pharmaceutically acceptable salt thereof, is administered to the patient at 108 for a further time period, and, optionally, the daily dose may be re-titrated at 110 after said time period. The daily dose level may be increased to a third daily dose level (e.g., 15 mg) when the biplane LVEF is at or above the second predetermined biplane LVEF threshold (e.g., 55%). If an increase in the daily dose level is selected, the second daily dose level of CK-274, or a pharmaceutically acceptable salt thereof, is administered to the patient for the time period at 112.

If the third daily dose level of CK-274, or a pharmaceutically acceptable salt thereof, (e.g., 10 mg) is administered to the patient at 112, the daily dose may be re-titrated (i.e., select a decrease or maintenance of the daily dose) based on an echocardiogram at 114. The daily dose may be decreased to the second daily dose level (e.g., from 15 mg to 10 mg) if the biplane LVEF of the echocardiogram is below a predetermined biplane LVEF threshold (e.g., 50%). If the daily dose is decreased to the second daily dose level, the second daily dose level is administered to the patient at 108. Maintenance of the third daily dose level (e.g., about 15 mg) may be selected when the biplane LVEF is at or above the predetermined biplane LVEF threshold (e.g., 50%). If maintenance is selected, the third daily dose level of CK-274, or a pharmaceutically acceptable salt thereof, is administered to the patient at 112 for a further time period, and, optionally, the daily dose may be re-titrated at 114 after said time period.

In the exemplary method shown in FIG. 1, the first daily dose level is the minimum dose, and therefore may not be further decreased. Nevertheless, in other embodiments, the first daily dose level may be other than the minimum dose, and therefore may be decreased to a lower dose level (e.g., from 10 mg to 5 mg) if the biplane LVEF of the echocardiogram is below a predetermined biplane LVEF threshold (e.g., 50%).

In the exemplary method shown in FIG. 1, the third daily dose level is the maximum dose, and therefore may not be further increased. Nevertheless, in other embodiments, additional dose levels may be available and the daily dose may be further increased at 114. The method shown in FIG. 1, at 114 a selection is made to maintain the third daily dose level or decrease the daily dose level based on an echocardiogram. The daily dose may be decreased to the second daily dose level (e.g., from 15 mg to 10 mg) if the biplane LVEF of the echocardiogram is below a predetermined biplane LVEF threshold (e.g., 50%). If the daily dose is decreased to the second daily dose level, the second daily dose level is administered to the patient at 108. Maintenance of the third daily dose level (e.g., about 15 mg) may be selected when the biplane LVEF is at or above the predetermined biplane LVEF threshold (e.g., 50%). If maintenance is selected, the third daily dose level of CK-274, or a pharmaceutically acceptable salt thereof, is administered to the patient at 112 for a further time period, and, optionally, the daily dose may be re-titrated at 114 after said time period. In embodiments wherein additional dose levels may be available and the daily dose may be further increased at 114, the daily dose may be increased to a fourth daily dose level (e.g., about 20 mg) if the biplane LVEF is at or above the second predetermined biplane LVEF threshold (e.g., 60%).

Exemplary daily dose increases include an increase from about 5 mg to about 10 mg CK-274, or about 10 mg to about 15 mg CK-274. Other exemplary daily dose increases include an increase from about 5 mg to about 10 mg CK-274, from about 10 mg to about 15 mg CK-274, or from about 15 mg to about 20 mg CK-274. Other dose increases may be readily envisioned, for instance, increasing a given initial daily dose by about 1 mg, about 2 mg, about 3 mg, about 4 mg, about 5 mg, or about 10 mg, or any amount therebetween. Exemplary daily dose decreases include a decrease from about 15 mg to about 10 mg, or about 10 mg to about 5 mg. Other exemplary daily dose decreases include a decrease from about 20 mg to about 15 mg, from about 15 mg to about 10 mg, or from about 10 mg to about 5 mg. Other dose decreases may be readily envisioned, for instance, decreasing a given initial daily dose by about 1 mg, about 2 mg, about 3 mg, about 4 mg, about 5 mg, or about 10 mg, or any amount therebetween.

Exemplary embodiments of the methods described herein comprise administering a first daily dose or Dose 1 (e.g., a first daily dose of between about 1 mg and about 20 mg, such as 1 mg, 2 mg, 3 mg, 4 mg, 5 mg, 6 mg, 7 mg, 8 mg, 9 mg, 10 mg, 15 mg, or 20 mg) or any amount therebetween) of CK-274, or a pharmaceutically acceptable salt thereof, for a first period (e.g., about one week, two weeks, three weeks, four weeks, five weeks, six weeks, seven weeks, eight weeks, nine weeks, ten weeks, twelve weeks, or any length of time therebetween), followed by maintaining the daily dose, decreasing the daily dose (e.g., decreasing the daily dose by between about 1 mg and about 10 mg, such as decreasing the daily dose by 1 mg, 2 mg, 3 mg, 4 mg, 5 mg 6 mg, 7 mg, 8 mg, 9 mg, or 10 mg, or any amount therebetween), increasing the daily dose (e.g., increasing the daily dose by between about 1 mg and about 10 mg, such as increasing the daily dose by 1 mg, 2 mg, 3 mg, 4 mg, 5 mg 6 mg, 7 mg, 8 mg, 9 mg, or 10 mg, or any amount therebetween), or terminating administration based on the biplane LVEF of the patient to arrive at a second daily dose.

Another exemplary embodiment of the methods described herein comprises administering a first daily dose of CK-274, or a pharmaceutically acceptable salt thereof, for about two weeks, followed by maintaining the daily dose, decreasing the daily dose by about 5 mg, increasing the daily dose by about 5 mg, or terminating administration based on the biplane LVEF of the patient to arrive at a second daily dose. Another exemplary embodiment of the methods described herein comprises administering a first daily dose of CK-274, or a pharmaceutically acceptable salt thereof, for about three weeks, followed by maintaining the daily dose, decreasing the daily dose by about 5 mg, increasing the daily dose by about 5 mg, or terminating administration based on the biplane LVEF of the patient to arrive at a second daily dose. Another exemplary embodiment of the methods described herein comprises administering a first daily dose of CK-274, or a pharmaceutically acceptable salt thereof, for about two weeks, followed by maintaining the daily dose, decreasing the daily dose by about 10 mg, increasing the daily dose by about 10 mg, or terminating administration based on the biplane LVEF of the patient to arrive at a second daily dose. Another exemplary embodiment of the methods described herein comprises administering a first daily dose of CK-274, or a pharmaceutically acceptable salt thereof, for about three weeks, followed by maintaining the daily dose, decreasing the daily dose by about 10 mg, increasing the daily dose by about 10 mg, or terminating administration based on the biplane LVEF of the patient to arrive at a second daily dose. Another exemplary embodiment of the methods described herein comprises administering a first daily dose of CK-274, or a pharmaceutically acceptable salt thereof, for between about 2 and about 12 weeks, followed by maintaining the daily dose, decreasing the daily dose by about 10 mg, increasing the daily dose by about 10 mg, or terminating administration based on the biplane LVEF of the patient to arrive at a second daily dose.

Treatment for non-obstructive hypertrophic cardiomyopathy can result in improved exercise capacity and/or relieve symptoms in patients with hyperdynamic ventricular contraction resulting from non-obstructive hypertrophic cardiomyopathy. In some embodiments, the method includes administering a therapeutically effective daily dose of CK-274, or a pharmaceutically acceptable salt thereof, to an individual with non-obstructive hypertrophic cardiomyopathy, thereby improving the exercise capacity of the individual. In some embodiments, exercise capacity can be measured by one or more cardiopulmonary exercise testing (CPET) parameters, or a composite of one or more CPET parameters. In some embodiments, exercise capacity can be measured by peak oxygen uptake (pVO₂) (maximal exercise capacity) and/or minute ventilation/carbon dioxide production (VE/VCO₂) slope (sub-maximal exercise capacity). In some embodiments, the method includes administering a therapeutically effective daily dose of CK-274, or a pharmaceutically acceptable salt thereof, to an individual with non-obstructive hypertrophic cardiomyopathy, thereby resulting in a change in pVO2 of ≥1.0 mL/kg/min from baseline, and ≥1 class improvement in NYHA Functional Class in the individual.). In some embodiments, the method includes administering a therapeutically effective daily dose of CK-274, or a pharmaceutically acceptable salt thereof, to an individual with non-obstructive hypertrophic cardiomyopathy, thereby resulting in a change in pVO2 of ≥2.0 mL/kg/min from baseline, and no worsening in NYHA Functional Class. In some embodiments, the method includes administering a therapeutically effective daily dose of CK-274, or a pharmaceutically acceptable salt thereof, to an individual with non-obstructive hypertrophic cardiomyopathy, thereby relieving one or more symptoms of hyperdynamic ventricular contraction.

In some embodiments, a therapeutically effective amount of CK-274, or a pharmaceutically acceptable salt thereof, is administered to a patient with non-obstructive hypertrophic cardiomyopathy to treat the non-obstructive hypertrophic cardiomyopathy, wherein the biplane LVEF is maintained at or above 50%. In some embodiments, in response to administration of the therapeutically effective amount of CK-274, or a pharmaceutically acceptable salt thereof, the biplane LVEF decreases by less than about 20%, less than about 15%, less than about 10%, or less than about 5%. The maintenance interval of the biplane LVEF may be about 1 week or longer, about 2 weeks or longer, about 3 weeks or longer, about 4 weeks or longer, about 5 weeks or longer, about 6 weeks or longer, about 8 weeks or longer, or about 10 weeks or longer of daily dose administration.

In some embodiments, a therapeutically effective amount of CK-274, or a pharmaceutically acceptable salt thereof, is administered to a patient with non-obstructive hypertrophic cardiomyopathy, thereby decreasing a left ventricle mass index (LVMI) for the patient. In response to administration of the therapeutically effective amount of CK-274, or a pharmaceutically acceptable salt thereof, the LVMI can decrease by about 1 g/m² or more, by about 1.5 g/m² or more, by about 2 g/m² or more, by about 2.5 g/m² or more, by about 3 g/m² or more, by about 3.5 g/m² or more, or by about 4 g/m² or more. In some embodiments, in response to administration of the therapeutically effective amount of CK-274, or a pharmaceutically acceptable salt thereof, the LVMI decreases by about 1 g/m² to about 10 g/m² mmHg, such as by about 1 g/m² to about 6 g/m², or about 2 g/m² to about 5 g/m². The decrease in LVMI may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of CK-274, or a pharmaceutically acceptable salt thereof, is administered to a patient with non-obstructive hypertrophic cardiomyopathy, thereby decreasing a left arterial volume index (LAVI) for the patient. In response to administration of the therapeutically effective amount of CK-274, or a pharmaceutically acceptable salt thereof, the LAVI can decrease by about 0.5 mL/m² or more, by about 1 mL/m² or more, by about 1.5 mL/m² or more, by about 2 mL/m² or more, or by about 2.5 mL/m² or more. In some embodiments, in response to administration of the therapeutically effective amount of CK-274, or a pharmaceutically acceptable salt thereof, the LAVI decreases by about 0.5 mL/m² to about 5 mL/m² mmHg, such as by about 0.5 mL/m² to about 4 g/m², or about 1 mL/m² to about 3 mL/m². The decrease in LAVI may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of CK-274, or a pharmaceutically acceptable salt thereof, is administered to a patient with non-obstructive hypertrophic cardiomyopathy, thereby decreasing an e′ value for the patient. In response to administration of the therapeutically effective amount of CK-274, or a pharmaceutically acceptable salt thereof, the e′ value can decrease by about 0.1 cm/s or more, by about 0.15 cm/s or more, by about 0.2 cm/s or more, or by about 0.25 cm/s or more. In some embodiments, in response to administration of the therapeutically effective amount of CK-274, or a pharmaceutically acceptable salt thereof, the e′ value decreases by about 0.05 cm/s to about 0.3 cm/s, such as by about 0.1 cm/s to about 0.25 cm/s, or about 0.15 cm/s to about 0.25 cm/s. The decrease in e′ value may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of CK-274, or a pharmaceutically acceptable salt thereof, is administered to a patient with non-obstructive hypertrophic cardiomyopathy, thereby decreasing a lateral E/e′ ratio for the patient. In response to administration of the therapeutically effective amount of CK-274, or a pharmaceutically acceptable salt thereof, the lateral E/e′ ratio can decrease by about 0.5 or more, by 1 or more, by about 1.2 or more, by about 1.5 or more, or by about 1.8 or more. In some embodiments, in response to administration of the therapeutically effective amount of CK-274, or a pharmaceutically acceptable salt thereof, the lateral E/e′ ratio decreases by about 0.5 to about 2, such as by about 1 to about 1.8, or about 1.5 to about 1.8. The decrease in lateral E/e′ ratio may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of CK-274, or a pharmaceutically acceptable salt thereof, is administered to a patient with non-obstructive hypertrophic cardiomyopathy, thereby decreasing a likelihood of systolic anterior motion (SAM) of the mitral valve leaflet for the patient.

In some embodiments, a therapeutically effective amount of CK-274, or a pharmaceutically acceptable salt thereof, is administered to a patient with non-obstructive hypertrophic cardiomyopathy, thereby decreasing a level of brain natriuretic peptide or N-terminal prohormone of brain natriuretic peptide (NT-proBNP) in the patient. The decrease in a level of brain natriuretic peptide or N-terminal prohormone of brain natriuretic peptide (NT-proBNP) in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments, a therapeutically effective amount of CK-274, or a pharmaceutically acceptable salt thereof, is administered to a patient with non-obstructive hypertrophic cardiomyopathy, thereby decreasing a level of cardiac troponin I. The decrease in a level of cardiac troponin I in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments of any of the foregoing, the patient with non-obstructive hypertrophic cardiomyopathy is classified as NYHA class III when administration with CK-274, or a pharmaceutically acceptable salt thereof, is initiated. In some embodiments, the patient with non-obstructive hypertrophic cardiomyopathy is classified as NYHA class II when administration with CK-274, or a pharmaceutically acceptable salt thereof, is initiated.

In some embodiments of any of the methods disclosed herein, the methods result in a reduction in the patient's NYHA classification by one or more classes, and/or a decrease in NT-proBNP, and/or a decrease in the level of troponin I (e.g., cardiac troponin I). In some embodiments, the methods result in a reduction in the patient's NYHA classification by one or more classes, or a decrease in NT-proBNP, or a decrease in the level of troponin I (e.g., cardiac troponin I). In some embodiments, the methods result in a reduction in the patient's NYHA classification by one or more classes, and a decrease in NT-proBNP. In some embodiments, the methods result in a reduction in the patient's NYHA classification by one or more classes, and a decrease in the level of troponin I (e.g., cardiac troponin I). In some embodiments, the methods result in a decrease in NT-proBNP and a decrease in the level of troponin I (e.g., cardiac troponin I). In some embodiments, the methods result in a reduction in the patient's NYHA classification by one or more classes, a decrease in NT-proBNP, and a decrease in the level of troponin I (e.g., cardiac troponin I).

In some embodiments of any of the foregoing, administration of CK-274, or a pharmaceutically acceptable salt thereof, to a patient with non-obstructive hypertrophic cardiomyopathy further results in decreased left ventricular wall stress and/or decreased myocardial injury in the patient. The decrease in left ventricular wall stress and/or the decrease in myocardial injury in the patient may occur after about 1 week, after about 2 weeks, after about 3 weeks, after about 4 weeks, after about 5 weeks, after about 6 weeks, after about 8 weeks, or after about 10 weeks of daily dose administration.

In some embodiments of any of the foregoing, administration of CK-274, or a pharmaceutically acceptable salt thereof, to a patient with non-obstructive hypertrophic cardiomyopathy results in an improvement in patient health status. In some embodiments of any of the foregoing, administration of CK-274, or a pharmaceutically acceptable salt thereof, to a patient with hypertrophic cardiomyopathy results in an improvement in patient health status as determined by changes in the Short Form 36 physical function sub-scale (SF-36-PFS). In some embodiments of any of the foregoing, administration of CK-274, or a pharmaceutically acceptable salt thereof, to a patient with hypertrophic cardiomyopathy results in an improvement in patient health status as determined by changes in the Kansas City Cardiomyopathy Questionnaire-Clinical Summary Score (KCCQ-CSS). In some embodiments, administration of CK-274, or a pharmaceutically acceptable salt thereof, to a patient with hypertrophic cardiomyopathy results in improvement in KCCQ-CSS by 1 point, 2 points, 3 points, 4 points, 5 points, or more than 5 points. In some embodiments of any of the foregoing, administration of CK-274, or a pharmaceutically acceptable salt thereof, to a patient with hypertrophic cardiomyopathy results in an improvement in patient health status and health-related quality of life as measured by PRO questionnaire, as determined by changes from responses to the EuroQol 5-dimension 5-level instrument (EQ-5D-5L). In some embodiments of any of the foregoing, administration of CK-274, or a pharmaceutically acceptable salt thereof, to a patient with hypertrophic cardiomyopathy results in an improvement in patient health status as determined by changes in Seattle Angina Questionnaire-Angina Frequency (SAQ-AF). In some embodiments of any of the foregoing, administration of CK-274, or a pharmaceutically acceptable salt thereof, to a patient with hypertrophic cardiomyopathy results in an improvement in patient health status as determined by changes in Seattle Angina Questionnaire-7 (SAQ-7).

In some embodiments, administration with CK-274, or a pharmaceutically acceptable salt thereof, to a patient with non-obstructive hypertrophic cardiomyopathy results in a sustained effect for at least 10 weeks, 12 weeks, 6 months, 1 year, 2 years, 3 years, 4 years, or 5 years. In some embodiments, administration with CK-274, or a pharmaceutically acceptable salt thereof, to a patient with non-obstructive hypertrophic cardiomyopathy results in a sustained effect for at least 6 months. In some embodiments, administration with CK-274, or a pharmaceutically acceptable salt thereof, to a patient with hypertrophic cardiomyopathy results in a sustained effect for at least 1 year. In some embodiments, administration with CK-274, or a pharmaceutically acceptable salt thereof, to a patient with non-obstructive hypertrophic cardiomyopathy results in a sustained effect for at least 5 years.

Administration of the compounds and compositions disclosed and/or described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

CK-274 can be administered either alone or in combination with one or more conventional pharmaceutical carriers or excipients (e.g., mannitol, lactose, starch, magnesium stearate, sodium saccharine, talcum, cellulose, sodium croscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pennsylvania. Suitable formulations of CK-274 are disclosed in WO 2021/011808, which is incorporated by reference herein in its entirety.

In some embodiments, CK-274 is provided in a formulation comprising: (i) CK-274 or a pharmaceutically acceptable salt thereof; (ii) a filler; (iii) a binder; (iv) a disintegrant; (v) a surfactant; and (vi) a lubricant. In some embodiments, CK-274 is provided in a formulation comprising: (i) CK-274; (ii) a filler; (iii) a binder; (iv) a disintegrant; (v) a surfactant; and (vi) a lubricant.

In some embodiments, the filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrines, and mixtures of any of the foregoing.

In some embodiments, the binder is selected from the group consisting of arabic gum, acacia gum, alginate, alginic acid, corn starch, copolyvidone, polyvinylpyrrolidone, gelatin, glyceryl behenate, hydroxyethylcellulose, hydroxypropyl cellulose, carboxymethylcellulose, carboxymethylcellulose calcium, carboxymethylcellulose sodium, methylcellulose, hypromellose, lactose, polyvinyl alcohol, povidone, polyethylene oxide, polyacrylates, potato starch, pregelatinized starch, sodium alginate, sodium starch, sodium carboxy methyl cellulose, starch, and mixtures of any of the foregoing.

In some embodiments, the disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the surfactant is selected from the group consisting of cetylpyridine chloride, heptadecaethylene oxycetanol, lecithins, polyoxyethylene stearate, nonoxynol 9, nonoxynol 10, octoxynol 9, sorbitan fatty acid esters, Span 20, Span 40, Span 60, Span 80, Span 85, polysorbates, polysorbate 20, polysorbate 21, polysorbate 40, polysorbate 60, polysorbate 61, polysorbate 65, polysorbate 80, sodium salts of fatty alcohol sulfates, sodium lauryl sulfate, sodium salts of sulfosuccinates, sodium dioctylsulfosuccinate, partial esters of fatty acids with alcohols, glycerine monostearate, glyceryl monooleate, ethers of fatty alcohols with polyoxyethylene, esters of fatty acids with polyoxyethylene, copolymers of ethylenoxide and propylenoxide (Pluronic®), benzalkonium chloride, ethoxylated triglycerides, and mixtures of any of the foregoing.

In some embodiments, the lubricant is selected from the group consisting of hydrogenated castor oil, magnesium stearate, glyceryl monostearate, calcium stearate, glyceryl behenate, glycerol distearate, glyceryl dipalmitostearate, behenoyl polyoxyl-8 glycerides, sodium stearyl fumarate, stearic acid, talc, zinc stearate, mineral oil, polyethylene glycol, polaxamer, sodium lauryl sulfate, and mixtures of any of the foregoing.

In some embodiments, the formulation comprises: (i) about 1% by weight to about 80% by weight of the CK-274 or a pharmaceutically acceptable salt thereof; (ii) about 15% by weight to about 90% by weight of the filler; (iii) about 0.1% by weight to about 10% by weight of the binder; (iv) about 1% by weight to about 10% by weight of the disintegrant; (v) about 0.1% by weight to about 10% by weight of the surfactant; and (vi) about 0.1% by weight to about 10% by weight of the lubricant.

In some embodiments, the formulation comprises: (i) about 1% by weight to about 50% by weight of the CK-274 or pharmaceutically acceptable salt thereof; (ii) about 40% by weight to about 80% by weight of the filler; (iii) about 0.5% by weight to about 5% by weight of the binder; (iv) about 2% by weight to about 8% by weight of the disintegrant; (v) about 0.5% by weight to about 5% by weight of the surfactant; and (vi) about 0.5% by weight to about 5% by weight of the lubricant.

In some embodiments, the formulation comprises: (i) about 10% by weight to about 30% by weight of the CK-274, or pharmaceutically acceptable salt thereof; (ii) about 60% by weight to about 80% by weight of the filler; (iii) about 1% by weight to about 3% by weight of the binder; (iv) about 4% by weight to about 6% by weight of the disintegrant; (v) about 1% by weight to about 3% by weight of the surfactant; and (vi) about 0.5% by weight to about 1.5% by weight of the lubricant.

In some embodiments, the formulation comprises: (i) about 1% by weight to about 10% by weight of the CK-274, or pharmaceutically acceptable salt thereof; (ii) about 70% by weight to about 90% by weight of the filler; (iii) about 1% by weight to about 3% by weight of the binder; (iv) about 4% by weight to about 6% by weight of the disintegrant; (v) about 1% by weight to about 3% by weight of the surfactant; and (vi) about 0.5% by weight to about 1.5% by weight of the lubricant.

In some embodiments, the formulation comprises: (i) about 5% by weight of the CK-274, or pharmaceutically acceptable salt thereof; (ii) about 85% by weight of the filler; (iii) about 2% by weight of the binder; (iv) about 5% by weight of the disintegrant; (v) about 2% by weight of the surfactant; and (vi) about 1% by weight of the lubricant.

In some embodiments, the formulation comprises: (i) about 10% by weight of the CK-274, or pharmaceutically acceptable salt thereof; (ii) about 80% by weight of the filler; (iii) about 2% by weight of the binder; (iv) about 5% by weight of the disintegrant; (v) about 2% by weight of the surfactant; and (vi) about 1% by weight of the lubricant.

In some embodiments, the formulation comprises: (i) about 1% by weight to about 50% by weight of CK-274, or pharmaceutically acceptable salt thereof; (ii-1) about 10% by weight to about 60% by weight of mannitol; (ii-2) about 5% by weight to about 45% by weight of microcrystalline cellulose; (iii) about 0.1% by weight to about 10% by weight of hydroxypropyl cellulose; (iv) about 1% by weight to about 10% by weight of croscarmellose sodium; (v) about 0.1% by weight to about 10% by weight of sodium lauryl sulfate; and (vi) about 0.1% by weight to about 10% by weight of magnesium stearate.

In some embodiments, the formulation comprises: (i) about 10% by weight to about 30% by weight of the CK-274, or pharmaceutically acceptable salt thereof; (ii-1) about 40% by weight to about 50% by weight of mannitol; (ii-2) about 20% by weight to about 30% by weight of microcrystalline cellulose; (iii) about 1% by weight to about 3% by weight of hydroxypropyl cellulose; (iv) about 4% by weight to about 6% by weight of croscarmellose sodium; (v) about 1% by weight to about 3% by weight of sodium lauryl sulfate; and (vi) about 0.5% by weight to about 1.5% by weight of magnesium stearate.

In some embodiments, the formulation comprises: (i) about 1% by weight to about 10% by weight of CK-274, or pharmaceutically acceptable salt thereof; (ii-1) about 50% by weight to about 60% by weight of mannitol; (ii-2) about 25% by weight to about 35% by weight of microcrystalline cellulose; (iii) about 1% by weight to about 3% by weight of hydroxypropyl cellulose; (iv) about 4% by weight to about 6% by weight of croscarmellose sodium; (v) about 1% by weight to about 3% by weight of sodium lauryl sulfate; and (vi) about 0.5% by weight to about 1.5% by weight of the magnesium stearate.

In some embodiments, the formulation comprises: (i) about 20% by weight of the CK-274, or pharmaceutically acceptable salt thereof; (ii-1) about 44% by weight of mannitol; (ii-2) about 26% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of magnesium stearate.

In some embodiments, the formulation comprises: (i) about 10% by weight of the CK-274, or pharmaceutically acceptable salt thereof; (ii-1) about 50% by weight of mannitol; (ii-2) about 30% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of magnesium stearate.

In some embodiments, the formulation comprises: (i) about 5% by weight of the CK-274, or pharmaceutically acceptable salt thereof; (ii-1) about 54% by weight of mannitol; (ii-2) about 31% by weight of microcrystalline cellulose; (iii) about 2% by weight of hydroxypropyl cellulose; (iv) about 5% by weight of croscarmellose sodium; (v) about 2% by weight of sodium lauryl sulfate; and (vi) about 1% by weight of the magnesium stearate.

In some embodiments, the aforementioned formulations are in the form of a tablet. In some embodiments, the tablet further comprises a coating (e.g., a film coating as described elsewhere herein). In such embodiments, the weight percentages herein are provided with respect to the core tablet, not including the weight of the coating.

In some embodiments, CK-274 or pharmaceutical composition containing CK-274 will take the form of a pill or tablet and thus the composition may contain, along with compounds disclosed and/or described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

In some embodiments, CK-274 is provided in a tablet comprising: (i) a core having a total core weight comprising: (a) an intra-granular component comprising: (a-i) CK-274, or pharmaceutically acceptable salt thereof; (a-ii) an intra-granular filler; (a-iii) an intra-granular binder; (a-iv) an intra-granular disintegrant; and (a-v) an intra-granular surfactant; and (b) an extra-granular component comprising: (b-i) an extra-granular filler; (b-ii) an extra-granular disintegrant; and (b-iii) an extra-granular lubricant; and optionally (ii) a coating layer comprising a coating agent.

In some embodiments, the intra-granular filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrines, and mixtures of any of the foregoing.

In some embodiments, the intra-granular binder is selected from the group consisting of arabic gum, acacia gum, alginate, alginic acid, corn starch, copolyvidone, polyvinylpyrrolidone, gelatin, glyceryl behenate, hydroxyethylcellulose, hydroxypropyl cellulose, carboxymethylcellulose, carboxymethylcellulose calcium, carboxymethylcellulose sodium, methylcellulose, hypromellose, lactose, polyvinyl alcohol, povidone, polyethylene oxide, polyacrylates, potato starch, pregelatinized starch, sodium alginate, sodium starch, sodium carboxy methyl cellulose, starch, and mixtures of any of the foregoing.

In some embodiments, the intra-granular disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the intra-granular surfactant is selected from the group consisting of cetylpyridine chloride, heptadecaethylene oxycetanol, lecithins, polyoxyethylene stearate, nonoxynol 9, nonoxynol 10, octoxynol 9, sorbitan fatty acid esters, Span 20, Span 40, Span 60, Span 80, Span 85, polysorbates, polysorbate 20, polysorbate 21, polysorbate 40, polysorbate 60, polysorbate 61, polysorbate 65, polysorbate 80, sodium salts of fatty alcohol sulfates, sodium lauryl sulfate, sodium salts of sulfosuccinates, sodium dioctylsulfosuccinate, partial esters of fatty acids with alcohols, glycerine monostearate, glyceryl monooleate, ethers of fatty alcohols with polyoxyethylene, esters of fatty acids with polyoxyethylene, copolymers of ethylenoxide and propylenoxide (Pluronic®), benzalkonium chloride, ethoxylated triglycerides, and mixtures of any of the foregoing.

In some embodiments, the extra-granular filler is selected from the group consisting of powdered cellulose, microcrystalline cellulose, silicified microcrystalline cellulose, kaolin, corn starch, maize starch, starch derivatives, pregelatinized starch, calcium phosphate, calcium hydrogen phosphate, dicalcium phosphate, tricalcium phosphate, compressible sugar, sugar alcohol, mannitol, sorbitol, maltitol, xylitol, lactitol, lactose, dextrose, maltose, sucrose, glucose, fructose, saccharose, raffinose, dextrates, trehalose, maltodextrines, and mixtures of any of the foregoing.

In some embodiments, the extra-granular disintegrant is selected from the group consisting of alginic acid, croscarmellose sodium, cellulose, carboxymethylcellulose calcium, carboxymethylcellulose sodium, microcrystalline cellulose, crospovidone, sodium starch glycolate, low-substituted hydroxypropyl cellulose, polacrillin potassium, pregelatinized starch, partially hydrolyzed starch, sodium carboxymethyl starch, starch, sodium alginate, sodium carboxy methyl cellulose, and mixtures of any of the foregoing.

In some embodiments, the extra-granular lubricant is selected from the group consisting of hydrogenated castor oil, magnesium stearate, glyceryl monostearate, calcium stearate, glyceryl behenate, glycerol distearate, glyceryl dipalmitostearate, behenoyl polyoxyl-8 glycerides, sodium stearyl fumarate, stearic acid, talc, zinc stearate, mineral oil, polyethylene glycol, polaxamer, sodium lauryl sulfate, and mixtures of any of the foregoing.

In some embodiments, the core comprises: (a) an intra-granular component comprising: (a-i) about 1% to about 80% of the total core weight of the CK-274, or pharmaceutically acceptable salt thereof; (a-ii) about 10% to about 80% of the total core weight of the intra-granular filler; (a-iii) about 0.1% to about 10% of the total core weight of the intra-granular binder; (a-iv) about 0.1% to about 5% of the total core weight of the intra-granular disintegrant; and (a-v) about 0.1% to about 5% of the total core weight of the intra-granular surfactant; and (b) an extra-granular component comprising: (b-i) about 5% to about 15% of the total core weight of the extra-granular filler; (b-ii) about 0.1% to about 5% of the total core weight of the extra-granular disintegrant; and (b-iii) about 0.1% to about 5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 80% of the total core weight of the CK-274, or pharmaceutically acceptable salt thereof; (a-ii) about 10% to about 80% of the total core weight of the intra-granular filler; (a-iii) about 0.1% to about 10% of the total core weight of the intra-granular binder; (a-iv) about 0.1% to about 5% of the total core weight of the intra-granular disintegrant; and (a-v) about 0.1% to about 5% of the total core weight of the intra-granular surfactant; and (b) an extra-granular component comprising: (b-i) about 5% to about 15% of the total core weight of the extra-granular filler; (b-ii) about 0.1% to about 5% of the total core weight of the extra-granular disintegrant; and (b-iii) about 0.1% to about 5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 50% of the total core weight of the CK-274, or pharmaceutically acceptable salt thereof; (a-ii) about 40% to about 80% of the total core weight of the intra-granular filler; (a-iii) about 1% to about 5% of the total core weight of the intra-granular binder; (a-iv) about 1% to about 5% of the total core weight of the intra-granular disintegrant; and (a-v) about 1% to about 5% of the total core weight of the intra-granular surfactant; and (b) an extra-granular component comprising: (b-i) about 5% to about 15% of the total core weight of the extra-granular filler; (b-ii) about 1% to about 5% of the total core weight of the extra-granular disintegrant; and (b-iii) about 0.1% to about 2% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% to about 30% of the total core weight of the CK-274, or pharmaceutically acceptable salt thereof; (a-ii) about 50% to about 70% of the total core weight of the intra-granular filler; (a-iii) about 1% to about 3% of the total core weight of the intra-granular binder; (a-iv) about 2% to about 4% of the total core weight of the intra-granular disintegrant; and (a-v) about 1% to about 3% of the total core weight of the intra-granular surfactant; and (b) an extra-granular component comprising: (b-i) about 5% to about 15% of the total core weight of the an extra-granular filler; (b-ii) about 1% to about 3% of the total core weight of the extra-granular disintegrant; and (b-iii) about 0.1% to about 1.5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 10% of the total core weight of the CK-274, or pharmaceutically acceptable salt thereof; (a-ii) about 60% to about 80% of the total core weight of the intra-granular filler; (a-iii) about 1% to about 3% of the total core weight of the intra-granular binder; (a-iv) about 2% to about 4% of the total core weight of the intra-granular disintegrant; and (a-v) about 1% to about 3% of the total core weight of the intra-granular surfactant; and (b) an extra-granular component comprising: (b-i) about 5% to about 15% of the total core weight of the an extra-granular filler; (b-ii) about 1% to about 3% of the total core weight of the extra-granular disintegrant; and (b-iii) about 0.1% to about 1.5% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 5% of the total core weight of the CK-274, or pharmaceutically acceptable salt thereof; (a-ii) about 74% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% of the total core weight of the CK-274, or pharmaceutically acceptable salt thereof; (a-ii) about 69% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 20% of the total core weight of the CK-274, or pharmaceutically acceptable salt thereof; (a-ii) about 59% of the total core weight of the intra-granular filler; (a-iii) about 2% of the total core weight of the intra-granular binder; (a-iv) about 3% of the total core weight of the intra-granular disintegrant; and (a-v) about 2% of the total core weight of the intra-granular surfactant; and (b) an extra-granular component comprising: (b-i) about 11% of the total core weight of the an extra-granular filler; (b-ii) about 2% of the total core weight of the extra-granular disintegrant; and (b-iii) about 1% of the total core weight of the extra-granular lubricant. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% to about 50% of the total core weight of the CK-274, or pharmaceutically acceptable salt thereof; (a-ii-1) about 40% to about 60% of the total core weight of mannitol; (a-ii-2) about 10% to about 30% of the total core weight of microcrystalline cellulose; (a-iii) about 1% to about 5% of the total core weight of hydroxypropyl cellulose; (a-iv) about 1% to about 5% of the total core weight of croscarmellose sodium; and (a-v) about 1% to about 5% of the total core weight of sodium lauryl sulfate; and (b) an extra-granular component comprising: (b-i) about 5% to about 15% of the total core weight of microcrystalline cellulose; (b-ii) about 1% to about 5% of the total core weight of croscarmellose sodium; and (b-iii) about 0.1% to about 2% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% to about 30% of the total core weight of the CK-274, or pharmaceutically acceptable salt thereof; (a-ii-1) about 40% to about 50% of the total core weight of mannitol; (a-ii-2) about 10% to about 20% of the total core weight of microcrystalline cellulose; (a-iii) about 1% to about 3% of the total core weight of hydroxypropyl cellulose; (a-iv) about 2% to about 4% of the total core weight of croscarmellose sodium; and (a-v) about 1% to about 3% of the total core weight of sodium lauryl sulfate; and (b) an extra-granular component comprising: (b-i) about 5% to about 15% of the total core weight of microcrystalline cellulose; (b-ii) about 1% to about 3% of the total core weight of croscarmellose sodium; and (b-iii) about 0.1% to about 1.5% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 1% by weight to about 10% of the total core weight of the CK-274, or pharmaceutically acceptable salt thereof; (a-ii-1) about 50% to about 60% of the total core weight of mannitol; (a-ii-2) about 15% to about 25% of the total core weight of microcrystalline cellulose; (a-iii) about 1% to about 3% of the total core weight of hydroxypropyl cellulose; (a-iv) about 2% to about 4% of the total core weight of croscarmellose sodium; and (a-v) about 1% to about 3% of the total core weight of sodium lauryl sulfate; and (b) an extra-granular component comprising: (b-i) about 5% to about 15% of the total core weight of microcrystalline cellulose; (b-ii) about 1% to about 3% of the total core weight of croscarmellose sodium; and (b-iii) about 0.1% to about 1.5% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 20% of the total core weight of the CK-274, or pharmaceutically acceptable salt thereof; (a-ii-1) about 44% of the total core weight of mannitol; (a-ii-2) about 15% of the total core weight of microcrystalline cellulose; (a-iii) about 2% of the total core weight of hydroxypropyl cellulose; (a-iv) about 3% of the total core weight of croscarmellose sodium; and (a-v) about 2% of the total core weight of sodium lauryl sulfate; and (b) an extra-granular component comprising: (b-i) about 11% of the total core weight of microcrystalline cellulose; (b-ii) about 2% of the total core weight of croscarmellose sodium; and (b-iii) about 1% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg.

In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 10% of the total core weight of the CK-274, or pharmaceutically acceptable salt thereof; (a-ii-1) about 50% of the total core weight of mannitol; (a-ii-2) about 19% of the total core weight of microcrystalline cellulose; (a-iii) about 2% of the total core weight of hydroxypropyl cellulose; (a-iv) about 3% of the total core weight of croscarmellose sodium; and (a-v) about 2% of the total core weight of sodium lauryl sulfate; and (b) an extra-granular component comprising: (b-i) about 11% of the total core weight of microcrystalline cellulose; (b-ii) about 2% of the total core weight of croscarmellose sodium; and (b-iii) about 1% of the total core weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg.

In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the core comprises: (a) an intra-granular component comprising: (a-i) about 5% by weight of the CK-274, or pharmaceutically acceptable salt thereof; (a-ii-1) about 54% by weight of mannitol; (a-ii-2) about 20% by weight of microcrystalline cellulose; (a-iii) about 2% by weight of hydroxypropyl cellulose; (a-iv) about 3% by weight of croscarmellose sodium; and (a-v) about 2% by weight of sodium lauryl sulfate; and (b) an extra-granular component comprising: (b-i) about 11% by weight of microcrystalline cellulose; (b-ii) about 2% by weight of croscarmellose sodium; and (b-iii) about 1% by weight of extra-granular lubricant. In some embodiments, the extra-granular lubricant is magnesium stearate. In some embodiments, the total core weight is about 50 mg, about 70 mg, about 100 mg, about 150 mg, about 200 mg, or about 400 mg. In some embodiments, the total core weight is about 50 mg, about 100 mg, about 150 mg, or about 200 mg. In some embodiments, the total core weight is about 50 mg. In some embodiments, the total core weight is about 70 mg. In some embodiments, the total core weight is about 100 mg. In some embodiments, the total core weight is about 150 mg. In some embodiments, the total core weight is about 200 mg. In some embodiments, the total core weight is about 400 mg.

In some embodiments, the tablet comprises a coating layer comprising a coating agent. In some embodiments, the coating layer surrounds the total core of the tablet. In some embodiments, the coating agent is selected from the group consisting of Opadry QX White 21A180025, Opadry I, and Opadry II. In some embodiments, the coating agent is Opadry QX White 21A180025. In some embodiments, the tablet comprises about 0.5% to about 10% of the total core weight of coating agent. In some embodiments, the tablet comprises about 1% to about 5% of the total core weight of coating agent. In some embodiments, the tablet comprises about 2% to about 4% of the total core weight of coating agent. In some embodiments, the tablet comprises about 3% of the total core weight of coating agent. In some embodiments, the tablet comprises about 0.5% to about 10% of the total core weight of Opadry QX White 21A180025. In some embodiments, the tablet comprises about 1% to about 5% of the total core weight of Opadry QX White 21A180025. In some embodiments, the tablet comprises about 2% to about 4% of the total core weight of Opadry QX White 21A180025. In some embodiments, the tablet comprises about 3% of the total core weight of Opadry QX White 21A180025.

In some embodiments, the amount of CK-274 (which may exist in various forms) in a formulation, e.g., a tablet, is about a daily dose as described herein, e.g., of about 5 mg, about 10 mg, about 15 mg, or about 20 mg of CK-274. In some embodiments, the amount of CK-274 (which may exist in various forms) in a formulation (e.g., a tablet) is about half of a daily dose as described herein. In some embodiments, the amount of CK-274 in a formulation (e.g., a tablet) is about 2.5 mg, about 5 mg, about 7.5 mg, or about 10 mg of CK-274.

In some embodiments, CK-274 exists in its free base form in various formulations, e.g., tablets. In some embodiments, a method, e.g., a method described in an Example, uses such tablets.

Liquid pharmaceutically administrable compositions can, for example, be prepared by dissolving, dispersing or suspending etc. a compound disclosed and/or described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compound contained in such parenteral compositions depends, for example, on the physical nature of the compound, the activity of the compound and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound disclosed and/or described herein in solution.

Pharmaceutical compositions of the compounds disclosed and/or described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns.

In addition, pharmaceutical compositions can include a compound disclosed and/or described herein and one or more additional medicinal agents, pharmaceutical agents, adjuvants, and the like. Suitable medicinal and pharmaceutical agents include those described herein.

Treatment of Hypertrophic Cardiomyopathy with Mid-Ventricular Obstruction

In some embodiments of any of the foregoing methods, the non-obstructive HCM is HCM with mid-ventricular obstruction (MVO). For instance, in some embodiments, the methods disclosed herein comprise administering a therapeutically effective amount of CK-274 to a patient for the treatment of hypertrophic cardiomyopathy with mid-ventricular obstruction (MVO), as described herein for the treatment of non-obstructive hypertrophic cardiomyopathy. For instance, in some embodiments, the patient in need thereof has HCM with mid-ventricular obstruction. It is to be understood that the term “mid-ventricular obstruction” (MVO) may be used interchangeably with “mid-cavitary obstruction” in the embodiments disclosed herein.

### Reducing Angina Frequency

In some embodiments, the present disclosure provides methods for reducing angina frequency in a subject as described herein, comprising administering or delivering to a subject CK-274 as described herein. In some embodiments, a subject is a human subject suffering from non-obstructive hypertrophic cardiomyopathy. In some embodiments, CK-274 is administered or delivered as a free base. In some embodiments, CK-274 is administered or delivered as a pharmaceutically acceptable salt. In some embodiments, CK-274 is administered as a tablet, e.g., a tablet comprising about a daily dose, or half of a daily dose, as described herein. In some embodiments, a daily dose is about 5 mg, about 10 mg, about 15 mg or about 20 mg. In some embodiments, a daily dose is about 10 mg. In some embodiments, a daily dose is about 15 mg. In some embodiments, a daily dose is about 20 mg.

### Polymorphs

In some embodiments, CK-274 is in a polymorph form of (R)—N-(5-(5-ethyl-1,2,4-oxadiazol-3-yl)-2,3-dihydro-1H-inden-1-yl)-1-methyl-1H-pyrazole-4-carboxamide. Suitable polymorphs of CK-274 include those disclosed in and may be prepared according to WO 2021/011807, which is incorporated by reference herein in its entirety. The polymorphs may have properties such as bioavailability and stability under certain conditions that are suitable for medical or pharmaceutical uses. Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, polymorphs may provide desired or suitable hygroscopicity, particle size control, dissolution rate, solubility, purity, physical and chemical stability, manufacturability, yield, reproducibility, and/or process control. Thus, polymorphs of (R)—N-(5-(5-ethyl-1,2,4-oxadiazol-3-yl)-2,3-dihydro-1H-inden-1-yl)-1-methyl-1H-pyrazole-4-carboxamide may provide advantages of improving the manufacturing process of an active agent or the stability or storability of a drug product form of the active agent, or having suitable bioavailability and/or stability as an active agent. In some embodiments, the CK-274 in a formulation, e.g., a tablet, is one of polymorphic Form I, Form II, Form III, Form IV, Form V, or Form VI, or a combination thereof. In some embodiments, the CK-274 is one of polymorphic Form I, Form II, Form III, Form IV, Form V, or Form VI. In some embodiments, the CK-274 is polymorphic Form I or Form IV. In some embodiments, the CK-274 is polymorphic Form I. In some embodiments, the CK-274 is polymorphic Form IV.

### Form I

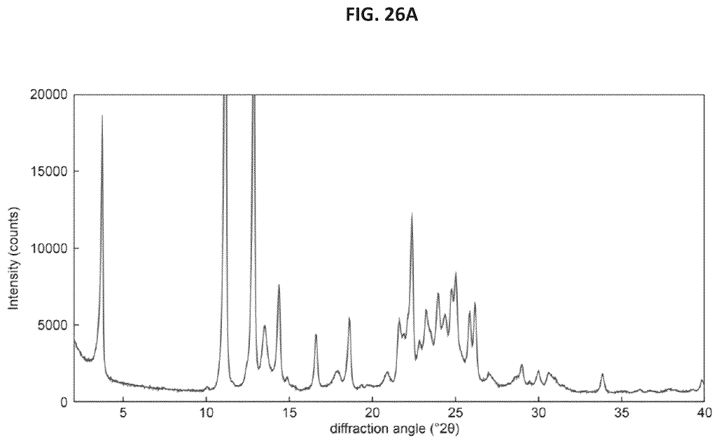

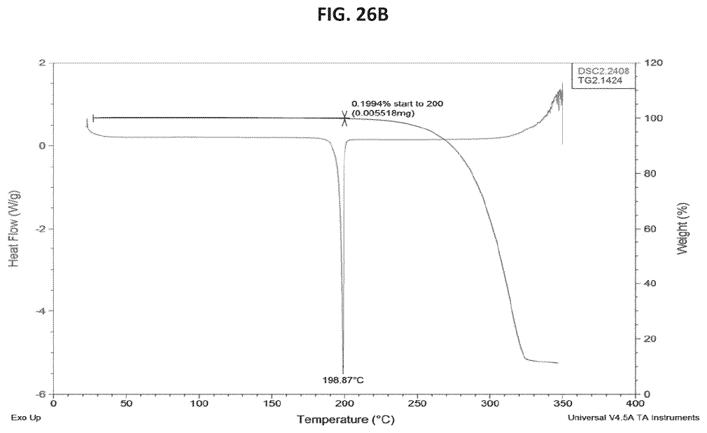

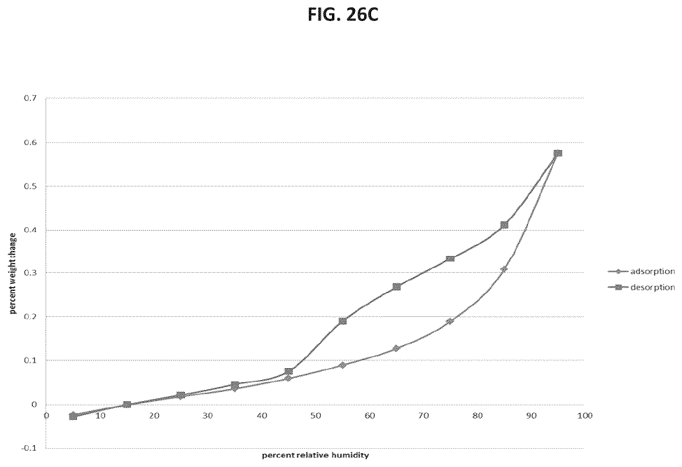

In some embodiments, the CK-274 is polymorphic Form I of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form I using XRPD are shown in Table P-1. In some embodiments, Form I has an XRPD pattern substantially as shown in FIG. 26A.

TABLE P-1

| Angle (°2θ) | d value (Å) | Intensity (counts) |
|---|---|---|
| 3.7 | 23.6 | 55228.97 |
| 11.2 | 7.9 | 126123.6 |
| 12.9 | 6.9 | 94231.09 |
| 13.5 | 6.6 | 12945.13 |
| 14.4 | 6.2 | 22543.29 |
| 14.9 | 6.0 | 1982.81 |
| 16.6 | 5.3 | 12426.88 |
| 17.8 | 5.0 | 3641.86 |
| 18.6 | 4.8 | 15100.54 |
| 21.6 | 4.1 | 10927.77 |
| 22.2 | 4.0 | 10386.49 |
| 22.4 | 4.0 | 29345.32 |
| 22.8 | 3.9 | 4094.45 |
| 23.2 | 3.8 | 9276.26 |
| 23.9 | 3.7 | 12944.35 |
| 24.4 | 3.7 | 8288.4 |
| 24.7 | 3.6 | 14182.35 |
| 25.0 | 3.6 | 16601.84 |
| 25.8 | 3.4 | 11350.3 |
| 26.1 | 3.4 | 14448.7 |
| 28.6 | 3.1 | 3088.71 |
| 29.0 | 3.1 | 4033.18 |
| 29.4 | 3.0 | 2451.42 |
| 29.9 | 3.0 | 3631.24 |
| 30.6 | 2.9 | 4172.12 |
| 33.8 | 2.6 | 3752.58 |
| 36.1 | 2.5 | 543.33 |
| 36.8 | 2.4 | 405.69 |
| 37.8 | 2.4 | 482.5 |
| 39.8 | 2.3 | 2685.52 |

In some embodiments, polymorphic Form I has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 26A or as provided in Table P-1. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form I, can vary by about ±0.6 degrees, ±0.4 degrees, ±0.2 degrees, or ±0.1 degrees 2-theta.

In some embodiments, polymorphic Form I has an XRPD pattern comprising peaks at angles 2-theta of 3.7±0.2, 11.2±0.2, 12.9±0.2, 13.5±0.2, 14.4±0.2, 14.9±0.2, 16.6±0.2, 17.8±0.2, 18.6±0.2, 21.6±0.2, 22.2±0.2, 22.4±0.2, 22.8±0.2, 23.2±0.2, 23.9±0.2, 24.4±0.2, 24.7±0.2, 25.0±0.2, 25.8±0.2, 26.1±0.2, 28.6±0.2, 29.0±0.2, 29.4±0.2, 29.9±0.2, 30.6±0.2, 33.8±0.2, 36.1±0.2, 36.8±0.2, 37.8±0.2, and 39.8±0.2 degrees. In some embodiments, polymorphic Form I has an XRPD pattern comprising peaks at angles 2-theta of 3.7±0.2, 11.2±0.2, 12.9±0.2, 13.5±0.2, 14.4±0.2, 18.6±0.2, 22.4±0.2, 24.7±0.2, 25.0±0.2, and 26.1±0.2 degrees. In some embodiments, polymorphic Form I has an XRPD pattern comprising peaks at angles 2-theta of 3.7±0.2, 11.2±0.2, 12.9±0.2, 14.4±0.2, and 22.4±0.2 degrees. It is to be understood that additional peaks in the XRPD pattern other than those shown in FIG. 26A or as provided in Table P-1 may be observed, for instance, due to the presence of impurities, solvent, or other polymorphs or amorphic forms present in the test sample.

In some embodiments, Form I has a differential DSC graph substantially as shown in FIG. 26B. In some embodiments, Form I is characterized as having an endotherm onset at about 199° C. as determined by DSC. In some embodiments, Form I is characterized as having an endotherm onset at 199±2° C. (e.g., 199±1.9° C., 199±1.8° C., 199±1.7° C., 199±1.6° C., 199±1.5° C., 199±1.4° C., 199±1.3° C., 192±1.2° C., 199±1, 199±0.9° C., 199±0.8° C., 199±0.7° C., 199±0.6° C., 199±0.5° C., 199±0.4° C., 199±0.3° C., 199±0.2° C., or 199±0.1° C.) as determined by DSC.

In some embodiments, Form I has a TGA graph substantially as shown in FIG. 26B.

In some embodiments, Form I has a DVS graph substantially as shown in FIG. 26C.

In some embodiments of Form I, at least one, at least two, at least three, at least four, at least five, or all of the following (a)-(f) apply:
(a) Form I has an XRPD pattern comprising peaks at angles 2-theta of 3.7±0.2, 11.2±0.2, 12.9±0.2, 14.4±0.2, and 22.4±0.2 degrees; an XRPD pattern comprising peaks at angles 2-theta of 3.7±0.2, 11.2±0.2, 12.9±0.2, 13.5±0.2, 14.4±0.2, 18.6±0.2, 22.4±0.2, 24.7±0.2, 25.0±0.2, and 26.1±0.2 degrees; or an XRPD pattern comprising peaks at angles 2-theta of 3.7±0.2, 11.2±0.2, 12.9±0.2, 13.5±0.2, 14.4±0.2, 14.9±0.2, 16.6±0.2, 17.8±0.2, 18.6±0.2, 21.6±0.2, 22.2±0.2, 22.4±0.2, 22.8±0.2, 23.2±0.2, 23.9±0.2, 24.4±0.2, 24.7±0.2, 25.0±0.2, 25.8±0.2, 26.1±0.2, 28.6±0.2, 29.0±0.2, 29.4±0.2, 29.9±0.2, 30.6±0.2, 33.8±0.2, 36.1±0.2, 36.8±0.2, 37.8±0.2, and 39.8±0.2 degrees;
(b) Form I has an XRPD pattern substantially as shown in FIG. 26A;
(c) Form I has a DSC graph substantially as shown in FIG. 26B;
(d) Form I is characterized as having an endotherm onset at about 199° C. as determined by DSC;
(e) Form I has a TGA graph substantially as shown in FIG. 26B; and
(f) Form I has a DVS graph substantially as shown in FIG. 26C.

### Form II

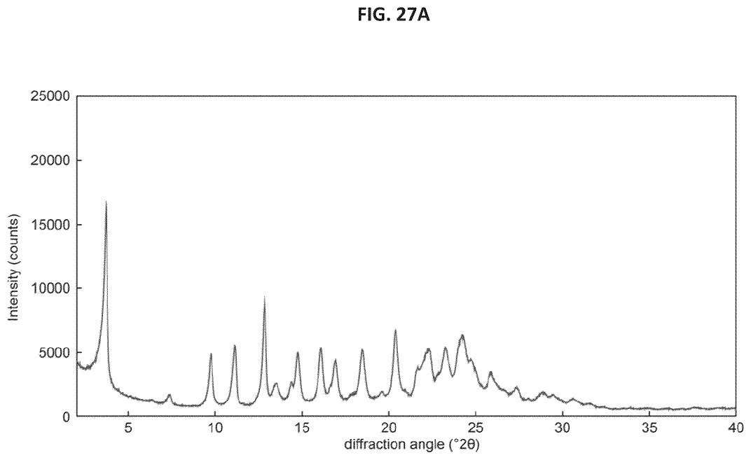

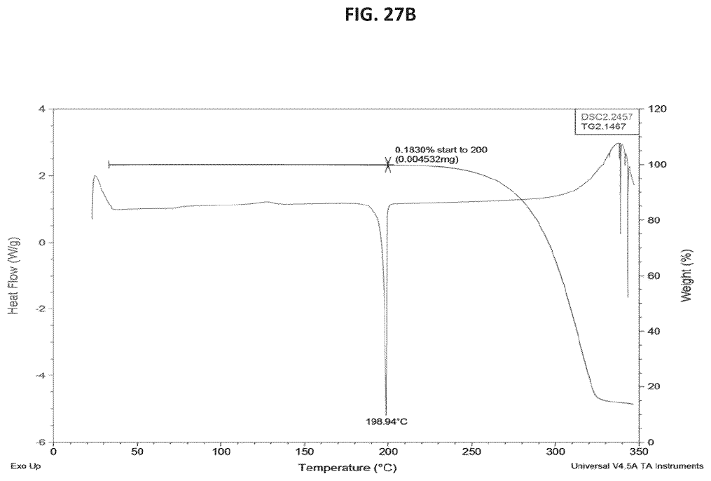

In some embodiments, the CK-274 is polymorphic Form II of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form II using XRPD are shown in Table P-2. In some embodiments, Form II has an XRPD pattern substantially as shown in FIG. 27A.

TABLE P-2

| Angle (°2θ) | d value (Å) | Intensity (counts) |
|---|---|---|
| 3.7 | 23.6 | 46289.09 |
| 7.4 | 12.0 | 2626.2 |
| 9.8 | 9.0 | 13093.89 |
| 11.1 | 7.9 | 15067.72 |
| 12.8 | 6.9 | 24560.2 |
| 13.5 | 6.6 | 4597.35 |
| 14.4 | 6.2 | 4359.38 |
| 14.7 | 6.0 | 12272.37 |
| 16.1 | 5.5 | 12841.63 |
| 17.0 | 5.2 | 8725.7 |
| 18.5 | 4.8 | 11802.76 |
| 20.4 | 4.4 | 16037.66 |
| 21.6 | 4.1 | 4907.93 |
| 22.3 | 4.0 | 10307.6 |
| 23.3 | 3.8 | 8940.83 |
| 24.0 | 3.7 | 6680.21 |
| 24.3 | 3.7 | 7959.39 |
| 24.8 | 3.6 | 6336.24 |
| 25.8 | 3.4 | 4019.98 |
| 27.4 | 3.3 | 1872.66 |
| 28.8 | 3.1 | 1783 |
| 29.5 | 3.0 | 1203.02 |
| 30.5 | 2.9 | 1080.7 |

In some embodiments, polymorphic Form II has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 27A or as provided in Table P-2. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form II, can vary by about ±0.6 degrees, ±0.4 degrees, ±0.2 degrees, or ±0.1 degrees 2-theta.

In some embodiments, polymorphic Form II has an XRPD pattern comprising peaks at angles 2-theta of 3.7±0.2, 7.4±0.2, 9.8±0.2, 11.1±0.2, 12.8±0.2, 13.5±0.2, 14.4±0.2, 14.7±0.2, 16.1±0.2, 17.0±0.2, 18.5±0.2, 20.4±0.2, 21.6±0.2, 22.3±0.2, 23.3±0.2, 24.0±0.2, 24.3±0.2, 24.8±0.2, 25.8±0.2, 27.4±0.2, 28.8±0.2, 29.5±0.2, and 30.5±0.2 degrees. In some embodiments, polymorphic Form II has an XRPD pattern comprising peaks at angles 2-theta of 3.7±0.2, 9.8±0.2, 11.1±0.2, 12.8±0.2, 14.7±0.2, 16.1±0.2, 18.5±0.2, 20.4±0.2, 22.3±0.2, and 23.3±0.2 degrees. In some embodiments, polymorphic Form II has an XRPD pattern comprising peaks at angles 2-theta of 3.7±0.2, 9.8±0.2, 11.1±0.2, 12.8±0.2, and 20.4±0.2 degrees. It is to be understood that additional peaks in the XRPD pattern other than those shown in FIG. 27A or as provided in Table P-2 may be observed, for instance, due to the presence of impurities, solvent, or other polymorphs or amorphic forms present in the test sample.

In some embodiments, Form II has a DSC graph substantially as shown in FIG. 27B. In some embodiments, Form II is characterized as having an endotherm onset at about 199° C. as determined by DSC. In some embodiments, Form II is characterized as having an endotherm onset at about 199±2° C. (e.g., 199±1.9° C., 199±1.8° C., 199±1.7° C., 199±1.6° C., 199±1.5° C., 199±1.4° C., 199±1.3° C., 199±1.2° C., 199±1, 199±0.9° C., 199±0.8° C., 199±0.7° C., 199±0.6° C., 199±0.5° C., 199±0.4° C., 199±0.3° C., 199±0.2° C., or 199±0.1° C.) as determined by DSC.

In some embodiments, Form II has a TGA graph substantially as shown in FIG. 27B.

In some embodiments of Form II, at least one, at least two, at least three, at least four, or all of the following (a)-(e) apply:
(a) Form II has an XRPD pattern comprising peaks at angles 2-theta of 3.7±0.2, 9.8±0.2, 11.1±0.2, 12.8±0.2, and 20.4±0.2 degrees; an XRPD pattern comprising peaks at angles 2-theta of 3.7±0.2, 9.8±0.2, 11.1±0.2, 12.8±0.2, 14.7±0.2, 16.1±0.2, 18.5±0.2, 20.4±0.2, 22.3±0.2, and 23.3±0.2 degrees; or an XRPD pattern comprising peaks at angles 2-theta of 3.7±0.2, 7.4±0.2, 9.8±0.2, 11.1±0.2, 12.8±0.2, 13.5±0.2, 14.4±0.2, 14.7±0.2, 16.1±0.2, 17.0±0.2, 18.5±0.2, 20.4±0.2, 21.6±0.2, 22.3±0.2, 23.3±0.2, 24.0±0.2, 24.3±0.2, 24.8±0.2, 25.8±0.2, 27.4±0.2, 28.8±0.2, 29.5±0.2, and 30.5±0.2 degrees;
(b) Form II has an XRPD pattern substantially as shown in FIG. 27A;
(c) Form II has a DSC graph substantially as shown in FIG. 27B;
(d) Form II is characterized as having a melting endotherm onset at about 199° C. as determined by DSC; and
(e) Form II has a TGA graph substantially as shown in FIG. 27B.

### Form III

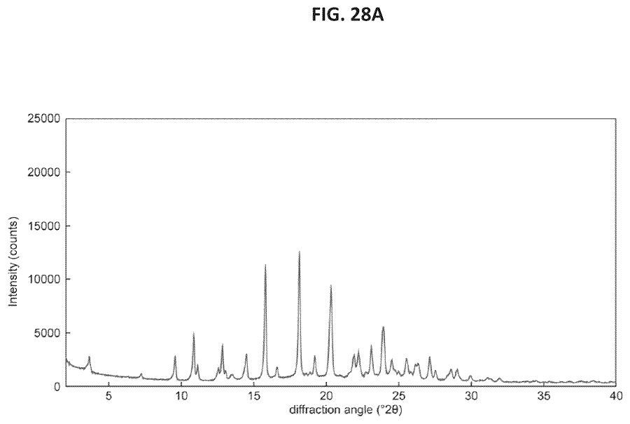

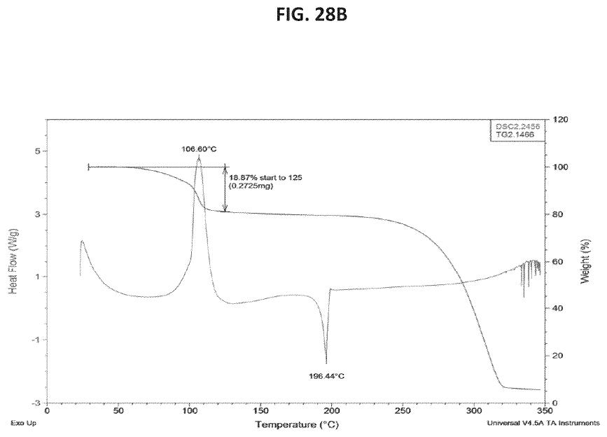

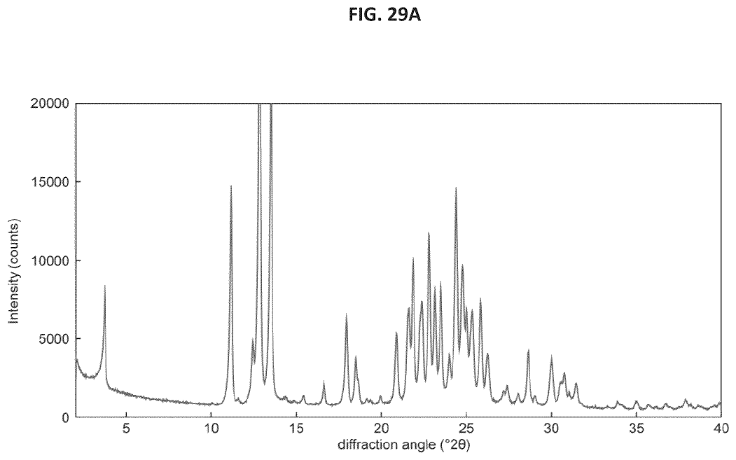

In some embodiments, the CK-274 is polymorphic Form III of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for a mixture of Forms I and III using XRPD are shown in Table P-3. In some embodiments, a mixture of Forms I and III has an XRPD pattern substantially as shown in FIG. 28A.

TABLE P-3

| Angle (°2θ) | d value (Å) | Intensity (counts) |
|---|---|---|
| 3.6 | 24.3 | 4294.42 |
| 7.2 | 12.2 | 1591.53 |
| 9.6 | 9.2 | 7874.85 |
| 10.9 | 8.1 | 14071.97 |
| 11.1 | 7.9 | 4718.33 |
| 12.6 | 7.0 | 3488.46 |
| 12.8 | 6.9 | 11281.44 |
| 13.1 | 6.8 | 2684.87 |
| 13.5 | 6.5 | 1648.15 |
| 14.5 | 6.1 | 7367.71 |
| 15.8 | 5.6 | 35234.75 |
| 16.6 | 5.3 | 3504.83 |
| 18.1 | 4.9 | 41037.79 |
| 18.9 | 4.7 | 1062.55 |
| 19.2 | 4.6 | 6379.73 |
| 20.2 | 4.4 | 16176.19 |
| 20.3 | 4.4 | 17371.66 |
| 21.9 | 4.1 | 5995.19 |
| 22.2 | 4.0 | 6813.28 |
| 22.7 | 3.9 | 994.33 |
| 23.1 | 3.8 | 8769.11 |
| 23.9 | 3.7 | 16157.73 |
| 24.5 | 3.6 | 4099.43 |
| 24.9 | 3.6 | 927.84 |
| 25.5 | 3.5 | 5405.35 |
| 25.8 | 3.4 | 1470.89 |
| 26.1 | 3.4 | 2594.53 |
| 26.3 | 3.4 | 4109.63 |
| 27.1 | 3.3 | 6802.97 |
| 27.5 | 3.2 | 2786.18 |
| 28.6 | 3.1 | 2999.86 |
| 29.0 | 3.1 | 3257.84 |
| 29.9 | 3.0 | 1863.20 |
| 31.2 | 2.9 | 1010.52 |
| 31.9 | 2.8 | 1185.03 |
| 34.4 | 2.6 | 417.85 |
| 36.7 | 2.4 | 364.18 |
| 37.6 | 2.4 | 414.02 |
| 38.4 | 2.3 | 560.53 |

In some embodiments, polymorphic Form III has an XRPD pattern comprising peaks at angles 2-theta of 9.6±0.2, 10.9±0.2, 15.8±0.2, and 18.1±0.2 degrees. In some embodiments, polymorphic Form III has an XRPD pattern comprising peaks at angles 2-theta of 9.6±0.2, 10.9±0.2, 14.5±0.2, 15.8±0.2, and 18.1±0.2 degrees. In some embodiments, polymorphic Form III has an XRPD pattern comprising peaks at angles 2-theta of 9.6±0.2, 10.9±0.2, 14.5±0.2, 15.8±0.2, 18.1±0.2, and 20.2±0.2 degrees. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form III, can vary by about ±0.6 degrees, ±0.4 degrees, ±0.2 degrees, or ±0.1 degrees 2-theta.

In some embodiments, a mixture of polymorphic Forms I and III has a DSC graph substantially as shown in FIG. 28B.

In some embodiments, a mixture of polymorphic Forms I and III has a TGA graph substantially as shown in FIG. 28B.

### Form IV

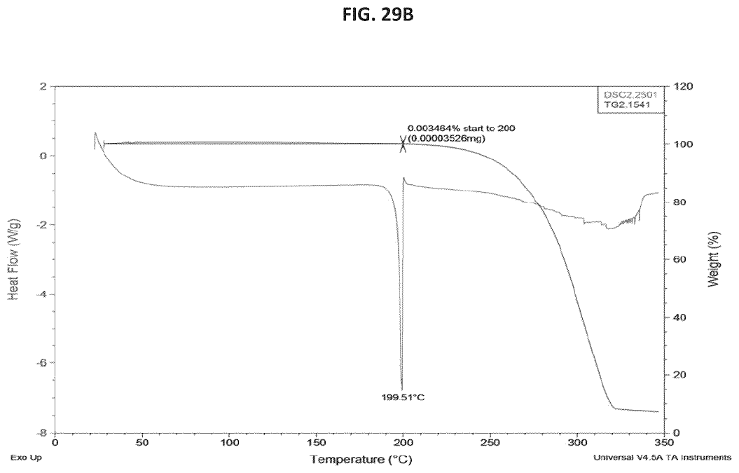

In some embodiments, the CK-274 is polymorphic Form IV of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form IV using XRPD are shown in Table P-4. In some embodiments, Form IV has an XRPD pattern substantially as shown in FIG. 29A.

TABLE P-4

| Angle (°2θ) | d value (Å) | Intensity (counts) |
|---|---|---|
| 3.7 | 23.6 | 20546.07 |
| 7.7 | 11.5 | 316.33 |
| 11.1 | 7.9 | 50282.89 |
| 12.4 | 7.1 | 9463.20 |
| 12.8 | 6.9 | 128952.37 |
| 13.5 | 6.6 | 73004.53 |
| 14.3 | 6.2 | 1107.15 |
| 15.5 | 5.7 | 1601.21 |
| 16.6 | 5.3 | 4393.72 |
| 17.9 | 4.9 | 18738.68 |
| 18.5 | 4.8 | 10243.00 |
| 18.6 | 4.8 | 5141.69 |
| 19.1 | 4.6 | 807.92 |
| 19.9 | 4.4 | 1686.97 |
| 20.9 | 4.2 | 14781.60 |
| 21.5 | 4.1 | 11233.06 |
| 21.6 | 4.1 | 8729.09 |
| 21.9 | 4.1 | 29321.27 |
| 22.3 | 4.0 | 16474.92 |
| 22.4 | 4.0 | 12987.42 |
| 22.8 | 3.9 | 35633.79 |
| 23.1 | 3.8 | 21970.49 |
| 23.5 | 3.8 | 24153.01 |
| 23.9 | 3.7 | 6592.59 |
| 24.4 | 3.6 | 42430.12 |
| 24.8 | 3.6 | 27600.62 |
| 25.0 | 3.6 | 18197.26 |
| 25.3 | 3.5 | 17487.34 |
| 25.8 | 3.4 | 20166.11 |
| 26.2 | 3.4 | 9283.12 |
| 27.1 | 3.3 | 1860.56 |
| 27.4 | 3.3 | 3534.99 |
| 28.0 | 3.2 | 2537.61 |
| 28.6 | 3.1 | 13746.55 |
| 29.0 | 3.1 | 1991.34 |
| 30.0 | 3.0 | 11236.64 |
| 30.5 | 2.9 | 5185.28 |
| 30.8 | 2.9 | 7657.41 |
| 31.0 | 2.9 | 2753.63 |
| 31.4 | 2.8 | 5359.89 |
| 33.8 | 2.6 | 1085.12 |
| 35.0 | 2.6 | 1833.47 |
| 35.7 | 2.5 | 1218.65 |
| 36.1 | 2.5 | 496.93 |
| 36.7 | 2.4 | 1373.07 |
| 37.9 | 2.4 | 2127.34 |
| 38.1 | 2.4 | 883.05 |
| 39.8 | 2.3 | 1200.46 |

In some embodiments, polymorphic Form IV has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 29A or as provided in Table P-4. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form IV, can vary by about ±0.6 degrees, ±0.4 degrees, ±0.2 degrees, or ±0.1 degrees 2-theta.

In some embodiments, polymorphic Form IV has an XRPD pattern comprising peaks at angles 2-theta of 3.7±0.2, 7.7±0.2, 11.1±0.2, 12.4±0.2, 12.8±0.2, 13.5±0.2, 14.3±0.2, 15.5±0.2, 16.6±0.2, 17.9±0.2, 18.5±0.2, 18.6±0.2, 19.1±0.2, 19.9±0.2, 20.9±0.2, 21.5±0.2, 21.6±0.2, 21.9±0.2, 22.3±0.2, 22.4±0.2, 22.8±0.2, 23.1±0.2, 23.5±0.2, 23.9±0.2, 24.4±0.2, 24.8±0.2, 25.0±0.2, 25.3±0.2, 25.8±0.2, 26.2±0.2, 27.1±0.2, 27.4±0.2, 28.0±0.2, 28.6±0.2, 29.0±0.2, 30.0±0.2, 30.5±0.2, 30.8±0.2, 31.0±0.2, 31.4±0.2, 33.8±0.2, 35.0±0.2, 35.7±0.2, 36.1±0.2, 36.7±0.2, 37.9±0.2, 38.1±0.2, 39.8±0.2 degrees. In some embodiments, polymorphic Form IV has an XRPD pattern comprising peaks at angles 2-theta of 3.7±0.2, 11.1±0.2, 12.8±0.2, 13.5±0.2, 21.9±0.2, 22.8±0.2, 23.1±0.2, 23.5±0.2, 24.4±0.2, and 24.8±0.2 degrees. In some embodiments, polymorphic Form IV has an XRPD pattern comprising peaks at angles 2-theta of 11.1±0.2, 12.8±0.2, 13.5±0.2, 22.8±0.2, and 24.4±0.2 degrees. It is to be understood that additional peaks in the XRPD pattern other than those shown in FIG. 29A or as provided in Table P-4 may be observed, for instance, due to the presence of impurities, solvent, or other polymorphs or amorphic forms present in the test sample.

In some embodiments, Form IV has a DSC graph substantially as shown in FIG. 29B. In some embodiments, Form IV is characterized as having an endotherm onset at about 200° C. as determined by DSC. In some embodiments, Form IV is characterized as having a melting endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.) as determined by DSC.

In some embodiments, Form IV has a TGA graph substantially as shown in FIG. 29B.

In some embodiments of Form IV, at least one, at least two, at least three, at least four, or all of the following (a)-(e) apply:
(a) Form IV has an XRPD pattern comprising peaks at angles 2-theta of 11.1±0.2, 12.8±0.2, 13.5±0.2, 22.8±0.2, and 24.4±0.2 degrees; an XRPD pattern comprising peaks at angles 2-theta of 3.7±0.2, 11.1±0.2, 12.8±0.2, 13.5±0.2, 21.9±0.2, 22.8±0.2, 23.1±0.2, 23.5±0.2, 24.4±0.2, and 24.8±0.2 degrees; or an XRPD pattern comprising peaks at angles 2-theta of 3.7±0.2, 7.7±0.2, 11.1±0.2, 12.4±0.2, 12.8±0.2, 13.5±0.2, 14.3±0.2, 15.5±0.2, 16.6±0.2, 17.9±0.2, 18.5±0.2, 18.6±0.2, 19.1±0.2, 19.9±0.2, 20.9±0.2, 21.5±0.2, 21.6±0.2, 21.9±0.2, 22.3±0.2, 22.4±0.2, 22.8±0.2, 23.1±0.2, 23.5±0.2, 23.9±0.2, 24.4±0.2, 24.8±0.2, 25.0±0.2, 25.3±0.2, 25.8±0.2, 26.2±0.2, 27.1±0.2, 27.4±0.2, 28.0±0.2, 28.6±0.2, 29.0±0.2, 30.0±0.2, 30.5±0.2, 30.8±0.2, 31.0±0.2, 31.4±0.2, 33.8±0.2, 35.0±0.2, 35.7±0.2, 36.1±0.2, 36.7±0.2, 37.9±0.2, 38.1±0.2, 39.8±0.2 degrees;
(b) Form IV has an XRPD pattern substantially as shown in FIG. 29A;
(c) Form IV has a DSC graph substantially as shown in FIG. 29B;
(d) Form IV is characterized as having a melting endotherm onset at about 200° C. as determined by DSC; and
(e) Form IV has a TGA graph substantially as shown in FIG. 29B.

### Form V

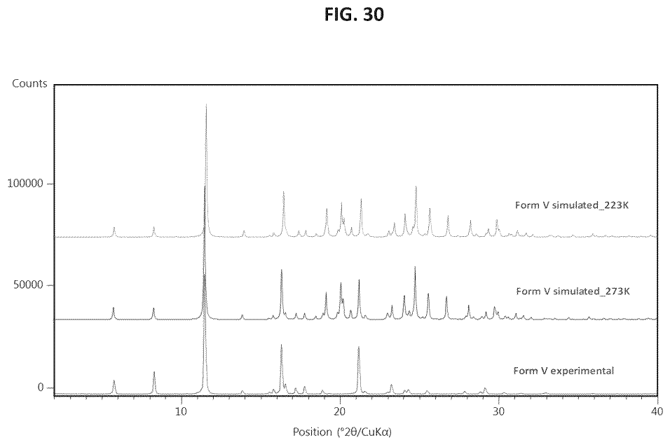

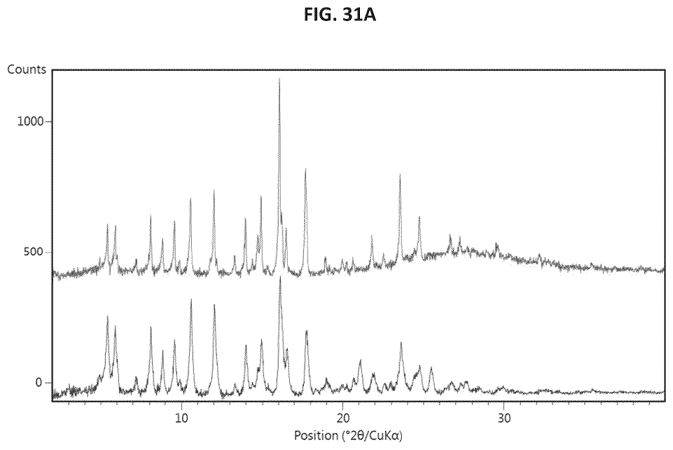

In some embodiments, the CK-274 is polymorphic Form V of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form V using XRPD are shown in Table P-5. In some embodiments, Form V has an XRPD pattern substantially as shown in FIG. 30.

TABLE P-5

| Angle (°2θ) | d value (Å) | Intensity (counts) |
|---|---|---|
| 5.7 | 15.4 | 920.22 |
| 8.3 | 10.7 | 902.83 |
| 11.5 | 7.7 | 9978.6 |
| 13.8 | 6.4 | 370.79 |
| 15.5 | 5.7 | 100.26 |
| 15.8 | 5.6 | 290.17 |
| 16.3 | 5.4 | 3772.89 |
| 16.6 | 5.4 | 397.64 |
| 17.2 | 5.1 | 500.92 |
| 17.8 | 5.0 | 473.82 |
| 18.5 | 4.8 | 237.44 |
| 18.9 | 4.7 | 388.87 |
| 19.1 | 4.6 | 2061.82 |
| 19.8 | 4.5 | 391.52 |
| 20.0 | 4.4 | 2661.48 |
| 20.2 | 4.4 | 1338.2 |
| 20.7 | 4.3 | 650.6 |
| 21.2 | 4.2 | 3071.64 |
| 21.6 | 4.1 | 295.42 |
| 23.0 | 3.9 | 446.39 |
| 23.1 | 3.9 | 165.78 |
| 23.3 | 3.8 | 1093.75 |
| 24.0 | 3.7 | 1807.48 |
| 24.2 | 3.7 | 176.77 |
| 24.3 | 3.7 | 472.09 |
| 24.6 | 3.6 | 216.92 |
| 24.7 | 3.6 | 3905.17 |
| 25.2 | 3.5 | 138.38 |
| 25.6 | 3.5 | 2004.45 |
| 26.7 | 3.3 | 1751.94 |
| 27.1 | 3.3 | 53.39 |
| 27.3 | 3.3 | 55.93 |
| 27.5 | 3.2 | 25.62 |
| 27.9 | 3.2 | 217.7 |
| 28.1 | 3.2 | 1093.17 |
| 28.4 | 3.1 | 215.32 |
| 28.9 | 3.1 | 222.86 |
| 29.2 | 3.1 | 615.11 |
| 29.7 | 3.0 | 856.96 |
| 29.8 | 3.0 | 361.62 |
| 29.9 | 3.0 | 544.6 |
| 30.4 | 2.9 | 248.45 |
| 30.6 | 2.9 | 181.85 |
| 31.1 | 2.9 | 481.57 |
| 31.3 | 2.9 | 57.68 |
| 31.5 | 2.8 | 312.95 |
| 32.0 | 2.8 | 174.91 |
| 32.9 | 2.7 | 101.41 |
| 33.0 | 2.7 | 61.9 |
| 33.2 | 2.7 | 71.42 |
| 33.5 | 2.7 | 99.57 |
| 34.4 | 2.6 | 162.66 |
| 34.6 | 2.6 | 42.98 |
| 34.9 | 2.6 | 15.79 |
| 35.3 | 2.5 | 33.86 |
| 35.7 | 2.5 | 241.09 |
| 36.0 | 2.5 | 67.72 |
| 36.2 | 2.5 | 17.68 |
| 36.5 | 2.5 | 12.09 |
| 36.6 | 2.5 | 100.18 |
| 37.0 | 2.4 | 39.38 |
| 37.1 | 2.4 | 63.03 |
| 37.5 | 2.4 | 49.61 |
| 37.8 | 2.4 | 59.59 |
| 37.9 | 2.4 | 68.66 |
| 38.3 | 2.4 | 18.63 |
| 38.4 | 2.3 | 28.07 |
| 38.7 | 2.3 | 21.55 |
| 38.8 | 2.3 | 52.54 |
| 39.3 | 2.3 | 31.1 |
| 39.4 | 2.3 | 123.61 |
| 39.6 | 2.3 | 51.44 |
| 39.9 | 2.3 | 74.51 |

In some embodiments, polymorphic Form V has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 30 or as provided in Table P-5. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form V, can vary by about ±0.6 degrees, ±0.4 degrees, ±0.2 degrees, or ±0.1 degrees 2-theta.

In some embodiments, polymorphic Form V has an XRPD pattern comprising peaks at angles 2-theta of 5.7±0.2, 8.3±0.2, 11.5±0.2, 13.8±0.2, 15.5±0.2, 15.8±0.2, 16.3±0.2, 16.6±0.2, 17.2±0.2, 17.8±0.2, 18.5±0.2, 18.9±0.2, 19.1±0.2, 19.8±0.2, 20.0±0.2, 20.2±0.2, 20.7±0.2, 21.2±0.2, 21.6±0.2, 23.0±0.2, 23.1±0.2, 23.3±0.2, 24.0±0.2, 24.2±0.2, 24.3±0.2, 24.6±0.2, 24.7±0.2, 25.2±0.2, 25.6±0.2, 26.7±0.2, 27.1±0.2, 27.3±0.2, 27.5±0.2, 27.9±0.2, 28.1±0.2, 28.4±0.2, 28.9±0.2, 29.2±0.2, 29.7±0.2, 29.8±0.2, 29.9±0.2, 30.4±0.2, 30.6±0.2, 31.1±0.2, 31.3±0.2, 31.5±0.2, 32.0±0.2, 32.9±0.2, 33.0±0.2, 33.2±0.2, 33.5±0.2, 34.4±0.2, 34.6±0.2, 34.9±0.2, 35.3±0.2, 35.7±0.2, 36.0±0.2, 36.2±0.2, 36.5±0.2, 36.6±0.2, 37.0±0.2, 37.1±0.2, 37.5±0.2, 37.8±0.2, 37.9±0.2, 38.3±0.2, 38.4±0.2, 38.7±0.2, 38.8±0.2, 39.3±0.2, 39.4±0.2, 39.6±0.2, and 39.9±0.2 degrees. In some embodiments, polymorphic Form V has an XRPD pattern comprising peaks at angles 2-theta of 5.7±0.2, 8.3±0.2, 11.5±0.2, 16.3±0.2, 17.2±0.2, 19.1±0.2, 20.0±0.2, 20.2±0.2, 20.7±0.2, 21.2±0.2, 23.3±0.2, 24.0±0.2, 24.7±0.2, 25.6±0.2, 26.7±0.2, 28.1±0.2, 29.2±0.2, 29.7±0.2, 29.9±0.2, and 31.1±0.2 degrees. In some embodiments, polymorphic Form V has an XRPD pattern comprising peaks at angles 2-theta of 11.5±0.2, 16.3±0.2, 19.1±0.2, 20.0±0.2, 20.2±0.2, 21.2±0.2, 24.0±0.2, 24.7±0.2, 25.6±0.2, and 26.7±0.2 degrees. In some embodiments, polymorphic Form V has an XRPD pattern comprising peaks at angles 2-theta of 11.5±0.2, 16.3±0.2, 20.0±0.2, 21.2±0.2, and 24.7±0.2 degrees. It is to be understood that additional peaks in the XRPD pattern other than those shown in FIG. 30 or as provided in Table P-5 may be observed, for instance, due to the presence of impurities, solvent, or other polymorphs or amorphic forms present in the test sample.

In some embodiments of Form V, at least one or both of the following (a)-(b) apply: (a) Form V has an XRPD pattern comprising peaks at angles 2-theta of 11.5±0.2, 16.3±0.2, 20.0±0.2, 21.2±0.2, and 24.7±0.2 degrees; an XRPD pattern comprising peaks at angles 2-theta of 11.5±0.2, 16.3±0.2, 19.1±0.2, 20.0±0.2, 20.2±0.2, 21.2±0.2, 24.0±0.2, 24.7±0.2, 25.6±0.2, and 26.7±0.2 degrees; or an XRPD pattern comprising peaks at angles 2-theta of 5.7±0.2, 8.3±0.2, 11.5±0.2, 16.3±0.2, 17.2±0.2, 19.1±0.2, 20.0±0.2, 20.2±0.2, 20.7±0.2, 21.2±0.2, 23.3±0.2, 24.0±0.2, 24.7±0.2, 25.6±0.2, 26.7±0.2, 28.1±0.2, 29.2±0.2, 29.7±0.2, 29.9±0.2, and 31.1±0.2 degrees; and
(b) Form V has an XRPD pattern substantially as shown in FIG. 30.

### Form VI

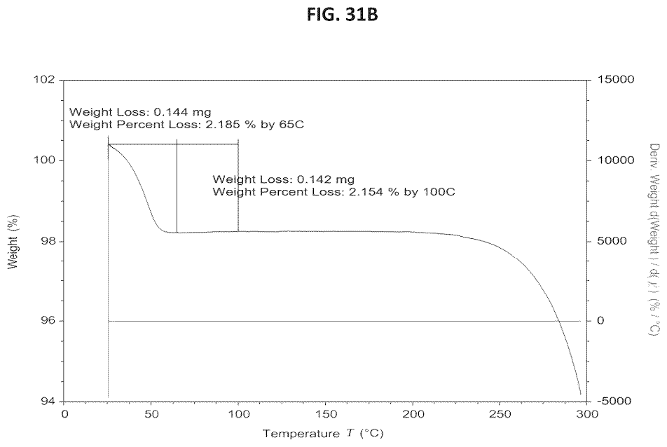

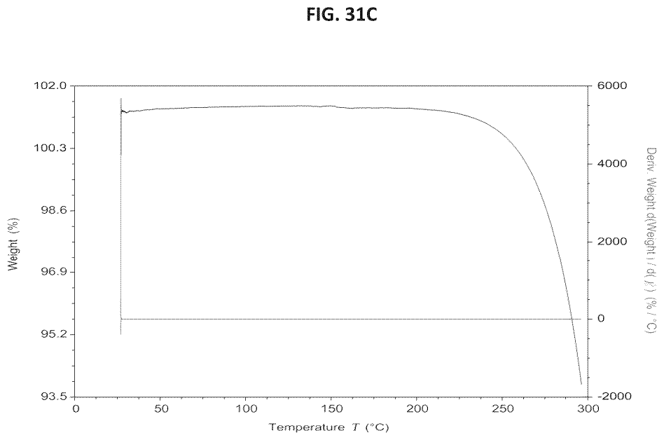

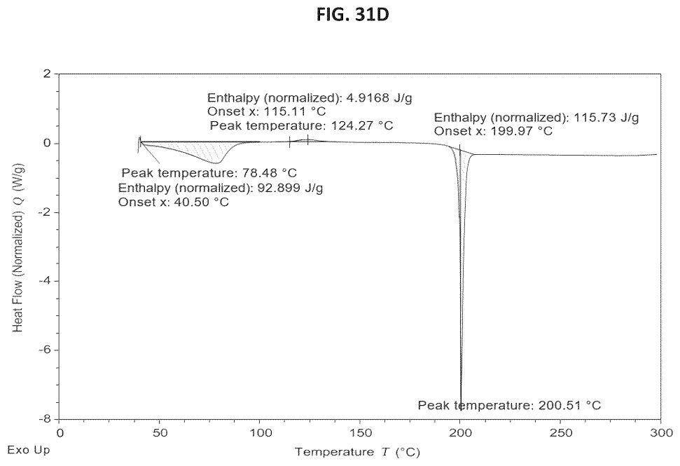

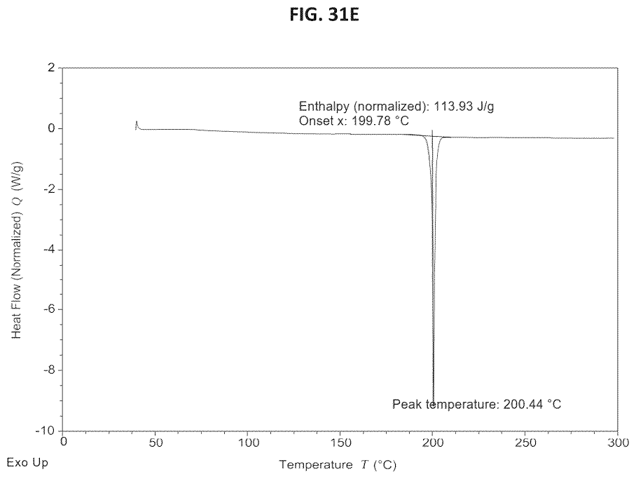

In some embodiments, the CK-274 is polymorphic Form VI of (R)—N-(5-(5-ethyl-1,2,4-oxadiazol-3-yl)-2,3-dihydro-1H-inden-1-yl)-1-methyl-1H-pyrazole-4-carboxamide. Angles 2-theta and relative peak intensities that may be observed for Form VI using XRPD are shown in Table P-6. In some embodiments, Form VI has an XRPD pattern substantially as shown in FIG. 31A.

TABLE P-6

| Angle (°2θ) | d value (Å) | Intensity (counts) |
|---|---|---|
| 3.0 | 29.4 | 54.91 |
| 5.0 | 17.7 | 127.50 |
| 5.4 | 16.3 | 453.97 |
| 5.9 | 14.9 | 587.10 |
| 7.2 | 12.3 | 125.69 |
| 8.1 | 10.9 | 567.03 |
| 8.9 | 10.0 | 345.88 |
| 9.6 | 9.2 | 432.70 |
| 9.9 | 8.9 | 79.69 |
| 10.6 | 8.3 | 788.69 |
| 12.1 | 7.3 | 797.99 |
| 13.3 | 6.6 | 159.40 |
| 14.0 | 6.3 | 476.49 |
| 14.4 | 6.1 | 77.78 |
| 14.7 | 6.0 | 220.91 |
| 15.0 | 5.9 | 593.87 |
| 15.4 | 5.8 | 46.97 |
| 16.1 | 5.5 | 1350.47 |
| 16.5 | 5.4 | 388.67 |
| 17.8 | 5.0 | 805.27 |
| 18.9 | 4.7 | 55.59 |
| 19.0 | 4.7 | 100.12 |
| 19.2 | 4.6 | 53.05 |
| 19.6 | 4.5 | 24.26 |
| 20.0 | 4.4 | 98.96 |
| 20.3 | 4.4 | 55.64 |
| 20.7 | 4.3 | 145.05 |
| 21.1 | 4.2 | 226.01 |
| 21.9 | 4.1 | 246.53 |
| 22.6 | 3.9 | 115.35 |
| 22.9 | 3.9 | 78.78 |
| 23.6 | 3.8 | 399.26 |
| 23.6 | 3.8 | 373.80 |
| 23.8 | 3.7 | 58.05 |
| 24.4 | 3.6 | 146.88 |
| 24.8 | 3.6 | 348.39 |
| 25.5 | 3.5 | 226.63 |
| 26.4 | 3.4 | 42.15 |
| 26.7 | 3.3 | 137.53 |
| 27.3 | 3.3 | 112.85 |
| 27.6 | 3.2 | 120.57 |
| 28.2 | 3.2 | 55.34 |
| 28.5 | 3.1 | 60.37 |
| 29.0 | 3.1 | 26.44 |
| 29.6 | 3.0 | 70.01 |
| 29.9 | 3.0 | 60.95 |
| 30.4 | 2.9 | 49.00 |
| 30.9 | 2.9 | 14.91 |
| 31.6 | 2.8 | 12.97 |
| 32.2 | 2.8 | 38.90 |
| 32.6 | 2.7 | 40.95 |
| 33.1 | 2.7 | 16.26 |
| 33.3 | 2.7 | 16.56 |
| 34.5 | 2.6 | 10.35 |
| 35.0 | 2.6 | 16.35 |
| 35.5 | 2.5 | 36.10 |
| 38.5 | 2.3 | 20.00 |

In some embodiments, polymorphic Form VI has an XRPD pattern displaying at least two, at least three, at least four, at least five, at least six, at least seven, at least eight, at least nine, or at least ten of the peaks at angles 2-theta with the greatest intensity in the XRPD pattern substantially as shown in FIG. 31A or as provided in Table P-6. It should be understood that relative intensities can vary depending on a number of factors, including sample preparation, mounting, and the instrument and analytical procedure and settings used to obtain the spectrum. Relative peak intensities and peak assignments can vary within experimental error. In some embodiments, peak assignments listed herein, including for polymorphic Form VI, can vary by about ±0.6 degrees, ±0.4 degrees, ±0.2 degrees, or ±0.1 degrees 2-theta.

In some embodiments, polymorphic Form VI has an XRPD pattern comprising peaks at angles 2-theta of 3.0±0.2, 5.0±0.2, 5.4±0.2, 5.9±0.2, 7.2±0.2, 8.1±0.2, 8.9±0.2, 9.6±0.2, 9.9±0.2, 10.6±0.2, 12.1±0.2, 13.3±0.2, 14.0±0.2, 14.4±0.2, 14.7±0.2, 15.0±0.2, 15.4±0.2, 16.1±0.2, 16.5±0.2, 17.8±0.2, 18.9±0.2, 19.0±0.2, 19.2±0.2, 19.6±0.2, 20.0±0.2, 20.3±0.2, 20.7±0.2, 21.1±0.2, 21.9±0.2, 22.6±0.2, 22.9±0.2, 23.6±0.2, 23.8±0.2, 24.4±0.2, 24.8±0.2, 25.5±0.2, 26.4±0.2, 26.7±0.2, 27.3±0.2, 27.6±0.2, 28.2±0.2, 28.5±0.2, 29.0±0.2, 29.6±0.2, 29.9±0.2, 30.4±0.2, 30.9±0.2, 31.6±0.2, 32.2±0.2, 32.6±0.2, 33.1±0.2, 33.3±0.2, 34.5±0.2, 35.0±0.2, 35.5±0.2, and 38.5±0.2 degrees. In some embodiments, polymorphic Form VI has an XRPD pattern comprising peaks at angles 2-theta of 5.4±0.2, 5.9±0.2, 8.1±0.2, 9.6±0.2, 10.6±0.2, 12.1±0.2, 14.0±0.2, 15.0±0.2, 16.1±0.2, and 17.8±0.2 degrees. In some embodiments, polymorphic Form VI has an XRPD pattern comprising peaks at angles 2-theta of 10.6±0.2, 12.1±0.2, 15.0±0.2, 16.1±0.2, and 17.8±0.2 degrees. It is to be understood that additional peaks in the XRPD pattern other than those shown in FIG. 31A or as provided in Table P-6 may be observed, for instance, due to the presence of impurities, solvent, or other polymorphs or amorphic forms present in the test sample.

In some embodiments, Form VI has a TGA graph substantially as shown in FIG. 31B or a TGA graph substantially as shown in FIG. 31C. In some embodiments, Form VI exhibits a weight loss of about 2%±0.5% between 25° C. and 200° C. as determined by TGA.

In some embodiments, Form VI has a DSC graph substantially as shown in FIG. 31D or a DSC graph substantially as shown in FIG. 31E. In some embodiments, Form VI is characterized as having a melting endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.) as determined by DSC. In some embodiments, Form VI is characterized as having an endotherm onset at about 200±2° C. (e.g., 200±1.9° C., 200±1.8° C., 200±1.7° C., 200±1.6° C., 200±1.5° C., 200±1.4° C., 200±1.3° C., 200±1.2° C., 200±1, 200±0.9° C., 200±0.8° C., 200±0.7° C., 200±0.6° C., 200±0.5° C., 200±0.4° C., 200±0.3° C., 200±0.2° C., or 200±0.1° C.), an exotherm onset at about 115±2° C. (e.g., 115±1.9° C., 115±1.8° C., 115±1.7° C., 115±1.6° C., 115±1.5° C., 115±1.4° C., 115±1.3° C., 115±1.2° C., 115±1, 115±0.9° C., 115±0.8° C., 115±0.7° C., 115±0.6° C., 115±0.5° C., 115±0.4° C., 115±0.3° C., 115±0.2° C., or 115±0.1° C.), or an endotherm onset at about 41±2° C. (e.g., 41±1.9° C., 41±1.8° C., 41±1.7° C., 41±1.6° C., 41±1.5° C., 41±1.4° C., 41±1.3° C., 41±1.2° C., 41±1, 41±0.9° C., 41±0.8° C., 41±0.7° C., 41±0.6° C., 41±0.5° C., 41±0.4° C., 41±0.3° C., 41±0.2° C., or 41±0.1° C.), or any combinations thereof.

In some embodiments of Form VI, at least one, at least two, at least three, at least four, at least five, at least six or all of the following (a)-(g) apply:
(a) Form VI has an XRPD pattern comprising peaks at angles 2-theta of 10.6±0.2, 12.1±0.2, 15.0±0.2, 16.1±0.2, and 17.8±0.2 degrees; an XRPD pattern comprising peaks at angles 2-theta of 5.4±0.2, 5.9±0.2, 8.1±0.2, 9.6±0.2, 10.6±0.2, 12.1±0.2, 14.0±0.2, 15.0±0.2, 16.1±0.2, and 17.8±0.2 degrees; or an XRPD pattern comprising peaks at angles 2-theta of 3.0±0.2, 5.0±0.2, 5.4±0.2, 5.9±0.2, 7.2±0.2, 8.1±0.2, 8.9±0.2, 9.6±0.2, 9.9±0.2, 10.6±0.2, 12.1±0.2, 13.3±0.2, 14.0±0.2, 14.4±0.2, 14.7±0.2, 15.0±0.2, 15.4±0.2, 16.1±0.2, 16.5±0.2, 17.8±0.2, 18.9±0.2, 19.0±0.2, 19.2±0.2, 19.6±0.2, 20.0±0.2, 20.3±0.2, 20.7±0.2, 21.1±0.2, 21.9±0.2, 22.6±0.2, 22.9±0.2, 23.6±0.2, 23.8±0.2, 24.4±0.2, 24.8±0.2, 25.5±0.2, 26.4±0.2, 26.7±0.2, 27.3±0.2, 27.6±0.2, 28.2±0.2, 28.5±0.2, 29.0±0.2, 29.6±0.2, 29.9±0.2, 30.4±0.2, 30.9±0.2, 31.6±0.2, 32.2±0.2, 32.6±0.2, 33.1±0.2, 33.3±0.2, 34.5±0.2, 35.0±0.2, 35.5±0.2, and 38.5±0.2 degrees;
(b) Form VI has an XRPD pattern substantially as shown in FIG. 31A;
(c) Form VI has a TGA graph substantially as shown in FIG. 31B or FIG. 31C.
(d) Form VI has a weight loss of about 2%±0.5% between 25° C. and 200° C. as determined by TGA;
(e) Form VI has a DSC graph substantially as shown in FIG. 31D or FIG. 31E; and
(f) Form IV is characterized as having a melting endotherm onset at about 200° C. as determined by DSC; and
(g) Form VI is characterized as having an endotherm onset at about 200° C., an exotherm onset at about 115° C., or an endotherm onset at about 41° C., or any combination thereof, as determined by DSC.

### Kits

Also provided are articles of manufacture and kits containing any of the compounds or pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing a compound or composition described herein and instructions for use. The kits may contain instructions for use in the treatment of a heart disease in an individual or subject in need thereof. A kit may additionally contain any materials or equipment that may be used in the administration of the compound or composition, such as vials, syringes, or IV bags. A kit may also contain sterile packaging.

In some embodiments, the present disclosure provides methods for manufacturing a medicament comprising CK-274, comprising:
manufacturing a first collection of tablets the amounts of CK-274 (which may exist in various forms) in which are about, or are about half of, a first daily dose;
manufacturing a second collection of tablets the amounts of CK-274 (which may exist in various forms) in which are about, or are about half of, a second daily dose;
optionally manufacturing a third collection of tablets the amounts of CK-274 (which may exist in various forms) in which are about, or are about half of, a third daily dose; and
optionally manufacturing a fourth collection of tablets the amounts of CK-274 (which may exist in various forms) in which are about, or are about half of, a fourth daily dose.

In some embodiments, a method comprises manufacturing a third collection of tablets the amounts of CK-274 (which may exist in various forms) in which are about, or are about half of, a third daily dose. In some embodiments, a method comprises manufacturing a fourth collection of tablets the amounts of CK-274 (which may exist in various forms) in which are about, or are about half of, a fourth daily dose.

In some embodiments, the present disclosure provides methods for manufacturing medicament comprising CK-274, comprising:
manufacturing a first collection of tablets the amounts of CK-274 (which may exist in various forms) in which are about a first daily dose;
manufacturing a second collection of tablets the amounts of CK-274 (which may exist in various forms) in which are about a second daily dose;
optionally manufacturing a third collection of tablets the amounts of CK-274 (which may exist in various forms) in which are about a third daily dose; and
optionally manufacturing a fourth collection of tablets the amounts of CK-274 (which may exist in various forms) in which are about a fourth daily dose.

In some embodiments, a method comprises manufacturing a third collection of tablets the amounts of CK-274 (which may exist in various forms) in which are about a third daily dose. In some embodiments, a method comprises manufacturing a fourth collection of tablets the amounts of CK-274 (which may exist in various forms) in which are about a fourth daily dose.

Various first, second, third and fourth daily doses are described herein. In some embodiments, a daily dose is about 5 mg of CK-274. In some embodiments, a first daily dose is about 5 mg of CK-274. In some embodiments, a daily dose is about 10 mg of CK-274. In some embodiments, a daily dose is about 15 mg of CK-274. In some embodiments, a daily dose is about 20 mg of CK-274. In some embodiments, tablets with at least two different daily doses are manufactured. In some embodiments, tablets the amounts of CK-274 (which may exist in various forms) in which are about 15 mg are manufactured. In some embodiments, tablets the amounts of CK-274 (which may exist in various forms) in which are about 2.5 mg are manufactured. In some embodiments, tablets the amounts of CK-274 (which may exist in various forms) in which are about 7.5 mg are manufactured. Those skilled in the art will appreciate that in addition to CK-274, tablets may comprise various other components as described herein, e.g., filler, binder, disintegrant, surfactant, lubricant, etc. In some embodiments, tablets are film-coated.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1

This first-in-human study of aficamten (also referred to as CK-274) was undertaken to evaluate its safety, pharmacokinetic, and pharmacodynamic profile, including effects of food or a CYP2D6 poor metabolizer (CYP2D6-PM) phenotype. Aficamten, a selective cardiac myosin inhibitor, reduced measures of left ventricular contractility preclinically in vitro and in vivo, and therefore, may have therapeutic potential for the management of hypertrophic cardiomyopathy. This phase 1, double-blind, randomized, placebo-controlled study enrolled healthy adults, aged 18 to 55 years, to receive single-ascending doses or multiple-ascending doses (14 or 17 days) of aficamten or placebo. In addition to standard safety and pharmacokinetic assessments, pharmacodynamic effects were assessed by echocardiography. The study enrolled 102 participants (57 in single-dose, 24 in multiple-dose, 9 in CYP2D6-PM, and 12 in food-effect cohorts). At single doses of aficamten ≤50 mg and multiple doses ≤10 mg, adverse events were generally mild and no more frequent than with placebo. In single-ascending dose cohorts, plasma concentrations of aficamten increased in a dose-proportional manner; the half-life of aficamten was 75 to 85 h. Neither food nor CYP2D6-PM phenotype had a clinically meaningful impact on pharmacokinetics. With a single 50-mg dose, mean left ventricular ejection fraction (LVEF) decreased from baseline by 5.5% (p=0.0001). With multiple doses, a mean reduction in LVEF of 5.0% was observed after 14 days of 10 mg of aficamten once daily. Aficamten appears safe and well tolerated at doses evaluated. A pharmacodynamic effect on LVEF was demonstrated, providing support for further clinical investigations of aficamten.

### Methods

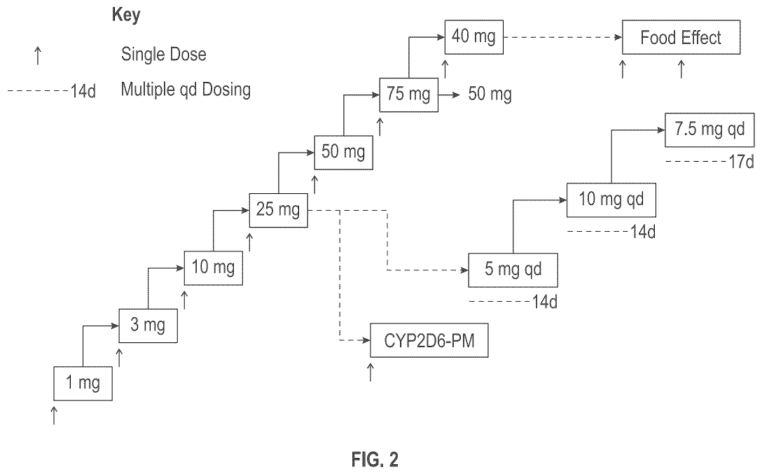

Study Overview and Ethics. The study used a randomized, placebo-controlled, single-ascending dose (SAD) and multiple-ascending dose (MAD) design (FIG. 2). The study was not designed to identify a maximum tolerated dose, but rather to identify a pharmacologically active dose range, defined as giving an absolute reduction in left ventricular ejection fraction (LVEF) from baseline in the range of 5% to 15% (such as, a baseline LVEF value of 70% reduced to between 55% to 65%). Dose escalation was to be stopped when this range was achieved, or when a non-tolerated dose was identified, if earlier.

Participants and Treatments. To be eligible for this study, participants were to be healthy adults aged 18 to 55 years, with body mass index of 18.0 to 32.0 kg/m², normal electrocardiogram (ECG) and clinical laboratory values, or only minor abnormalities that were deemed not clinically significant. Participants also had to have normal cardiac structure and function, with LVEF ≥60% for the first 4 SAD cohorts, ≥65% for subsequent SAD cohorts, all MAD cohorts, and the food-effect cohort, and ≥55% for the CYP2D6-PM cohort. Before the study, participants were not allowed to use any prescription medication within 14 days, over-the-counter medication within 7 days (except acetaminophen), or tobacco or nicotine within 3 months; in addition, they were not permitted to consume alcohol, caffeine, or grapefruit within 48 h before study check-in.

A randomization schedule was centrally generated for each cohort and treatment period. In all cohorts, aficamten or matching placebo was administered in granule form in a capsule with ˜240 ml of water. Study drug was administered following an overnight fast, except during the fed period in the food-effect cohort.

Single-ascending dose (SAD) cohorts. The SAD portion of the study used a randomized, double-blind, placebo-controlled, sequential, escalating-dose design, in which participants received single-ascending oral doses of the study drug. Seven cohorts were dosed in sequence (FIG. 2). Of the 8 participants within each cohort, the first 2 were randomly assigned (1:1) to aficamten or placebo and followed for a minimum of 2 days before the remainder of the group was dosed. The remaining 6 participants were then randomly assigned (5:1) to receive either oral single doses of aficamten (1, 3, 10, 25, 40, 50, or 75 mg) or placebo.

The initial dose of aficamten was selected using criteria from the United States Food and Drug Administration guidance based on prior animal studies and employing a safety margin of ≥10-fold. Dose escalation would stop when results identified a pharmacologically active dose range that reduced LVEF by 5% to 15% or a non-tolerated dose, whichever occurred first.

Recommendations regarding dose escalation in the SAD cohorts—and in the MAD cohorts described below—were made by the treating investigator (who was blinded to treatment group) and endorsed or not by the Dose Level Review Committee (DLRC), who were unblinded. Decisions were made when ≥6 participants had been treated and followed for ≥3 days, including collection of clinical, laboratory, ECG, and telemetry data, and echocardiograms suitable for assessing LV function around the time of maximum plasma drug concentration (Cₘₐₓ). Criteria for escalation included that no more than 2 participants in a dose group developed an LVEF <50% and no individual developed an LVEF <45%. Dose escalation criteria were as follows: (1) no individual had sustained a cardiac serious adverse event related to the study drug; (2) no 2 individuals had experienced similar, non-cardiac serious adverse events in the same organ system that seemed to be related to the study drug; (3) no 2 individuals treated with aficamten experienced a decrease in left ventricular ejection fraction (LVEF) ≥15% in comparison with the last pre-dose value (determined by the Dose Level Review Committee [DLRC]); (4) no individual developed an LVEF <45% (unless determined not to be related to the study drug by the DLRC and the treating investigator); and (5) both the treating investigator and DLRC approved the escalation and next level dose based on their clinical judgment.

Multiple-ascending dose (MAD) cohorts. The MAD cohorts also used a randomized, double-blind, placebo-controlled, sequential design. Enrollment in the MAD cohorts began when the SAD cohorts identified a single oral dose that was well tolerated and associated with an observed PD effect. Each of the 3 MAD cohorts included 8 participants, randomized (6:2) to aficamten or placebo. Participants received oral doses of the study drug once daily for 14 days (in the cohorts comparing 5 or 10 mg of aficamten vs. placebo) or 17 days (for the cohort comparing 7.5 mg of aficamten vs. placebo).

CYP2D6 poor metabolizer cohort. A separate cohort was enrolled to evaluate the potential impact of CYP2D6 genetic variants on the PK properties of aficamten. The CYP2D6 gene encodes the cytochrome P450 2D6 enzyme, described as the most extensively characterized polymorphic drug-metabolizing enzyme, and prior in vitro studies had implicated CYP2D6 as a potential metabolizing enzyme of aficamten.

CYP2D6 genotypes were determined at screening for all study participants; those identified as CYP2D6-PMs were excluded from the SAD and MAD cohorts but were invited to participate in the CYP2D6-PM cohort. The first individual in the CYP2D6-PM cohort was dosed after the SAD 25-mg cohort (FIG. 2). Each participant received a single dose of aficamten (10 mg) or placebo. Nine participants were randomized (7:2) with a sentinel dosing group consisting of the first 2 participants treated.

Food-effect cohort. To assess the effect of food on the PK of aficamten, a separate cohort was enrolled after completion of the last SAD cohort, with enrollment of 8 to 12 participants planned. In an open-label, 2-way crossover design, participants were to receive 2 single doses of 10 mg of aficamten, separated by ≥14 days. Participants were randomized in a 1:1 ratio to 1 of 2 sequences: fasted/fed or fed/fasted. In the fasted period, aficamten was administered after an overnight fast; in the fed period, aficamten was administered 30 minutes after the start of a high-fat breakfast.

### Assessment

Safety and tolerability. Safety was assessed by the incidence of adverse events (AEs) and by incidence of reduced LVEF. Treatment-emergent AEs (TEAE) were defined as AEs that began or increased after study drug administration. All AEs were coded using the Medical Dictionary for Regulatory Activities version 21.1 and graded using the National Cancer Institute Common Terminology Criteria for AEs (version 4.03) 5-point severity scale. Each AE was judged as either related or unrelated to the study drug by the treating investigator. Clinical laboratory tests were obtained at regular intervals in all cohorts.

For safety monitoring, participants in all cohorts had periodic echocardiograms, which were assessed by a cardiologist. In the SAD and MAD cohorts, echocardiograms were also reviewed by an echocardiography core laboratory for PD assessments, as described below. In addition, participants in all cohorts were monitored with continuous 12-lead ECG recording using Holter monitors. For safety monitoring, a single 12-lead ECG was extracted at screening, pre-dose, and periodically throughout follow-up, and interpreted by the investigator. In the SAD, MAD, and CYP2D6-PM cohorts, cardiodynamic ECGs (triplicate 10-second, 12-lead ECG recordings) were obtained prior to the corresponding PK blood and ECG intervals quantitated by qualified readers.

Pharmacokinetic analyses. For all study groups, blood samples for PK assessment were obtained pre-dose, up to 12 times daily on day 1, and then at regular intervals throughout the study. Blood samples were collected according to the following schedule: SAD Cohorts: Day 1: pre-dose and 0.25, 0.5, 1, 1.5, 2, 2.5, 3, 4, 6, 8, 12, 16, 24, 36, 48, 72, 96, and 216 h post dose. MAD Cohorts (14-day Dosing): Day 1: pre-dose and 0.25, 0.5, 1, 1.5, 2, 2.5, 3, 4, 6, 8, and 12 h post dose; Days 2, 4, 5, 6, and 9: pre-dose (corresponding to trough samples following dosing on days 1, 3, 4, 5, and 8) and 1.5 h post dose; Days 3, 7, 8, 10, 11, 12, and 13: pre-dose (corresponding to trough samples following dosing on days 2, 6, 7, 9, 10, 11, and 12); Day 14: pre-dose and 0.25, 0.5, 1.5, 2, 2.5, 3, 4, 6, 8, 12, 16, 24, 36, 48, 72, and 168 h post dose. MAD Cohort (17-Day Dosing): Day 1: pre-dose and 0.25, 0.5, 1, 1.5, 2, 2.5, 3, 4, 6, 8, and 12 h post dose; Days 2, 4, 5, 6, and 9: pre-dose (corresponding to trough samples following dosing on days 1, 3, 4, 5, and 8) and 1.5 h post dose; Days 3, 7, 8, 10, 11, 12, 13, 14, 15, and 16: pre-dose (corresponding to trough samples following dosing on days 2, 6, 7, 9, 10, 11, and 12); Day 17: pre-dose and 0.25, 0.5, 1.5, 2, 2.5, 3, 5, 7, 9, 12, 24, 36, 48, 72, and 168 h post dose. CYPD6-PM Cohort: Day 1: pre-dose and 0.25, 0.5, 1, 1.5, 2, 2.5, 3, 4, 6, 8, 12, 16, 24, 36, 48, 72, 96, 216, 312, and 552 h post dose. Food-Effect Cohort: Day 1: pre-dose and 0.25, 0.5, 1, 1.5, 2, 2.5, 3, 4, 6, 8, 12, 16, 24, 36, 48, 72, 96, 144, and 216 h post dose. Standard non-compartmental methods were used to calculate PK parameters using Phoenix® WinNonlin® Version 7.0; actual sample collection times were utilized.

Plasma concentrations of aficamten were measured utilizing high performance liquid chromatography-tandem mass spectrometry methods validated for accuracy, precision, linearity, sensitivity, and specificity at Celerion (Lincoln, Nebraska). The analytical range (the lower to upper limits of quantitation) for aficamten was 1.00 to 500 ng/ml.

Echocardiography. For PD assessments of LVEF, echocardiograms for the SAD and MAD cohorts were interpreted by the echocardiography core laboratory and used for all data analysis and dose level review decisions while immediate local interpretation of the echocardiograms was performed for safety monitoring. In the SAD cohorts receiving 1, 3, or 10 mg of aficamten, echocardiograms were obtained on day −1, pre-dose on day 1, and at 1.5, 4, and 24 h post dose. For SAD cohorts receiving 25, 40, 50, or 75 mg of aficamten, echocardiograms were obtained on day −1, pre-dose on day 1, and at 1.5, 6, and 24 h post dose. Echocardiograms on day 3 (48 h after dosing) were obtained only if the 24 h LVEF had not returned to near or above baseline, as determined by the investigator. In the MAD cohorts, echocardiograms were obtained on day −1, pre-dose on day 1, and at 1.5 h post dose on days 2, 4, and 9, and at 1.5, 24, and 72 h post dose on day 14 (for the 5-mg and 10-mg cohorts) or on day 17 (for the 7.5-mg cohort). Echocardiograms were obtained 3 days after the last dose (on day 17 or 20) only if the participant's prior LVEF was not near or above baseline, as determined by the investigator.

Statistical Analysis. The sample size chosen for this study was based upon precedent set by other first-in-human PK studies of similar nature and was not based on power calculations. All participants who received ≥1 dose of the study drug (aficamten or placebo) were included in safety analyses. All participants who received ≥1 dose of the study drug and had ≥1 evaluable PK plasma profile were included in the PK analysis set.

The PK analyses were intended to assess single-dose kinetics, multiple-dose (steady state) kinetics, the influence of the CYP2D6 phenotype on absorption and elimination of aficamten, and the influence of food on the absorption and elimination of aficamten. For the SAD cohort, dose proportionality of aficamten was evaluated using a power model on day 1.

For the MAD cohort, dose proportionality was evaluated using a power model on day 1 and day 14 or 17. Several considerations were taken into account when assessing dose proportionality of the drug, such as results derived from the power model statistical analysis (e.g., the slope estimate and width of the 2-sided 95% confidence intervals [CIs]), qualitative assessment specific to the PK of the drug, and clinical relevance. For the SAD cohort, the parameters used to assess dose proportionality were area under the plasma drug concentration-time curve (AUC) from time 0 to the time of the last measurable concentration (AUCₗₐₛₜ), AUC from time 0 extrapolated to infinity (AUC_{inf}), AUC from time 0 to 24 h (AUC₂₄), and maximum plasma concentration (Cₘₐₓ). For the MAD cohort, the parameters were AUC₂₄ and Cₘₐₓ on day 1, plus AUC to the end of the dosing period (AUCₜₐᵤ) and Cₘₐₓ on day 14 or 17. The statistical linear relationship between the In-transformed PK parameters and the In-transformed dose was verified by including the quadratic (ln Dose)² and cubic (ln Dose)³ effects. The statistical linear relationship was established if the quadratic and cubic effects were not statistically significant, using a 5% level of significance, or if the effects were statistically significant, but of such small magnitude that they were not clinically relevant. The dose-proportionality analysis was performed using SAS® PROC MIXED. If a statistical linear relationship was shown and if the 2-sided 95% CIs around the slope estimate parameters included the value of 1 for dose-dependent parameters, then dose proportionality was established.

In the MAD cohort, a steady-state analysis for aficamten was performed on the In-transformed plasma trough concentration (C_{trough}) values using Helmert contrasts. An analysis of variance (ANOVA) model was conducted separately for each dose level; day was included as the fixed effect. Helmert contrasts were developed such that each time-point was compared with the mean of the subsequent time-points. Steady state was established at the time-point where no statistical difference (alpha=5%, 2-sided) was observed with the subsequent time-points.

All participants who received ≥1 dose of study drug and had ≥1 pre-dose and ≥1 post-dose echocardiographic measurement were included in the PD analysis set. Descriptive analyses included absolute reduction in LVEF relative to baseline and categorical LVEF responses (proportions of participants with reduction in LVEF from baseline of ≥5%, ≥10%, and ≥15% and proportions of participants with LVEF <50% and ≤45%). Descriptive statistics of the echocardiographic parameters were generated using SAS® Version 9.3 or higher.

Dose-response analysis was performed using analysis of covariance (ANCOVA) to identify the least-squares mean difference (aficamten minus placebo). To analyze the impact of drug dose on echocardiographic parameters in the SAD and MAD cohorts, an inferential analysis was conducted on the PD analysis set using linear mixed models for repeated-measures analysis of covariance (ANCOVA). The ANCOVA used baseline value as a covariate, included treatment, time-point, and time-point-by-treatment interaction as fixed effects, and change from baseline as the dependent variable. The unstructured variance-covariance structure was used, and the model accounted for the time-point repeated measures. The ANCOVA analysis was conducted separately for each study part and for each PD parameter. The least-squares means, the difference in least squares means (active minus placebo), and the associated 2-sided 95% CIs were presented for each comparison.

Analyses of concentration ‘bin’ and exposure-response were also performed using ANCOVA. SAS® PROC MIXED was used for all comparative analyses. An additional inferential analysis was performed in the SAD and MAD cohorts, to evaluate the relationship between concentrations of aficamten and LVEF for participants in the PK/PD analysis set. A concentration bin ANCOVA was conducted using linear mixed models for repeated-measures analyses with concentration bin group as a fixed effect, baseline PD parameters as a covariate, change from baseline as the dependent variable, and a random intercept to adjust for the repeated measures. The unstructured variance-covariance structure was used. Plasma concentrations of aficamten were paired with coincident PD parameters. The ANCOVA compared the change in PD parameters between each bin versus the pooled placebo group.

The least-squares means, the difference in least squares means (bin group minus placebo), and the associated 2-sided 95% CIs were presented for each comparison. The ANCOVA analysis was conducted separately for each study part. For all time-points at which both PK data and PD measures were available, the time-points were pooled for the analysis. In each part of the study, aficamten concentrations with time-matched PD data were pooled and sorted in increasing order. From least to greatest, the data were then divided into 5 groups of observations (‘bins’), each consisting of 20% of the data points. Each bin was treated as a separate group. Concentration bins consisted of a placebo group and 5 bin groups based on the pool of concentrations from all time-points on aficamten treatment.

The above analysis was then repeated using concentration as a continuous variable to estimate the exposure-response trend. Both a random intercept effect and a random concentration effect were added to the ANCOVA. The estimate of the concentration slope, with corresponding 2-sided 95% CIs, and the ANCOVA analyses were presented for each study part.

For all time-points at which both PK data and PD measures were available, the time points were pooled for the analysis. A nominal significance level of 5% was used for statistical comparisons, without adjustment for multiplicity.

### Results

Study Population. A total of 102 participants were enrolled: 57 in the SAD cohorts, 24 in the MAD cohorts, 9 in the CYP2D6-PM cohort, and 12 in the food-effect cohort. All participants completed the study. Mean age ranged between 32 and 40 years across cohorts, and the majority of participants were male (Table 1).

TABLE 1
Baseline Characteristics

| | SAD | | MAD | | CYP2D6-PM | | Food-Effect | |
|---|---|---|---|---|---|---|---|---|
| | (n = 57) | | (n = 24) | | (n = 9) | | (n = 12) | |
| Age, years | 39.6 | (18-55) | 40.4 | (28-54) | 32.0 | (20-47) | 39.6 | (25-51) |
| Male | 41 | (72) | 18 | (75) | 9 | (100) | 9 | (75) |
| White race | 52 | (91) | 17 | (71) | 9 | (100) | 11 | (92) |
| Hispanic or Latino ethnicity | 42 | (74) | 13 | (54) | 4 | (44) | 8 | (67) |

| | | | | |
|---|---|---|---|---|
| Weight, kg | 79.4 ± 10.3 | 77.0 ± 9.1 | 84.8 ± 12.0 | 76.4 ± 11.1 |
| Height, cm | 169.0 ± 9.4 | 168.9 ± 10.0 | 177.8 ± 7.8 | 168.8 ± 9.2 |
| BMI, kg/m² | 27.8 ± 2.7 | 27.0 ± 2.3 | 26.8 ± 3.3 | 26.8 ± 3.0 |
| LVEF, % | 65.8 ± 2.4 | 67.5 ± 1.2 | 61.0 ± 2.6 | 67.2 ± 1.2 |

Values are mean (range), n (%), or mean ± SD. BMI = body mass index; LVEF = left ventricular ejection fraction; MAD = multiple-ascending dose; CYP2D6-PM = cytochrome P450 2D6 poor metabolizer; SAD = single-ascending dose; SD = standard deviation.

For the SAD cohorts, there were no safety concerns that prohibited dose escalation between 1 mg and 25 mg. With the next planned dose (50 mg), 1 participant had a post-dose LVEF <50% (46.2%); however, this did not meet the dose escalation stopping rules and the 75-mg cohort was initiated. The sentinel participant in the 75-mg cohort had LVEF <45% post dose; consequently, no further participants were dosed at 75 mg. As a result, the 50-mg group was expanded, and an additional 5 participants were dosed in this cohort. Following the expansion, 1 participant in the 50-mg dose group experienced LVEF <45%, which was also a decrease of >15%. Therefore, no further participants were dosed at ≥50 mg. The DLRC determined the appropriate dose for the final single-dose cohort was 40 mg.

Following results from the 1-mg to 25-mg SAD cohorts, the first MAD cohort was initiated at 5 mg of aficamten once daily for 14 days. There were no safety concerns, and the next cohort was initiated at 10 mg once daily for 14 days. In this cohort, 2 participants met the stopping criteria based on echocardiography results. The DLRC decided the next treatment level should be 7.5 mg, to better characterize the PK at steady state; thus, the dosing period was extended from 14 to 17 days to ensure PK had reached steady state by the last day of dosing.

Safety and Tolerability. There were no serious AEs, and no participants discontinued the study due to AEs. The TEAEs that were observed were generally mild (grade 1) and no more frequent with aficamten than with placebo for both single-dose and multiple-dose administration (Table 2 and Table 3). Overall, the most common TEAE was headache in both the SAD and MAD cohorts (Table 2 and Table 3).

TABLE 2
Treatment-Emergent Adverse Events in the SAD Cohorts

| | Pooled | Aficamten SAD Cohort, mg | | | | | | | |
|---|---|---|---|---|---|---|---|---|---|
| | Placebo | 1 | 3 | 10 | 25 | 40 | 50 | 75 | Total |
| | (n = 15) | (n = 6) | (n = 6) | (n = 6) | (n = 6) | (n = 6) | (n = 11) | (n = 1) | (N = 57) |
| Participants with any TEAE | 4 (27) | 1 (17) | — | 1 (17) | 5 (83) | 1 (17) | 4 (36) | 1 (100) | 17 (30) |
| Most common TEAEs | | | | | | | | | |
| Headache | 1 (7) | — | — | 1 (17) | 2 (33) | — | 1 (9) | — | 5 (9) |
| Ejection fraction decreased | | — | — | — | — | 1 (17) | 1 (9) | 1 (100) | 3 (5) |
| Abdominal tenderness | | — | — | — | 1 (17) | — | 1 (9) | — | 2 (4) |
| Chest pain | | — | — | — | — | — | 1 (9)* | 1 (100)^{†} | 2 (4) |
| Dyspepsia | | — | — | — | 1 (17) | — | 1 (9) | — | 2 (4) |
| Flatulence | | — | — | — | 1 (17) | — | 1 (9) | — | 2 (4) |
| Nausea | | — | — | — | 2 (33) | — | — | — | 2 (4) |
| Upper abdominal pain | | — | — | — | 2 (33) | — | — | — | 2 (4) |
| Upper respiratory tract infection | 2 (13) | — | — | — | — | — | — | — | 2 (4) |

Values are n (%). Most common TEAEs based on preferred terms, reported in ≥2 participants in the total cohort.
*‘Bubbling sensation to the left chest’ consistent with a gastrointestinal association rather than a cardiac association.
^{†}Onset approximately 60 h post dose.
SAD = single-ascending dose;
TEAE = treatment-emergent adverse event.

TABLE 3
Treatment-Emergent Adverse Events in the MAD Cohorts

| | | Aficamten MAD Cohorts | | | |
|---|---|---|---|---|---|
| | Pooled | 5 mg qd × | 7.5 mg qd × | 10 mg qd × | |
| | Placebo | 14 Days | 17 Days | 14 Days | Total |
| | (n = 6) | (n = 6) | (n = 6) | (n = 6) | (N = 24) |
| Participants with any TEAE | 1 (17) | 1 (17) | — | 2 (33) | 4 (17) |
| TEAEs | | | | | |
| Headache | 1 (17) | — | — | 1 (17) | 2 (8) |
| Chapped lips | — | 1 (17) | — | — | 1 (4) |
| Cough | — | 1 (17) | — | — | 1 (4) |
| Feeling hot | 1 (17) | — | — | — | 1 (4) |
| Nausea | 1 (17) | — | — | — | 1 (4) |
| Salivary hypersecretion | — | 1 (17) | — | — | 1 (4) |
| Upper respiratory tract infection | — | — | — | 1 (17) | 1 (4) |
| Vomiting | 1 (17) | — | — | — | 1 (4) |

Values are n (%).
TEAEs based on preferred terms, reported in ≥1 participant in the total cohort.
MAD = multiple-ascending dose;
qd = once daily;
TEAE = treatment-emergent adverse event.

Echocardiogram-related AEs of decreased ejection fraction <45% based on the study echocardiogram expert assessment were reported in 3 participants: 1 each in the SAD 40-mg, 50-mg, and 75-mg cohorts (Table 4). All were grade 1, and all resolved at the next echocardiogram assessment (within 2.5 to 4.6 h). The single participant who received 75 mg of aficamten had LVEF of 34.6% at 1.5 h post dose, which was a reduction in LVEF of 31.5% and led to concluding escalation of doses in the SAD portion of the study as discussed above. At the following assessment 2.5 h later, LVEF had returned to 51.9%. No AEs of decreased ejection fraction <45% were reported in the MAD, CYP2D6-PM, or food-effect cohorts.

In all cohorts, mean safety ECG parameters at the assessed time-points were within normal limits. No clinically notable changes from baseline were observed among any of the parameters. The QT interval corrected for heart rate using Fridericia's formula (QTcF) did not exceed 450 ms either at baseline or at any assessment during the dosing interval, with the exception of 2 individuals whose baseline QTcF was ≥440 ms and whose QTcF values increased by 3 and 13 ms, respectively. In all cohorts, there were no increases in QTcF interval >30 ms, with the exception of 1 participant in the SAD placebo group whose QTcF interval increased by 33 ms on day 5 (427 ms vs. a baseline value of 394 ms). In the cardiodynamic assessments, categorical analysis of ECG parameters revealed no cardiac safety concerns, and there was no evidence of a positive QT effect following single or multiple doses of aficamten.

All vital signs were within normal limits at the post-dose time-points. No clinically significant serum chemistry, hematology, or urinalysis findings were observed during the study.

TABLE 4
Echocardiogram-Related AEs of Decreased Ejection Fraction <45%

| Cohort, | | | | LVEF | |
|---|---|---|---|---|---|
| Participant | Dose | Grade | Onset* | Deemed AE^{†} | Outcome^{‡} |
| SAD, | 40 mg | 1 (mild) | 1.4 h | 42.2% | Resolved 4.6 h later |
| 42-year-old | | | | | (6 h after drug dose), |
| white female | | | | | with LVEF recorded at |
| | | | | | 58.4% |
| SAD, | 50 mg | 1 (mild) | 1.5 h | 42.1% | Resolved 2.5 h later |
| 46-year-old | | | | | (4 h after drug dose), |
| white male | | | | | with LVEF recorded at |
| | | | | | 50.2% |
| SAD, | 75 mg | 1 (mild) | 1.5 h | 34.6% | Resolved 2.5 h later |
| 36-year-old | | | | | (4 h after drug dose), |
| white/Hispanic | | | | | with LVEF recorded at |
| male | | | | | 51.9% |

*Time after drug dose.
^{†}LVEF values for safety assessments were determined by the study cardiologist.
^{‡}In the SAD cohorts, post-dose echocardiograms were performed at 1.5, 4, 6, 24, and 48 h post dose.
AE = adverse event; LVEF = left ventricular ejection fraction; SAD = single-ascending dose.

### Pharmacokinetics

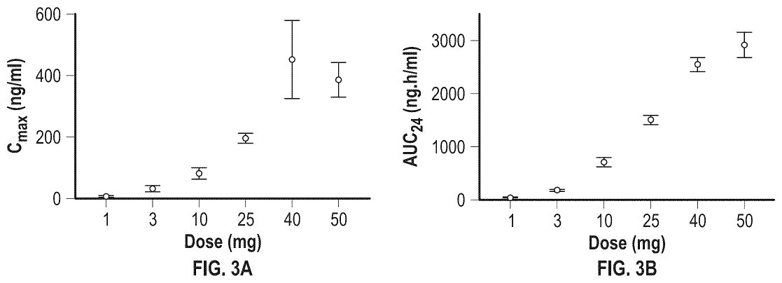

Single-dose kinetics. The plasma aficamten profiles were generally well characterized for all dose levels with the exception of the lowest dose of 1 mg (due to concentrations close to the lower limit of quantitation) and the highest dose of 75 mg, which was administered to only 1 participant, as discussed above. Over the dose range of 1 mg to 50 mg, mean maximal plasma concentrations and exposure increased in a dose-proportional manner, as demonstrated by the rise in Cₘₐₓ and area under the plasma concentration-time curve from time 0 to 24 h (AUC₂₄) with increasing doses (FIG. 3A-3B and Table 5). Mean clearance and volume of distribution were similar across the doses. Median time to maximum observed concentration occurred between 0.5 and 2.8 h, with a maximum time of 4.0 h across all participants. Mean half-life ranged from 75 to 85 h.

TABLE 5
Summary of Plasma Aficamten Pharmacokinetics Following Single Oral Dose Administration

| | Aficamten SAD Cohort, mg | | | | | | |
|---|---|---|---|---|---|---|---|
| | 1 | 3 | 10 | 25 | 40 | 50 | 75 |
| Cₘₐₓ | 5.8 (38.4) | 26.1 (82.4) | 70.5 (69.0) | 192.6 (23.3) | 383.2 (67.9) | 359.4 (38.9) | 1,220 (—) |
| (ng/ml) | n = 6 | n = 6 | n = 6 | n = 6 | n = 6 | n = 11 | n = 1 |
| Tₘₐₓ (h) | 1.0 (0.5, 4.0) | 1.3 (0.5, 2.6) | 2.8 (0.5, 3.0) | 1.8 (0.5, 4.0) | 1.0 (0.5, 1.5) | 1.0 (0.5, 2.5) | 0.5 (0.5, 0.5) |
| | n = 6 | n = 6 | n = 6 | n = 6 | n = 6 | n = 11 | n = 1 |
| AUC₂₄ | 50 (20) | 174 (27) | 679 (35) | 1,493 (15) | 2,532 (13) | 2,833 (26) | 4,556 (—) |
| (ng · h/ml) | n = 6 | n = 6 | n = 6 | n = 6 | n = 6 | n = 11 | n = 1 |
| AUC_{inf} | — | 823 (61) | 3,434 (24) | 7,113 (15) | 11,860 (20) | 13,740 (35) | |
| (ng · h/ml) | — | n = 5 | n = 4 | n = 6 | n = 5 | n = 9 | |
| Vz/F (L) | — | 392.7 ± 122.2 | 361.7 ± 108.0 | 405.3 ± 56.6 | 381.7 ± 66.9 | 431.3 ± 122.9 | |
| | — | n = 5 | n = 4 | n = 6 | n = 5 | n = 9 | |
| t_{1/2} (h) | — | 75.1 ± 21.8 | 84.7 ± 17.7 | 79.8 ± 10.0 | 79.2 ± 18.5 | 80.4 ± 14.9 | |
| | — | n = 5 | n = 4 | n = 6 | n = 4 | n = 9 | |
| CL/F (L/h) | — | 4.2 ± 3.1 | 3.0 ± 0.7 | 3.6 ± 0.5 | 3.4 ± 0.6 | 3.8 ± 1.1 | |
| | — | n = 5 | n = 4 | n = 6 | n = 5 | n = 9 | |

Cₘₐₓ and AUC values are presented as geometric mean and geometric CV %, Tₘₐₓ as median (range), and all other parameters as arithmetic mean ± standard deviation.
AUC₂₄ = area under the plasma drug concentration-time curve from time 0 to 24 h;
AUC_{inf} = area under the plasma drug concentration-time curve from time 0 extrapolated to infinity;
CL/F = apparent total body clearance;
Cₘₐₓ = maximum plasma concentration;
CV % = percent coefficient of variation;
t_{1/2} = half-life;
Tₘₐₓ = time to maximum plasma concentration;
Vz/F = apparent volume of distribution.

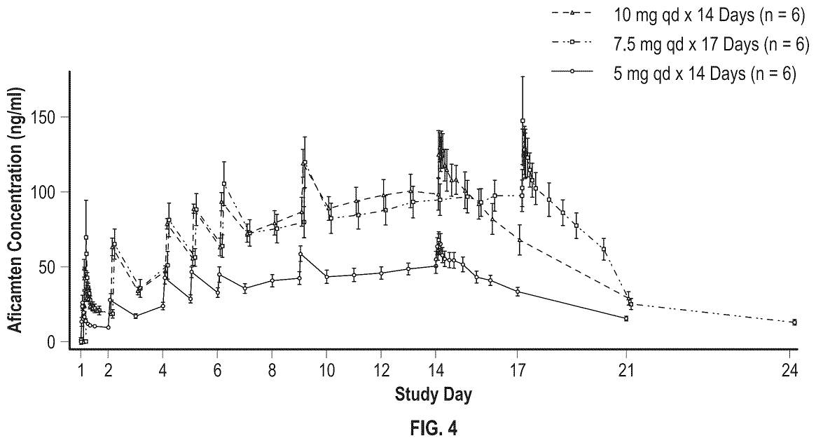

Multiple-dose kinetics. With once-daily dosing, mean plasma concentrations increased between the 5-mg dose and the 2 higher doses (7.5 mg and 10 mg); however, by day 2 there was little difference between mean concentrations of the 7.5-mg and 10-mg doses (FIG. 4). Plasma PK parameters are displayed in Table 6. By the end of the treatment period (day 14 or 17), the mean plasma concentration was between 2 and 2.5 times that of day 1. Terminal elimination half-life estimates were consistent across doses, ranging between 77 and 86 h. Clearance was similar for the 5-mg and 10-mg doses, and the accumulation ratio was similar for the 3 doses. Consistent with the observed terminal elimination half-life estimates, steady state was achieved after 10 to 12 days (FIG. 4).

TABLE 6
Summary of Plasma Aficamten Pharmacokinetics
Following Multiple Oral Dose Administration

| Multiple-Ascending Dose (MAD) Cohorts | | |
|---|---|---|
| 5 mg qd × 14 Days | 7.5 mg qd × 17 Days | 10 mg qd × 14 Days |
| (n = 6) | (n = 6) | (n = 6) |

Day 1

| | | | | | | |
|---|---|---|---|---|---|---|
| Cₘₐₓ (ng/ml) | 28.0 | (30.1) | 69.3 | (66.5) | 54.4 | (25.1) |
| Tₘₐₓ (h) | 1.3 | (0.5, 2.5) | 0.8 | (0.5, 1.5) | 1.0 | (1.0, 2.5) |
| AUC₂₄ (ng · h/ml) | 278 | (21) | 551 | (33) | 547 | (11) |

Day 14/17

| | | | | | | |
|---|---|---|---|---|---|---|
| C_{max, ss} (ng/ml) | 69.0 | (23) | 147.6 | (39.5) | 141.2 | (19.7) |
| T_{max, ss} (h) | 2.8 | (1.5, 4.0) | 1.0 | (0.5, 5.0) | 2.5 | (0.5, 3.0) |
| AUCₜₐᵤ (ng · h/ml) | 1320 | (23) | 2518 | (26) | 2632 | (23) |

| | | | |
|---|---|---|---|
| t_{1/2} (h) | 86.4 ± 11.9 | 76.9 ± 14.5 | 79.8 ± 14.1 |
| CLₛₛ/F (h) | 3.9 ± 0.9 | 3.1 ± 0.7 | 3.9 ± 0.8 |
| RA, _{AUC} | 4.8 ± 0.2 | 4.6 ± 0.7 | 4.9 ± 0.9 |

AUC₂₄, Cₘₐₓ, AUCₜₐᵤ, and C_{max, ss} values are presented as geometric mean and geometric CV %, Tₘₐₓ and T_{max, ss} values as median (range), and all other parameters as arithmetic mean ± standard deviation. AUC₂₄ = area under the plasma drug concentration-time curve from time 0 to 24 h; AUCₜₐᵤ = AUC to the end of the dosing period; CLₛₛ/F = apparent total body clearance after oral administration (at steady state); Cₘₐₓ = maximum plasma concentration; C_{max, ss} = maximum plasma concentration at steady state; CV % = percent coefficient of variation; RA, _{AUC} = accumulation ratio calculated from AUCₜₐᵤ at steady and following a single dose; t_{1/2} = half-life; Tₘₐₓ = time to maximum plasma concentration; T_{max, ss} = time to reach maximum plasma concentration following drug administration at steady state.

CYP2D6 poor metabolizer cohort. In CYP2D6-PMs, mean half-life was prolonged to 110 h, compared with 85 h in extensive metabolizers (i.e., the 10-mg SAD cohort); however, no increase in AUC was observed in this group, with geometric mean AUC₂₄ of 495 ng·h/ml (geometric percent coefficient of variation [CV %] 19) (Table 7), compared with 679 ng·h/ml (geometric CV % 35) in extensive metabolizers (Table 5). The CYP2D6-PMs did not appear to have reduced clearance that resulted in a clinically meaningful difference in exposures.

TABLE 7
Summary of Plasma Aficamten Pharmacokinetics
in the CYP2D6 Poor Metabolizer Cohort

| | CYP2D6 Poor Metabolizers | |
|---|---|---|
| PK Parameter | (n = 7) | |
| Cₘₐₓ (ng/ml) | 57.3 | (55.0) |
| Tₘₐₓ (h) | 1.0 | (0.5, 4.0) |
| AUC₂₄ (ng · h/ml) | 495 | (19) |
| AUC_{inf} (ng · h/ml) | 2966 | (46) |

| | |
|---|---|
| t_{1/2} (h) | 110.2 ± 47.3 |
| CL/F (L/h) | 3.6 ± 1.4 |
| Vz/F (L) | 506.0 ± 84.4 |

Aficamten dosage was 10 mg. AUC and Cₘₐₓ values are presented as geometric mean and geometric CV %, Tₘₐₓ values as median (range), and all other parameters as arithmetic mean ± standard deviation. AUC₂₄ = area under the plasma drug concentration-time curve from time 0 to 24 h; AUC_{inf} = AUC from time 0 extrapolated to infinity; CL/F = apparent total body clearance; Cₘₐₓ = maximum plasma concentration; CV % = percent coefficient of variation; PK = pharmacokinetic; t_{1/2} = half-life; Tₘₐₓ = time to maximum plasma concentration; Vz/F = apparent volume of distribution.

Effect of food. The PK parameters of aficamten in the food-effect cohort are displayed in Table 8. When taken with food, the Cₘax of aficamten increased by approximately 30% and the time to maximum observed concentration was reduced (1.5 vs. 2.3 h). However, food had little effect on AUC, with geometric mean AUC₂₄ (geometric CV %) in the fasted state of 601 (33) ng·h/ml versus 631 (25) in the fed state.

TABLE 8
Summary of Plasma Aficamten Pharmacokinetics
in the Food-Effect Cohort

| | Fasted | | Fed | |
|---|---|---|---|---|
| PK Parameter | (n = 10)* | | (n = 12)^{†} | |
| Cₘₐₓ (ng/ml) | 50.6 | (47.2) | 65.0 | (69.5) |
| Tₘₐₓ (h) | 2.3 | (0.6, 6.0) | 1.5 | (0.5, 6.0) |
| AUC₂₄ (ng · h/ml) | 601 | (33) | 631 | (25) |
| AUC_{inf} (ng · h/ml) | 3674 | (24) | 3413 | (32) |

| | | |
|---|---|---|
| t_{1/2} (h) | 84.4 ± 16.4 | 78.0 ± 8.1 |
| CL/F (L/h) | 2.8 ± 0.6 | 3.1 ± 0.9 |
| Vz/F (L) | 345.7 ± 133.7 | 339.7 ± 100.4 |

Aficamten dosage was 10 mg in each cohort.
*n = 4 for AUC_{inf}, t_{1/2}, CL/F, and Vz/F.
^{†}n = 7 for AUC_{inf}, t_{1/2}, CL/F, and Vz/F. AUC and Cₘₐₓ values are presented as geometric mean and geometric CV %,
Tₘₐₓ as median (range), and all other parameters as arithmetic mean ± standard deviation.
AUC₂₄ = area under the plasma drug concentration-time curve from 0 to 24 h;
AUC_{inf} = AUC from time 0 extrapolated to infinity;
CL/F = apparent total body clearance;
Cₘₐₓ = maximum plasma concentration;
CV % = percent coefficient of variation;
t_{1/2} = half-life;
Tₘₐₓ = time to maximum plasma concentration;
Vz/F = apparent volume of distribution.

### Pharmacodynamics

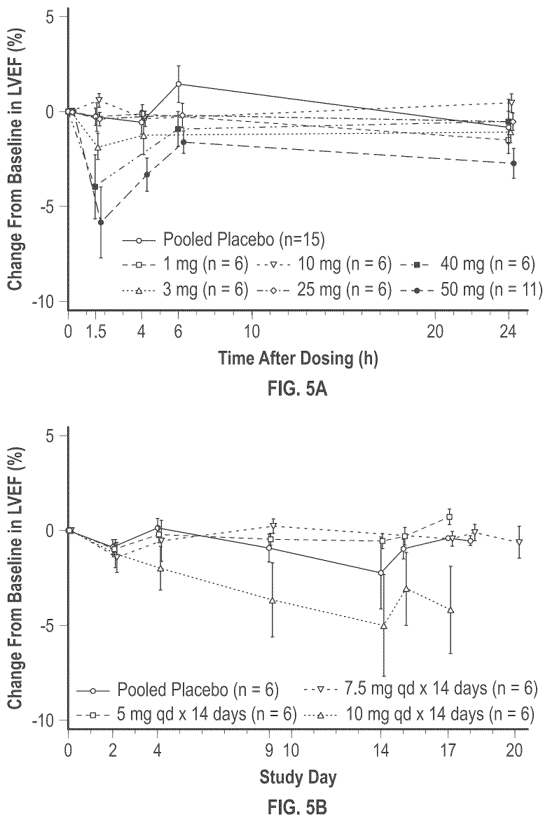

Left ventricular ejection fraction. At baseline, mean LVEF ranged from 61.0% to 67.5% across cohorts (Table 1). In the SAD cohorts, mean decreases in LVEF were observed in the groups receiving the highest doses of aficamten (FIG. 5A). Maximum mean reduction from baseline was seen in the 50-mg cohort at 1.5 h post dose (least-squares mean difference 5.5%, p=0.0001). LVESV and LVEDV were statistically significantly increased by 8.1 and 6.6 mL, respectively (Table 9). Other echocardiographic parameters such as stroke volume, cardiac output, cardiac time intervals, and measures reflective of diastolic function did not significantly change (Table 9). The single participant who received 75 mg of aficamten exhibited reduction in LVEF of 31.5% at 1.5 h post dose, which resolved 2.5 h after onset but led to concluding escalation of doses in the SAD portion of the study, as discussed above. In the MAD cohorts, a clear decrease in LVEF emerged as dosing continued in the 10-mg cohort (FIG. 5B). The largest mean maximum percent reduction from baseline, of 5.0%, was seen in the 10-mg cohort at 1.5 h post dose on day 14 (FIG. 5B). The placebo-corrected reduction of 3.2% (least-squares mean difference) did not reach statistical significance (p=0.21), likely due to the lack of statistical power in this small group comparison.

TABLE 9
Echocardiographic Parameters

| | Placebo (n = 15) | | | | | | 50 mg (n = 11) | | | | | | p value |
|---|---|---|---|---|---|---|---|---|---|---|---|---|---|
| | Baseline | | 1.5 h | | CFB | | Baseline | | 1.5 h | | CFB | | CFB v |
| | Mean | SD | Mean | SD | Mean | SD | Mean | SD | Mean | SD | Mean | SD | CFB |
| Heart rate (bpm) | 61.6 | 5.6 | 61.4 | 7.6 | −0.2 | 5.6 | 61.7 | 11.5 | 62.3 | 10.6 | 0.5 | 5.8 | NS |
| LVEF (%) | 66 | 2 | 65.8 | 2 | −0.3 | 1.8 | 66.3 | 3.8 | 60.5 | 7.3 | −5.8 | 6.1 | 0.003 |
| LV ESV (mL) | 32 | 5.5 | 31.1 | 5.5 | −0.9 | 3.6 | 31.6 | 6.8 | 39.7 | 10.4 | 8.1 | 8.4 | 0.001 |
| LV EDV (mL) | 93.6 | 15.2 | 90.6 | 14.4 | −3 | 8.1 | 93.2 | 13.2 | 99.8 | 13.4 | 6.6 | 10.6 | 0.016 |
| LV SV (mL) | 56.7 | 6.3 | 56.1 | 9.7 | −0.6 | 6.8 | 58.9 | 8 | 52.6 | 10.5 | −6.2 | 7.6 | NS |
| LV CO (mL) | 3470 | 299 | 3450 | 655 | −21.4 | 538 | 3580 | 597 | 3240 | 681 | −346 | 498 | NS |
| IVCT (msec) | 46.9 | 2.4 | 46.9 | 6.4 | 0.4 | 5.9 | 42.3 | 1.6 | 42.6 | 1.8 | 0.4 | 2.9 | NS |
| IVRT (msec) | 72.1 | 10.6 | 73.9 | 7 | 1.9 | 11.3 | 68.7 | 3 | 69.3 | 3.3 | 0.5 | 3.8 | NS |
| LV ET (msec) | 332 | 32.2 | 324 | 26.3 | −6.9 | 12.5 | 344 | 35.1 | 328 | 34.1 | 1.9 | 12.8 | NS |
| LAV (mL) | 36 | 8.4 | 35.3 | 9.3 | −1 | 7.6 | 35.7 | 9.9 | 35.7 | 7.2 | 0 | 4.7 | NS |
| E (cm/sec) | 73.6 | 6.7 | 68.2 | 8.2 | −5.3 | 5.7 | 66.7 | 14.7 | 64 | 14.7 | −2.7 | 4.5 | NS |
| A (cm/sec) | 49.4 | 11.4 | 47.7 | 10.3 | −1.7 | 5.6 | 52 | 12.8 | 54.5 | 17.5 | 2.5 | 6.6 | NS |
| e′ lateral (cm/sec) | 12.7 | 2.5 | 12.3 | 2.7 | −0.7 | 1.5 | 11.5 | 3.1 | 10.2 | 2.4 | −1.3 | 1.6 | NS |
| E/A ratio | 1.6 | 0.4 | 1.5 | 0.4 | −0.1 | 0.2 | 1.3 | 0.1 | 1.2 | 0.2 | −0.1 | 0.1 | NS |
| E/e′ ratio | 6 | 1.2 | 5.8 | 1.3 | −0.1 | 0.7 | 6.3 | 2.8 | 6.8 | 3.3 | 0.5 | 1.5 | NS |

The table compares the placebo data of echocardiographic parameters at baseline and 1.5 h (the time of the echocardiogram closest to peak of plasma concentrations of aficamten) to those at 50 mg of aficamten (the highest well tolerated single dose).
A = peak A wave velocity,
bpm = beat per minute,
CFB = change from baseline,
CO = cardiac output,
E = peak E wave velocity,
EDV = end-systolic volume,
ESV = end-systolic volume,
ET = ejection time,
e′ lateral = tissue doppler velocity of the lateral wall,
IVCT = isovolumic contraction time,
IVRT = isovolumic relaxation time,
LAV = left atrial volume,
LVEF = left ventricular ejection fraction,
NS = not significant (p > 0.05),
SD = Standard deviation,
SV = stroke volume

Categorical LVEF responses. In the SAD cohorts, absolute reductions in LVEF of ≥5% from baseline were observed in 1 of 15 participants in the placebo cohort (7%), 1 of 6 (17%) in the 3-mg cohort, 2 of 6 (33%) in the 40-mg cohort, 7 of 11 (64%) in the 50-mg cohort, and 1 of 1 (100%) in the 75-mg cohort, while no participants in the 1-, 10-, or 25-mg cohorts had a reduction ≥5%. Absolute reductions in LVEF of ≥10% occurred in 1 of 6 participants (17%) in the 40-mg cohort, 2 of 11 (18%) in the 50-mg cohort, and 1 (100%) in the 75-mg cohort. Reduction in LVEF to <50% was observed for 2 of 11 participants (18%) in the 50-mg cohort (48.2% and 45.5% per core laboratory assessment), and 1 of 1 (100%) in the 75-mg cohort. Only the participant in the 75-mg cohort experienced LVEF <45%.

In the MAD cohort, absolute reduction in LVEF of ≥5% from baseline was observed in 4 participants: 1 of 6 participants (17%) receiving placebo, 1 of 6 (17%) receiving 7.5 mg of aficamten once daily, and 2 of 6 (33%) receiving 10 mg of aficamten once daily. Of these, reductions were ≥10% in the 2 participants in the 10-mg cohort. Reduction in LVEF to <50% was not observed in any of the MAD cohorts per core laboratory assessment.

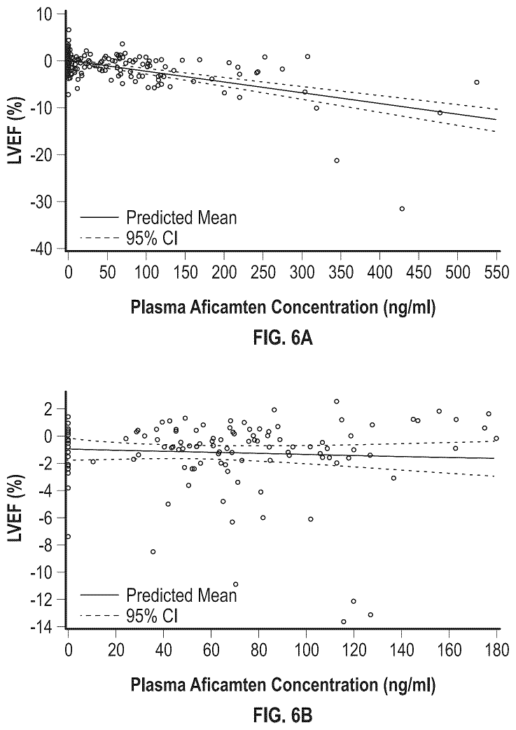

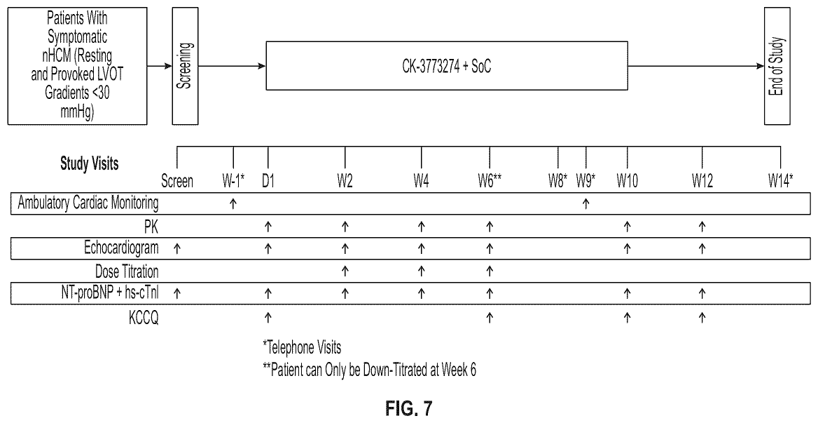

Relationship of Plasma Concentration to Change in LVEF. The PK/PD relationship for aficamten is illustrated by plotting the plasma concentration of aficamten versus change in LVEF for the SAD and MAD cohorts (FIG. 6A and FIG. 6B). In the SAD cohort, as plasma concentration of aficamten increased, there was a trend toward a decrease in LVEF. The relationship of LVEF to the plasma concentration of aficamten was statistically significant, both in the bin concentration analysis for the highest plasma concentration bin (122 to 524 ng/ml, p<0.0001) and in the concentration-slope analysis (p=0.0027). In the MAD cohorts, the relationship of LVEF to plasma aficamten did not reach statistical significance in the bin concentration analysis or the linear regression analysis, likely due to the more limited range of plasma concentrations explored and the small group sizes.

### Discussion

This phase 1, first-in-human study has established the doses (up to 50 mg as a single oral dose or up to 10 mg following multiple doses) at which aficamten was both physiologically effective at reducing LVEF and was well tolerated in healthy participants, identifying pharmacologically active doses that will serve as starting doses for a study in patients with HCM. In addition, single oral doses of 10 mg were well tolerated among individuals with the CYP2D6-PM phenotype and there was no significant effect of food on the PK of aficamten. Collectively, these observations support the continued development of aficamten for patients with HCM and provide a roadmap for phase 2 studies.

Safety of aficamten. No serious AEs were observed in the study and all participants completed intended dosing as planned. Generally, AEs were mild and similar in frequency between participants treated with aficamten and placebo. Importantly, there were no associated symptoms or adverse changes in vital signs for participants whose LVEF fell below 50% and the LVEF in these cases returned to baseline within 24 h. This study was not intended to find a maximum tolerated dose and hence dose escalation stopped once a clear PD effect was observed in the SAD and MAD portions of the study; thus, a dose that was not tolerated due to AEs was not identified.

Effect on LVEF. In the SAD cohorts, a dose of 50 mg produced a mean reduction in LVEF of 5.8% while in the MAD cohort, 10 mg once daily for 14 days produced a mean absolute reduction in LVEF of approximately 5%. The proportion of participants with absolute reductions in LVEF of ≥5% from baseline increased as the dose increased; up to 64% of participants in the 50-mg SAD cohort and 33% in the 10-mg MAD cohort had absolute reductions in LVEF of ≥10% from baseline. In the SAD cohorts, where the broadest range of exposures of aficamten was explored, there was a statistically significant decrease in LVEF as plasma concentrations of aficamten increased. Thus, the study achieved its secondary objective of identifying a pharmacologically active dose and describing its PK/PD relationship.

Three participants had decreases in LVEF to <50% that were rapidly reversible upon study drug discontinuation. Following a single dose of 50 mg, 2 (18%) participants experienced LVEF <50% (48.2% and 45.5%). After a single dose of 75 mg, 1 participant experienced reduction of LVEF to 34.1%. In all cases, the event was noted approximately 1.5 h after dosing, and LVEF recovered to >50% by 4 to 6 h after dosing. The SAD results informed dose selection for the other portions of the study, and there were no echocardiographic AEs in the MAD, CYP2D6-PM, or food-effect cohorts.

Implications of PK results. Aficamten demonstrated linear kinetics over the dose range of 1 mg to 50 mg; half-life was independent of concentration, and clearance was independent of dose. Steady state was achieved by the end of day 10 with the 10-mg dose and by the end of day 12 with the 5-mg and 7.5-mg doses. There was no effect of food suggestive of a need to alter dosing. These findings support once-daily dosing in either the fasted or fed state.

The relationship between plasma concentration and LVEF suggests a broad therapeutic index, which will facilitate optimization of individual doses in patients with HCM, who are expected to be titrated through an escalating range of doses until the desired PD effect is achieved. In addition, the half-life of aficamten (75 to 85 h following a single dose; 77 to 86 h following multiple doses) and observed reversibility of effect offers a potential advantage in that steady state is achieved within 2 weeks and excessive effects on LVEF are readily reversed.

Conclusions. Aficamten demonstrated a favorable safety profile in healthy participants, without serious AEs or meaningful changes in laboratory tests, ECGs, or health assessments. Any decreases in LVEF to values <50% were reversible within 6 h following single doses. Pharmacologically active doses of aficamten that may serve as starting doses for a study in patients with HCM were identified.

### Example 2

A multi-center, randomized, placebo-controlled, double-blind, dose finding phase 2 clinical trial of CK-274 in patients with symptomatic non-obstructive HCM (nHCM) or HCM with MVO was conducted. The primary objective of the trial was to determine the safety and tolerability of CK-274. The secondary objectives were to describe the concentration-response relationship of CK-274 on the resting left ventricular ejection fraction (LVEF) as measured by echocardiography during 10 weeks of treatment, to describe the dose response relationship of CK-274, and to evaluate the plasma concentrations of CK-274 in patients with nHCM.

The study excluded patients receiving disopyramide. All patients received up to three escalating doses of CK-274 based on echocardiographic guidance. Overall, the treatment duration was 10 weeks with a 4-week follow-up period after the last dose.

Since patient characteristics vary substantially in this disease, individualized dose titration to a pharmacodynamics (PD) response was conducted. This included preservation of LVEF ≥55% to maximize efficacy and safety.

Patients were eligible to be included in the study only if all the following criteria apply: 1. Able to comprehend and willing to sign an informed consent form (ICF) and willing to comply with all study procedures and restrictions for the duration; 2. Males and females between 18 and 85 years of age at screening; 3. Body weight is ≥45 kg at screening; 4. Diagnosed with HCM per the following criteria: (a) has LV hypertrophy and non-dilated LV chamber in the absence of other cardiac disease; and (b) has minimal wall thickness ≥15 mm (minimal wall thickness ≥13 mm is acceptable with a positive family history of HCM or with a known disease-causing gene mutation); 5. Adequate acoustic windows for echocardiography; 7. Left ventricular ejection fraction (LVEF) ≥60% at screening; 8. New York Heart Association (NYHA) Class II or III at screening; 9. Patients on beta-blockers, verapamil, diltiazem, or ranolazine should have been on stable doses for ≥4 weeks and anticipate remaining on the same medication regimen during the study; 10. Male patients were eligible to participate if they agreed to the following during the study and for at least 10 weeks after the last dose: (a) refrain from donating sperm; plus either (b)(i) be abstinent from heterosexual intercourse as their preferred and usual lifestyle (abstinent on a long term and persistent basis) and agree to remain abstinent; or (b)(i) must agree to use a male condom and, when his female partner is a woman of childbearing potential, have his female partner use a highly effective method of contraception; 11. A female patient was eligible to participate if she was not pregnant or breastfeeding, and at least one of the following conditions applied: (a)(i) is not a woman of childbearing potential, or (a)(ii) is a woman of childbearing potential and using a highly effective method of contraceptive during the study and for at least 4 weeks after the last dose; and (b) a woman of childbearing potential must have a negative pregnancy test (urine or serum as required by local regulations) within 3 days before the first dose of study intervention; 12. Able to complete all screening procedures.

Patients were excluded from the study if any of the following criteria applied: 1. Aortic stenosis or fixed subaortic obstruction; 2. Known infiltrative or storage disorder causing cardiac hypertrophy that mimics HCM (e.g., Noonan syndrome, Fabry disease, amyloidosis); 3. History of left ventricular (LV) systolic dysfunction (LVEF <45%) at any time during their clinical course; 4. Documented history of current obstructive coronary artery disease (>70% stenosis in one or more epicardial coronary arteries) or documented history of myocardial infarction; 6. Prior treatment with cardiotoxic agents such as doxorubicin or similar; 7. Has been treated with disopyramide or antiarrhythmic drugs that have negative inotropic activity within 4 weeks prior to screening; 8. Has any ECG abnormality considered by the investigator to pose a risk to patient safety (e.g., second degree atrioventricular block type II); 9. Paroxysmal atrial fibrillation or flutter documented during the screening period; 10. Paroxysmal or permanent atrial fibrillation requiring rhythm restoring treatment (e.g., direct-current cardioversion, ablation procedure, or antiarrhythmic therapy)<6 months prior to screening, except that this exclusion does not apply if atrial fibrillation has been treated with anticoagulation and adequately rate-controlled for ≥6 months; 11. History of syncope or sustained ventricular tachyarrhythmia with exercise within 6 months prior to screening; 12. Implantable cardioverter defibrillator (ICD) placement within 3 months prior to screening or planned ICD placement during the study; 13. History of appropriate ICD shock for life-threatening ventricular arrhythmia within six months prior to screening; 14. Recipient of a major organ transplant (e.g., heart, lung, liver, bone marrow, renal) or anticipated transplantation within 12 months from randomization); 15. Hepatic impairment defined by a total bilirubin (TBL) ≥1.5× the upper limit of normal (ULN), or alanine aminotransferase (ALT) or aspartate aminotransferase (AST) ≥3×ULN at screening, except that patients with documented Gilbert syndrome and TBL ≥1.5×ULN due to unconjugated hyperbilirubinemia, without other hepatic disease, were permitted; 16. History or evidence of any other clinically significant disorder, malignancy, active infection, other condition, or disease that, in the opinion of the investigator or the Medical Monitor, would pose a risk to patient safety or interfere with the study evaluation, procedures, or completion; 17. Hemoglobin <10.0 g/dL at screening; 18. Estimated glomerular filtration rate (eGFR)<30 mL/min/1.73 m² (by the modified Modification of Diet in Renal Disease equation) at screening; 19. Currently participating in another investigational device or drug study or received an investigational device or drug <1 month (or 5 half-lives for drugs, whichever is longer) prior to screening; 20. Has received prior treatment with or is currently receiving mavacamten; 21. Has a known hypersensitivity to any excipients in CK-3773274 tablets, film-coated (e.g., mannitol, microcrystalline cellulose, croscarmellose, hydroxypropyl cellulose, sodium lauryl sulfate, magnesium stearate, Opadry QX White 21A180025).

In the study, a patient received up to three escalating doses of CK-274 as shown in Table 11. Each patient received Dose 1 once daily for 2 weeks. At Week 2, the patient had an echocardiogram 2 hours following administration of their dose. Patients up-titrated to Dose 2 if the biplane LVEF ≥55%. Otherwise, the patient remained on Dose 1. If LVEF was <50% at Week 2, treatment was discontinued. The dose-adjustment algorithm is shown below in Table 10.

After 2 more weeks on the assigned dose (i.e., Week 4), each patient had an echocardiogram 2 hours following administration of their dose. Patients escalated to the next higher dose if the biplane LVEF ≥55%. Otherwise, the patient remained on the same dose. If LVEF was <50% at Week 4, the patient returned to a prior dose level or treatment was discontinued if the patient was on Dose 1.

After 2 more weeks on the assigned dose (i.e., Week 6), each patient had an echocardiogram 2 hours following administration of their dose. If LVEF was <50% at Week 6, the patient was down-titrated to a prior dose level or discontinued if the patient was on Dose 1.

TABLE 10
Dose Adjustment Algorithm

| Biplane LVEF | Action |
|---|---|
| <40% | Permanent discontinuation |
| <50% | Dose reduced to next lower dose, or |
| | discontinue treatment (if on lowest dose) |
| ≥50% and <55% | Dose unchanged |
| ≥55% | Dose escalated to next dose level, or |
| | dose unchanged (if on highest dose) |

TABLE 11
Dosing Scheme

| Dose 1 | Dose 2 | Dose 3 |
|---|---|---|
| 5 mg | 10 mg | 15 mg |

Echocardiograms obtained on day 1 of treatment, at weeks 2, 4, 6, and 10 of treatment, and two weeks after the last dose were analyzed for several key structural and physiologic metrics; measurements of N-terminal prohormone of brain natriuretic peptide (NT-proBNP), and cardiac troponin I were also monitored. Ambulatory cardiac monitoring was conducted one week before day 1 of treatment and at week 9.

Health Status and Health-related Quality of Life: when available, patients completed patient-reported outcome (PRO) questionnaires prior to dosing at Day 1, at Week 6, at Week 10 and at Week 12. The following instruments were be used at these visits: by Short Form 36 physical function sub-scale (SF-36-PFS), Kansas City Cardiomyopathy Questionnaire Clinical Summary Score (KCCQ-CSS), SAQ7, PGI-C (Week 10 only), and CGI (Week 10 only).

### Primary Safety Endpoints

Primary safety endpoints were: (i) patient incidence of reported AEs from first dose up to safety follow-up; (ii) patient incidence of reported SAEs from first dose up to safety follow-up; (iii) patient incidence of LVEF <50% from first dose up to safety follow-up.

Safety follow-up was defined as 4 weeks following the last dose.

### Exploratory Safety Endpoints

Exploratory safety endpoints from first dose of IP up to safety follow-up included: (i) Patient incidence of drug-related AEs, AEs leading to IP discontinuation, and AE severity; (ii) Observed values and changes from baseline in clinical laboratory data, vital signs, and ECG parameters (e.g., heart rate, PR interval, QRS interval, QT interval, and QTc interval [both Bazett's and Fridericia's corrections]); (iii) Categorical safety variables, including: (a) Number of patients (%) with LVEF <40% at each scheduled assessment point and at any time during the study; (b) Number of patients (%) in the following categories for QTc at each scheduled ECG assessment time: absolute value >450 ms, >480 ms, or ≥500 ms; increase from baseline >30 ms, >60 ms; (c) Clinically significant changes in clinical laboratory test values and ECG parameters; (d) Incidence of reported cardiac rhythm disturbances for a 1-week period at baseline prior to dosing and at Week 10 as assessed by the ambulatory cardiac monitoring device.

### Pharmacokinetic Endpoints

The secondary PK endpoints were observed Cmax and Ctrough for CK-274 during dosing, at timepoints listed in Table 12. PK was also assessed through the development of population PK model.

TABLE 12
Summary of PK and Echocardiography Time Points

| | | Echocardi- | Type of |
|---|---|---|---|
| | PK Time | ography | Echocardi- |
| Visit | Point ^{a} | Time Point | ogram |
| Screening | NA | No specific time | Full protocol |
| | | requirement | |
| Day 1 | Pre-dose and 1 hour | Pre-dose | Full protocol |
| Week 2 | Pre-dose, 0.5, 1 and 2 | 2-2.5 hours post | Truncated |
| | hours post-dose | dose | protocol |
| Week 4 | Pre-dose, 0.5, 1 and 2 | 2-2.5 hours post | Truncated |
| | hours post-dose | dose | protocol |
| Week 6 | Pre-dose, 0.5, 1 and | 2-2.5 hours post | Truncated |
| | 2 hours post-dose | dose | protocol |
| Week 10 | Pre-dose, 0.5, 1 and | 2-2.5 hours post | Full protocol |
| | 2 hours post-dose | dose | |
| Week 12 | Untimed PK at visit | No specific time | Full protocol |
| (EOS) | | requirement | |
| Early | Untimed PK at visit | No specific time | Full protocol |
| Discontinuation | | requirement | |

^{a} Visit occurred at least 14 days but no more than 17 days from the previous visit.

### Pharmacodynamic Endpoints

The secondary PD endpoints of the study were: Slope of the relationship of the plasma concentration of CK-3773274 to the change from baseline in the resting LVEF.

Echocardiography parameters were evaluated by the core laboratory at baseline and during treatment at the Weeks 2, 4, 6, and 10 and after a 2-week washout at Week 12 (follow-up visit).

### Exploratory Pharmacodynamic Endpoints

The exploratory PD endpoints included but were not limited to: (i) The relationship of plasma concentration ranges of CK-3773274 to LVEF, LVFS, global longitudinal strain (GLS); (ii) Observed values and change from baseline in the echocardiography parameters described in Table 13 and evaluated by the core laboratory during treatment at Weeks 2, 4, 6, 10, and 12; (iii) Change in NT-proBNP from baseline to Week 10; (iv) Change in hs-cTnI from baseline to Week 10; (v) Proportion of patients with LVEF ≥50% and ≥50% reduction from baseline NT-proBNP.

For select PD measurements with statistical distributions likely to depart from normal, proportional change from baseline was evaluated.

TABLE 13
Echocardiographic Parameters Measured

| | | |
|---|---|---|
| Resting LVOT-G | LVEDV | IVST |
| Post-Valsalva LVOT-G | LVESD | IVCT |
| LVEF | LVESV | IVRT |
| LVFS | LVCO | E/E′ ratio |
| GLS | LV Stroke Volume | LAV |
| LVEDD | LVOT VTI | |

GLS = global longitudinal strain;
IVCT = isovolumic contraction time;
IVRT = isovolumic relaxation time;
IVST = interventricular septum thickness;
LAV = left atrial volume;
LVCO = left ventricular cardiac output;
LVEDD = left ventricular end diastolic diameter;
LVEDV = left ventricular end diastolic volume;
LVESD = left ventricular end systolic diameter;
LVESV = left ventricular end systolic volume;
VTI = velocity time integral.

### Other Exploratory Endpoints

Other exploratory endpoints included: (i) Change in NYHA Functional Classification from baseline to Week 10; and (ii) For patients with baseline NYHA Class III disease, percentage with improvement to NYHA Class I or II disease at Week 10.

The exploratory health status and health-related quality of life endpoints included but were not limited to change from baseline to Week 10 and a slope of change from baseline to Week 10 in: (i) T-scores for PROMIS Dyspnea Severity Short Form 10a, PROMIS Dyspnea Severity Short Form 10a with two additional items, PROMIS Fatigue Short Form 7a, and PROMIS Physical Function Short Form 8b; (ii) Norm-based T scores for SF-36 Physical Functioning subscale; (iii) Individual responses to the HCM Global Impression of Status questionnaire at baseline, Week 6, and Week 10; (iv) Individual responses to the HCM Global Impression of Change questionnaire at Week 6 and Week 10; (v) Change in KCCQ score from baseline to Week 10; and (vi) Change in SAQ7 score from baseline to Week 10.

### Interim Results

41 patients were enrolled. Baseline characteristics are shown in Table 14.

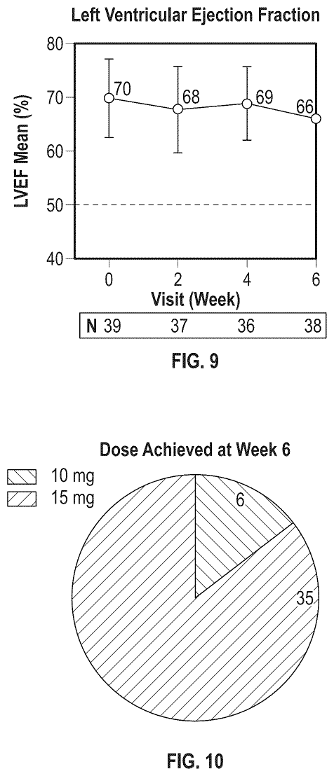

During the titration period (weeks 0-6), LVEF decreased modestly; no LVEF were less than 50% during the titration period (see FIG. 9). 35 patients (85%) achieved daily aficamten dose of 15 mg; 6 (15%) achieved 10 mg (see FIG. 10).

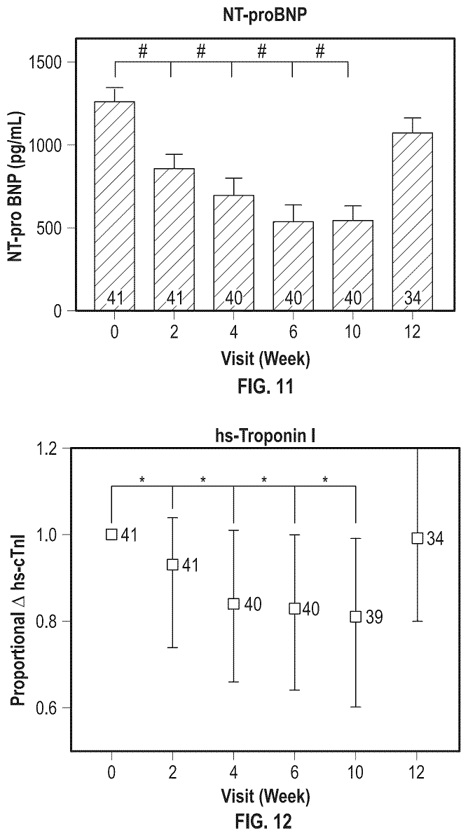

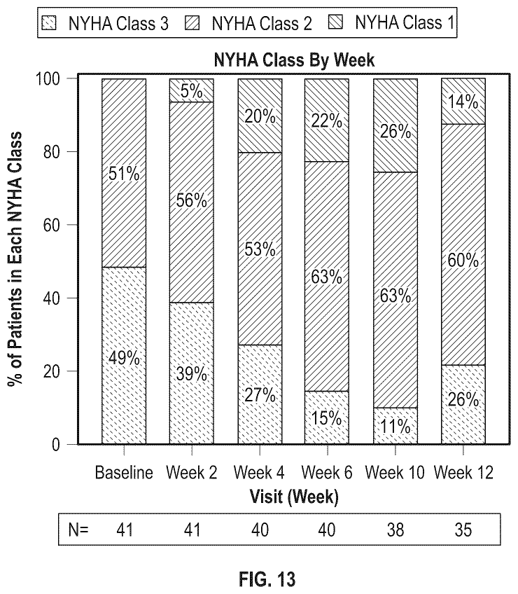

Geometric mean NT-proBNP (% CV) decreased at each scheduled visit with the proportional change from baseline being highly statistically significant (see FIG. 11, # P<0.0001); a significant reduction of 56% (p<0.0001) was achieved by end of treatment (week 10). hs-cTnI also decreased significantly proportional to baseline at each study visit (* p<0.05), and a significant reduction of 21% (p<0.05) was achieved by end of treatment (week 10); after the 2-week washout, both cardiac biomarkers rebounded upwards off drug (see FIG. 12). NYHA Class improved during treatment. 22 of 41 (54%) patients experienced a change of ≥1 NYHA Class, including 12 patients who improved from Class III to II; 4 patients who improved from Class III to I; and 8 patients who improved from Class II to I (see FIG. 13).

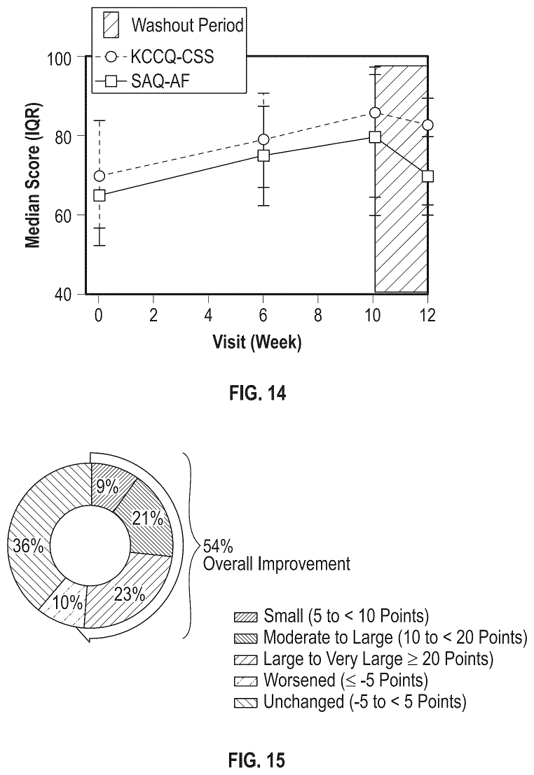

KCCQ-CSS scores improved from a baseline median (IQR) of 69.8 (56.8, 83.9) by 7.3 (−1.0, 18.8) points, and with 21 (54%) patients improving by ≥5 points (FIG. 14 and FIG. 15). The 14 (34%) patients with symptomatic angina (SAQ-AF≤80) at baseline experienced an improvement in response to treatment, with SAQ-AF increasing by a mean (SD) change of 14.3 (±16.0) from baseline to Week 10 (p=0.005) (FIG. 14).

TABLE 14
Baseline characteristics

| | N = 41 | |
|---|---|---|
| Age in Years, Mean (SD) | 55.9 | (15.8) |
| Sex, n (%) | | |
| Female | 24 | (58.5) |
| Race, n (%) | | |
| White | 28 | (68.3) |
| Black or African American | 8 | (19.5) |
| Asian | 2 | (4.9) |
| Other | 3 | (7.3) |
| BMI (kg/m²) | | |
| Mean (SD) | 30.0 | (7.1) |
| NYHA Class | | |
| Class II | 21 | (51.2) |
| Class III | 20 | (48.8) |
| LVEF (%) - Site Mean (SD) | 68.1 | (5.5) |
| NT-proBNP (pg/mL), GeoMean (% CV) | 1254 | (80.1) |
| hs-cTroponin I (ng/mL) GeoMean (% CV) | 28.7 | (317.6) |

### Interim Safety

85% of the cohort achieved dose of 15 mg by week 6 (see FIG. 10). 66% patients had at least 1 TEAE (see Table 15). There were no drug discontinuations due to AE. One patient had dose reduction to 10 mg for AE fatigue at week 9. One patient had dose interruption for 2 day due to AE palpitation (resolved). Three patients had SAEs: Bronchitis, new onset atrial fibrillation, cardiac arrest. None of these were deemed related to aficamten by the Investigator.

3 patients (7.3%) had LVEF <50% at Week 10. 2 occurred in patients with permanent atrial fibrillation, one of whom reported palpitations that required adjustment of rate-control medications. No AEs of heart failure were reported. All 3 patients returned to baseline LVEF by Week 12.

TABLE 15
Safety

| Safety Category | N = 41 (%) | |
|---|---|---|
| Patients reporting at least one TEAE | 27 | (66) |
| Patients with TESAEs | 3 | (7.3) |
| Patients with fatal TEAEs | 1 | (2.4) not related |
| Patients with TEAEs leading to drug | 1 interruption* | |
| interruption/drug | | |
| discontinuation | | |
| Patients with Severe TEAEs | 3 | (7.3) |
| Patients with Moderate TEAEs | 14 | (34) |
| Patients with AEs related to study drug | 4 | (9.8) |
| (per Investigator) | | |

*Patient self-interrupted IP for 2 days because of Palpitations (AE) in setting of Upper Respiratory Infection (AE). Patient restarted IP upon instruction from site. Palpitations resolved.

### Second Interim Results

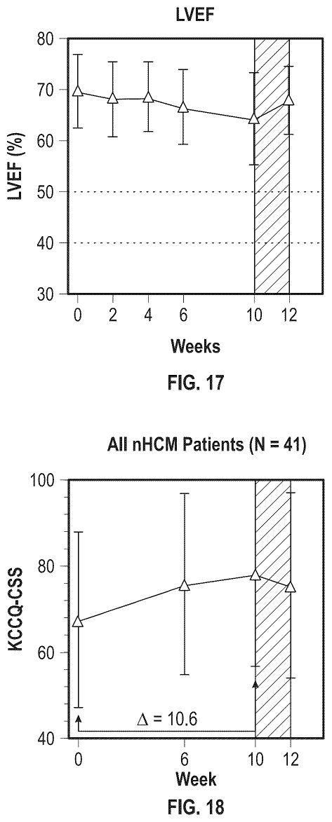

41 patients were enrolled. Baseline characteristics are shown in Table 16. There was a modest and reversible reduction in LVEF from baseline to Week 10 of −5.5% (9.9) (FIG. 17). There were no treatment interruptions or down-titration events related to LVEF <50% and no events with LVEF <40%.

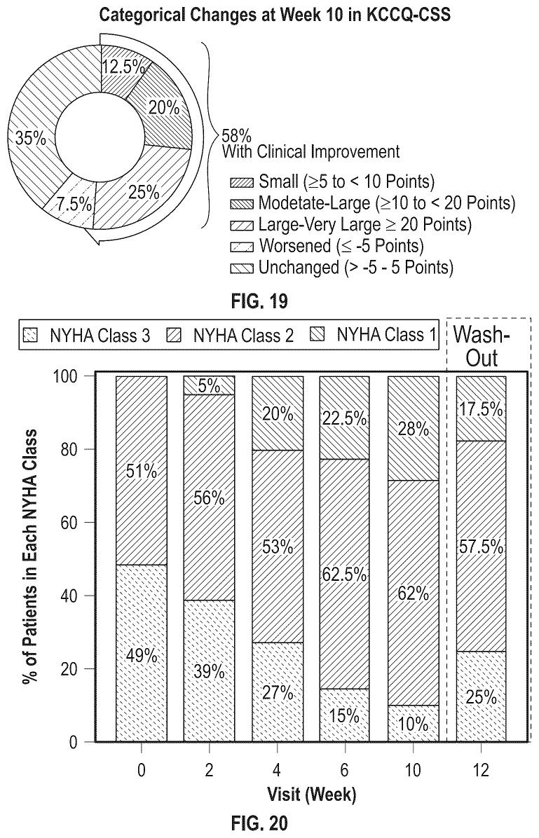

At week 10, mean KCCQ-CSS improved by 10.6 points (p<0.0001 for change from baseline to 10 weeks) (FIG. 18, data presented as mean and standard deviation). 58% of all patients had clinical reduction in symptom burden, with almost half the patients reporting moderate and large-very large improvements (FIG. 19).

NYHA Class improved during treatment. 28% of patients achieved NYHA Class 1 (asymptomatic) by Week 10 (FIG. 20), and 56% of all patients demonstrated functional improvement of ≥1 NYHA class. Change from baseline to Week 10 showed p=0.011 compared with assumed 37% placebo effect.

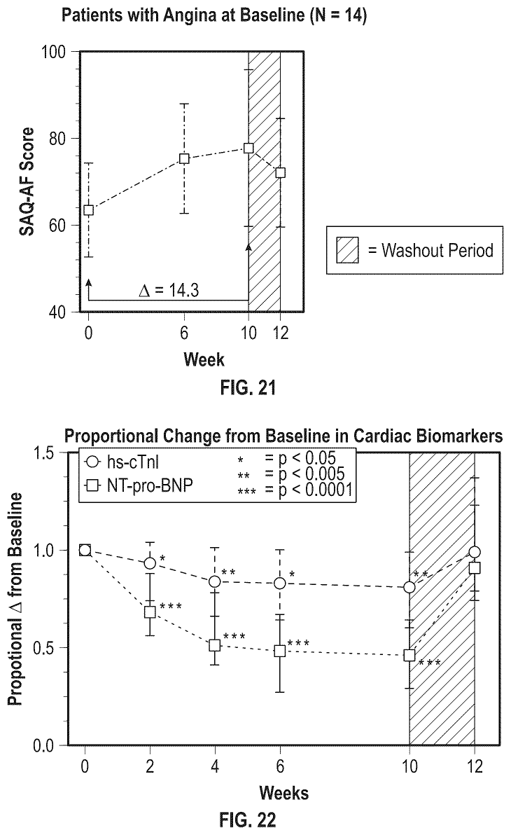

The 14 patients (34% of patients) with symptomatic angina (SAQ-AF≤80) at baseline experienced an improvement in response to treatment, with SAQ-AF increasing by a mean (SD) change of 14.3 (±16.0) from baseline to Week 10 (p=0.005) (FIG. 21).

Median NT-proBNP (IQR) decreased at each scheduled visit with the proportional change from baseline being highly statistically significant (see FIG. 22, *** P<0.0001). A significant mean (SE) reduction of −870 μg/mL (155.3), or 55% (p<0.0001), was achieved by end of treatment (week 10). Median hs-cTnI (IQR) also decreased significantly proportional to baseline at each study visit (* p<0.05; ** p<0.005) (see FIG. 22). A significant mean (SE) reduction of −24.8 ng/L (11.8), or 21% (p<0.05), was achieved by end of treatment (week 10). After the 2-week washout, both cardiac biomarkers rebounded upwards off drug (see FIG. 22).

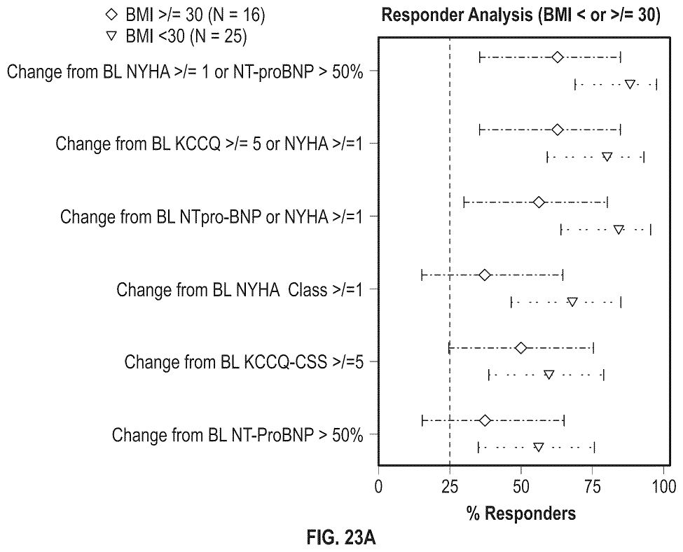

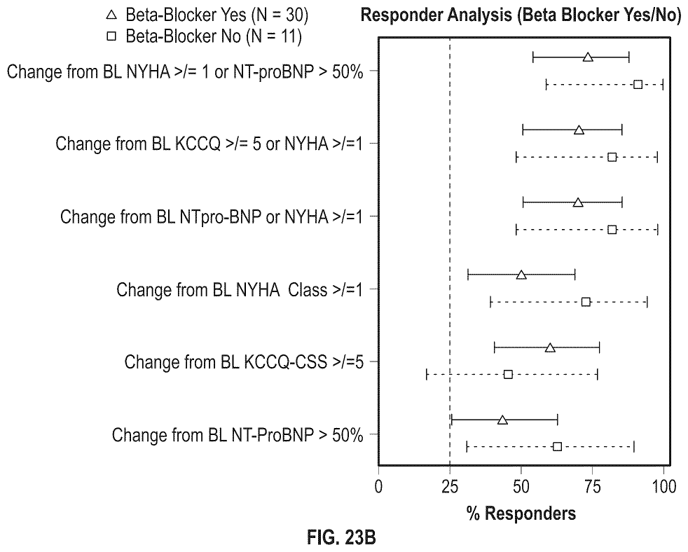

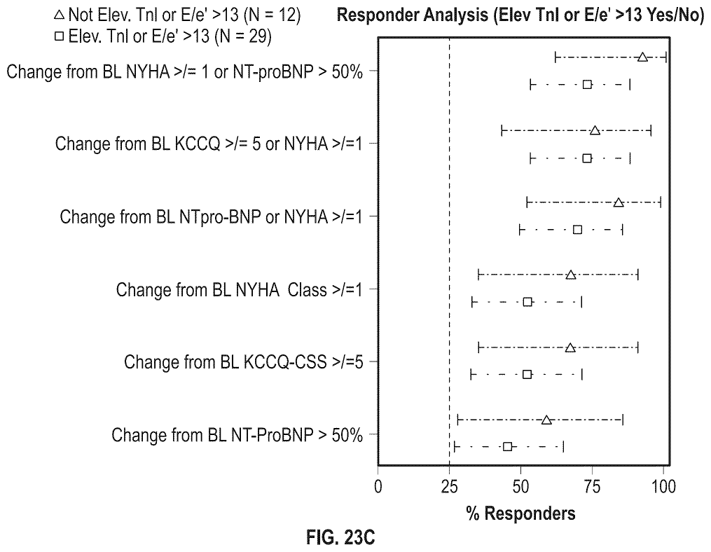

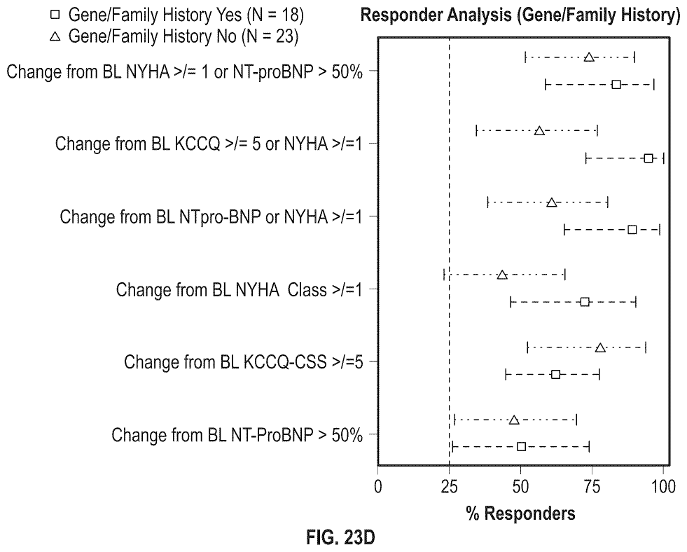

An exploratory responder analysis was performed to consider treatment effect in certain subgroups of patients. Responders were defined as either exhibiting (a) reduction in NT-proBNP >50%, (b) improvement in NYHA Class ≥1, (c) improvement in KCCQ ≥5 points, or (d) combinations of the foregoing. A potential differential treatment effect was observed in patients with BMI ≥30 compared to patients with BMI <30 (FIG. 23A). Consistent treatment effects were observed in other patient subgroups: patients receiving beta-blockers vs. patients not receiving beta-blockers (FIG. 23B); patients with elevated TnI or E/e′(>13) vs. without elevated TnI or E/e′ (FIG. 23C); patients with genetic or family history of HCM vs. patients without genetic or family history (FIG. 23D).

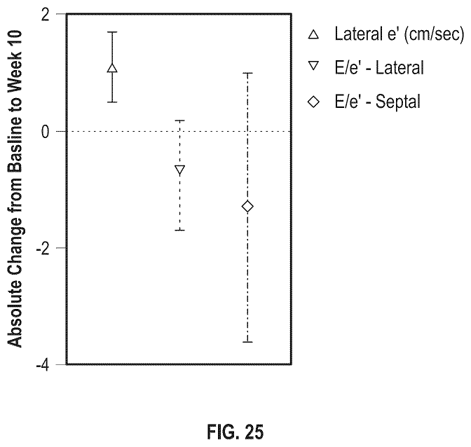

Doppler measures of diastolic function (lateral e′; lateral E/e′; septal E/e′) in patients generally improved from baseline to week 10 (FIG. 25, mean and 95% CI shown).

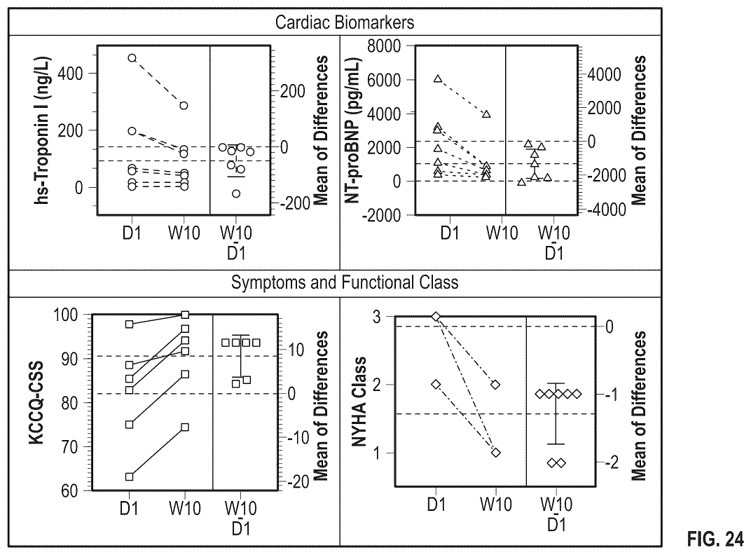

Of the 41 patients enrolled, 7 patients with mid-ventricular obstruction (MVO) were studied. Patients with MVO represent a subgroup of nHCM patients who are often excluded from other studies, but suffer substantially from limiting symptoms. All patients in this subgroup showed symptom improvement and reduction in both NT-proBNP and hs-Troponin I levels (FIG. 24).

TABLE 16
Baseline Characteristics

| | N = 41 | |
|---|---|---|
| Age in Years, Mean (SD) | 55.9 | (15.8) |
| Sex, n (%) | | |
| Female | 24 | (58.5) |
| Race, n (%) | | |
| White | 27 | (65.9) |
| Black or African American | 8 | (19.5) |
| Asian | 2 | (4.9) |
| Other | 4 | (9.8) |
| BMI (kg/m²) | | |
| Mean (SD) | 30.0 | (7.1) |
| NYHA Class | | |
| Class II | 21 | (51.2) |
| Class III | 20 | (48.8) |
| Atrial fibrillation/flutter, n (%) | 4 | (9.8) |
| NT-proBNP pg/mL, Median (IQR) | 1103 | (755-2037) |
| hs-cTroponin I ng/mL, Median (IQR) | 21.6 | (9.5-67.7) |
| LVEF, % (SD) | 69.8 | (7.2) |
| Lateral e′, mean (SD), cm/s | 7.3 | (2.9) |
| E/e′, mean (SD) | 11.6 | (5.0) |
| Left atrial volume index, mean (SD), mL/m² | 34.0 | (11.3) |
| Background HCM Therapy | | |
| Beta-blocker, n (%) | 30 | (73.2) |
| Calcium channel blocker, n (%) | 4 | (9.8) |

### Second Interim Safety

3 (7.3%) patients experienced LVEF <50% at Week 10 (EOT). No LVEF <40% occurred. All LVEF returned to normal after 2 weeks' washout (Week 12) and there were no associated SAEs. 4 (9.8%) patients experienced SAEs including one death. None of the SAEs were attributed to aficamten.

### Conclusions

Aficamten was well-tolerated overall with modest on-target reductions in LVEF in response to aficamten over 10 weeks. There was significant improvement in heart failure burden in most nonobstructive patients accompanied by improvement in cardiac biomarkers during open label therapy. Significant improvement in angina was also achieved. A mean reduction in angina frequency score of 14.3 points translates to a reduction in the frequency of angina from daily or weekly, to weekly or monthly. Trends in markers of diastolic function suggest that longer exposure at target doses may result in favorable echocardiographic evidence of improved myocardial relaxation.

### Example 3

An open-label extension clinical trial of CK-274 in patients with symptomatic nHCM or HCM with MVO is conducted. The treatment duration is anticipated to be multiple years. The primary objective of the trial is to determine the safety and tolerability of CK-274 over a 5-year period.

Approximately 25 patients may be enrolled in this study. After (up to) 56-days of screening, eligible patients are administered a daily dose of aficamten. The highest maximum tolerated dose is informed by the ongoing conduct of other studies of aficamten. Each patient starts at the lowest prespecified dose and undergoes echocardiography-guided dose titration to their maximum tolerated dose (not to exceed the highest prespecified dose). Dose adjustment may be made no more frequently than every 2 weeks.

Patients who complete the study as described in Example 2 and have not developed atrial fibrillation are eligible to enroll in the study. Echo-guided dose titration based on site reads is managed by the investigator and can occur at any time during the trial, as described below.

### Study Design

On Day 1, each patient has an echocardiogram; each patient receives Dose 1 of CK-274 once daily for 2 weeks. At Week 2, each patient has a truncated echocardiogram 2 hours following administration of their dose. Patients are up-titrated to Dose 2 if the biplane LVEF ≥55%. Otherwise, the patient remained on 5 mg of CK-274. If LVEF is <50%, treatment is discontinued.

At Weeks 4, 6, 12, and every 12 weeks thereafter, each patient has an echocardiogram or truncated echocardiogram 2 hours following administration of their dose (a truncated echocardiogram at weeks 4 and 6; and an echocardiogram at week 12 and every 12 weeks thereafter) to determine whether additional dose titration is needed (see Table 17 and Table 18).

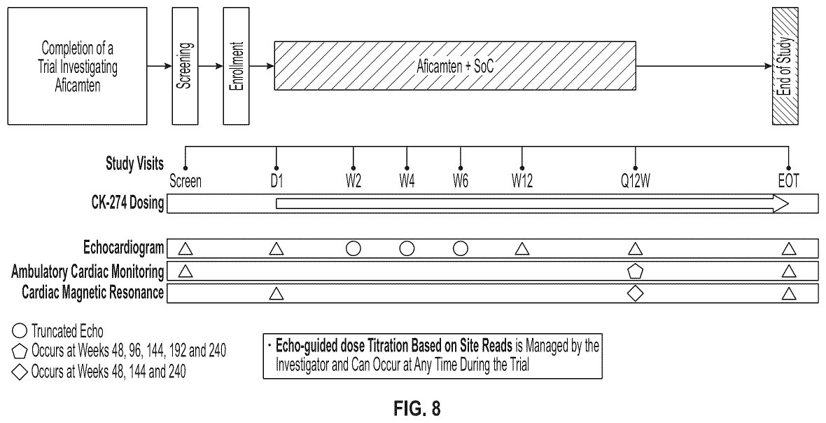

Ambulatory cardiac monitoring is performed on weeks 48, 96, 144, 192 and 240. Cardiac magnetic resonance is monitored at weeks 48, 144 and 240. (See FIG. 8). If a patient develops an LVEF <40% after the Week 2 visit, a repeat echocardiogram may be performed to confirm the initial finding (preferably within 24 hours). If the echocardiography findings are confirmed, the patient will undergo a ≥2-day drug holiday. The patient may continue CK-274 at a reduced, prior tolerated dose once a local echocardiogram documents an LVEF ≥55%.

TABLE 17
Dose Adjustment Algorithm

| Biplane LVEF | Action |
|---|---|
| <40% | Discontinuation |
| <50% | Dose reduced to next lower dose, or |
| | discontinuation if on lowest dose |
| ≥50% and <55% | Dose unchanged |
| ≥55% | Dose escalated to next dose level, or |
| | Dose unchanged if on highest dose |

TABLE 18
Dosing Scheme

| Dose 1 | Dose 2 | Dose 3 | Dose 4 |
|---|---|---|---|
| 5 mg | 10 mg | 15 mg | 20 mg |

### Echocardiography

Echocardiographic parameters to be measured at least include the left ventricular parameters (resting left ventricular outflow tract pressure gradient (LVOT-G), post-Valsalva LVOT-G, LVEF, LVFS, left ventricular strain, left ventricular end diastolic volume (LVEDV), left ventricular end systolic volume (LVESV), LV Stroke Volume), septal and free wall thickness, E/e′, E/A, LA volume.

### Cardiac Magnetic Resonance

A cardiac magnetic resonance (CMR) imaging sub-study assesses the effects of long-term administration of aficamten dosing on cardiac morphology, function, and fibrosis in those HCM patients who are eligible and elect to participate. CMR is performed at baseline any time during the screening period and may be performed within 8 weeks prior to the first dose of aficamten given on Day 1. Patients that screen fail and rescreen do not need a repeat baseline CMR. Baseline CMR does not need to be repeated if subject has enrolled in the Example 5 CMR sub-study. Patients with eGFR <30 mL/min/1.73 m2 or an allergy to gadolinium may only be given non-contrast CMR. Patients may also choose only non-contrast CMR evaluations for any other reason. Subsequent CMR studies are performed ±30 days of the Week 48 and Week 144 visits, or within 60 days prior to the Week 240 or end of treatment (EOT) visit.

NYHA Functional Classification is recorded. When available, patients will complete patient-reported outcome (PRO) questionnaires: Kansas City Cardiomyopathy Questionnaire (KCCQ), Seattle Angina Questionnaire-7 (SAQ-7), EuroQol 5-Dimension 5-Level Instrument (EQ-5D-5L), Patient's Global Impression of Change (PGI-C) Scale, Clinical Global Impressions (CGI) Scale, SF-36 Physical Functioning Subscale (SF-36 PFS) as specified in Table 19.

TABLE 19
Schedule of health status and health-related quality of life

| | Day 1 | W 12 | Q12 W | W 48 | W 96 | W 144 | W 192 | W 240 | EOT |
|---|---|---|---|---|---|---|---|---|---|
| KCCQ | X | X | X | X | X | X | X | X | X |
| SAQ-7 | X | X | X | X | X | X | X | X | X |
| EQ-5D-5L | X | X | X | X | X | X | X | X | X |
| PGI-C | | X | X | X | X | X | X | X | X |
| CGI | | X | X | X | X | X | X | X | X |
| SF-36 PFS | X | | | X | X | X | X | X | X |

### Primary Endpoints

The primary endpoints are: (i) Patient incidence of reported adverse events (AEs); (ii) Patient incidence of reported serious adverse events (SAEs); (iii) Patient incidence of left ventricular ejection fraction (LVEF)<50%.

### Exploratory Endpoints

Exploratory objectives of the study include (i) assessing steady-state pharmacokinetics during long-term administration of aficamten, as monitored by C_{trough} at 1 year intervals through end of participation; (ii) assessing long-term effects of aficamten on cardiac biomarkers, as evaluated by change from baseline values in NT-proBNP, hs-cTnI, Galectin-3, PINP, TIMP-1, CITP, Soluble ST2, and other biomarkers at 12-week intervals through end of participation; (iii) assessing effect on functional outcomes such as shift from baseline in NYHA functional class at 12-week intervals through end of participation; (iv) assessing effect on symptoms of nHCM, as evaluated by change from baseline to end of participation, in 12-week intervals, for EQ-5D-5L, PGI-C, CGI, Kansas City Cardiomyopathy Questionnaire (KCCQ), SAQ-7, and SF-36 Physical Functioning Subscale (SF-36 PFS); (v) assessing the pharmacodynamic effect of aficamten on cardiac function and structure, as evaluated by change from baseline to end of participation at 12-week intervals in the following echocardiographic cardiac function and structure measurements: LVEF; Left ventricular fractional shortening (LVFS); Left ventricular stroke volume (LVSV); Left ventricular end-systolic and end-diastolic volumes (LVESV and LVEDV, respectively); Septal, free wall, and maximal wall thickness; Left atrial volume; Left ventricular strain (longitudinal, circumferential, radial); Diastolic indices: E/e′, E/A; (vi) assessing the effect of aficamten on electrocardiographic indices of abnormal myocardial repolarization, as evaluated by change from baseline in the proportion of patients with an LV Strain pattern on ECG to the end of participation at 12-week intervals; (vii) assessing the effect of aficamten on cardiac structure, as evaluated by change from baseline to 1, 3 and 5 years and end of participation in cardiac morphology and structural measurements as assessed by cardiac magnetic resonance (CMR) imaging: RV and LV mass; Septal, free wall, and maximal wall thicknesses; Left atrial volume; End-diastolic volume (EDV); End-systolic volume (ESV); (viii) assessing the effect of aficamten on cardiac function, as evaluated by change from baseline to 1, 3 and 5 years and end of participation in biventricular function as assessed by CMR imaging: Stroke volume (SV); Ejection fraction (EF); Cardiac output (CO); (ix) assessing the effect of aficamten on cardiac fibrosis as evaluated by change from baseline to 1, 3 and 5 years and end of participation in the following CMR parameters with Late Gadolinium Enhancement (LGE): LGE mass (g), LGE mass % (as % of LV mass).

### Example 4

This is a Phase 3, multi-center, randomized, double-blind, placebo-controlled trial in participants with symptomatic nHCM. This Phase 3 trial is designed to further evaluate the effect of aficamten on quality of life, exercise capacity, heart failure symptoms, cardiac biomarkers, cardiac remodeling, and clinical outcomes. Additionally, the safety and tolerability of aficamten is assessed in participants with nHCM.

Approximately 420 eligible participants are randomized in a 1:1 ratio to aficamten or placebo. Doses of 5, 10, 15, or 20 mg aficamten or matching placebo are administered in an escalating manner using echocardiography to guide dose titration. Randomization is stratified by persistent atrial fibrillation and presence of intracavitary obstruction.

The study consists of 2 parts: Part 1 comprises Day 1 to Week 36; and Part 2, Week 36 to Week 72. All participants complete Part 1. At the end of Part 1, participants continue into Part 2 until the last randomized participant has completed Part 1 (Week 36). At that point, all remaining active study participants in Part 2 have an end of treatment visit followed by an end of study visit.

Enrollment is limited so that approximately <10% of participants have persistent or permanent atrial fibrillation, and approximately <10% of participants have a body mass index in the range of ≥35 to <40 kg/m2. Participants completing the study have an option to roll over into a long-term open label extension study.

A cardiovascular magnetic resonance imaging substudy is conducted in up to 100 participants.

Abbreviations: AE=adverse event; CGI=Clinical Global Impression; CPET=cardiopulmonary exercise testing; EQ-5D-5L=EuroQol 5-dimension 5-level instrument; hs-cTnI=high sensitivity cardiac troponin I; KCCQ=Kansas City Cardiomyopathy Questionnaire; KCCQ-CSS=KCCQ-Clinical Summary Score; KCCQ-OSS=KCCQ-Overall Summary Score; LAVI=left atrial volume index; LVEF=left ventricular ejection fraction; LVMI=left ventricular mass index; MRI=magnetic resonance imaging; nHCM=non-obstructive hypertrophic cardiomyopathy; NT-proBNP=N-terminal prohormone brain natriuretic peptide; NYHA=New York Heart Association; PGI-C=Patient Global Impression of Change; PK=pharmacokinetic(s); PRO=patient-reported outcome; pVO2=peak oxygen uptake; SAQ-7=Seattle Angina Questionnaire-7; SBP=systolic blood pressure; VAT=ventilatory anaerobic threshold; VE/VCO₂=minute ventilation/carbon dioxide production.

### Eligibility

Inclusion Criteria: Participants who meet all the following criteria at screening may be included in the trial: (1) Between 18-85 years of age. (2) Body mass index <40 kg/m². (3) Diagnosed with nHCM and has a screening echocardiogram with the following: (i) End-diastolic left ventricular (LV) wall thickness ≥15 mm in one or more myocardial segments, OR ≥13 mm in one or more wall segments and a known disease-causing gene mutation or positive family history of HCM, AND (ii) Resting LVOT-G <30 mmHg AND Valsalva LVOT-G <50 mmHg; AND (iii) LVEF ≥60%; note participants with a history of mid-cavitary or apical obstruction in the absence of LVOT obstruction are eligible. (4) NYHA class II or III. (5) Respiratory exchange ratio of ≥1.00 at screening by cardiopulmonary exercise testing (CPET) and predicted peak oxygen uptake (pVO2)<90% for age and sex. (6) KCCQ-CSS score of ≥30 and ≤85. (7) NT-proBNP of: (a) NT-pro BNP ≥300 μg/mL or NT-proBNP ≥900 μg/mL if in atrial fibrillation or atrial flutter, or (b) For Black participants, an NT-pro BNP ≥225 μg/mL or NT-proBNP ≥675 μg/mL if in atrial fibrillation or atrial flutter. (8) Hemoglobin ≥10 g/dL. (9) Participants on beta-blockers, verapamil, diltiazem, or ranolazine should have been on stable doses for ≥2 weeks prior to the baseline CPET and anticipate remaining on the same medication regimen during the study. (10) Male participants are eligible to participate if they agree to the following: a) Refrain from donating sperm during the trial plus at least 10 weeks after the last dose of IP and b) During the trial plus 4 weeks after the last dose of IP either: (i) Be abstinent from heterosexual intercourse as their preferred and usual lifestyle (abstinent on a long term and persistent basis) and agree to remain abstinent in writing, OR (ii) Must agree to use a male condom when his female partner is a woman of childbearing potential, and have his female partner use a highly effective method of contraception. (11) A female participant is eligible to participate if she is not pregnant, breastfeeding or planning to donate eggs, and at least one of the following conditions applies: a) Is not a woman of childbearing potential, or b) Is a WOCBP and using a highly effective method of contraception and male partner agrees to use a condom, during the trial and for at least 4 weeks after the last dose of IP, or c) A WOCBP must have a negative pregnancy test (urine or serum as required by local regulations) at Day 1, prior to the first dose of study IP

Exclusion Criteria: Participants who meet any of the following criteria are excluded from the trial: (1) Significant valvular heart disease (per Investigator judgment): (a) Moderate or more severe valvular aortic stenosis or fixed subaortic obstruction, (b) Moderate or more severe mitral regurgitation. (2) Known or suspected infiltrative, genetic or storage disorder causing cardiac hypertrophy that mimics nHCM (e.g., Noonan syndrome, Fabry disease, amyloidosis). (3) Known coronary artery stenosis of ≥70%. (4) History of LV systolic dysfunction (LVEF <45%) or stress cardiomyopathy. (5) Inability to exercise on a treadmill or bicycle (e.g., orthopedic limitations). (6) Documented room air oxygen saturation reading <90% at screening or history of significant chronic obstructive pulmonary disease or severe/significant pulmonary hypertension. (7) History of syncope, symptomatic ventricular arrhythmia, or sustained ventricular tachyarrhythmia with exercise within 3 months prior to screening. (8) History of resistant hypertension (persistently elevated blood pressure despite maximal doses of 3 or more classes of medications for hypertension control). (9) Screening diastolic blood pressure >100 mmHg. (10) Received prior treatment with aficamten. (11) Received treatment with mavacamten within 3 months prior to screening (must be discussed with the medical monitor prior to screening). (12) Undergone septal reduction therapy <6 months prior to screening. (13) Is being considered for or is likely to be considered for heart transplant listing or left ventricular assist device placement during the study period. (14) Paroxysmal or permanent atrial fibrillation is excluded only if: (a) rhythm restoring treatment (e.g., direct-current cardioversion, atrial fibrillation ablation procedure, or antiarrhythmic therapy) has been required <3 months prior to screening, (b) rate control and anticoagulation have not been achieved for at least 3 months prior to screening.

Exclusion criteria for cardiac MRI substudy: (1) Inability to tolerate Cardiac MRI. (2) Has an implantable cardioverter-defibrillator (ICD). (3) Has a cardiac pacemaker.

### Procedures

Participants randomized to aficamten may receive up to four escalating doses of IP over the initial 6 weeks of the trial as outlined below in Table 20 and Table 21. For participants who are randomized to aficamten, the initial dose of aficamten is 5 mg (Dose 1). Each participant receives Dose 1 once daily for 2 weeks. At the Week 2 visit, the participant has an echocardiogram approximately 2 hours following administration of their dose of investigational product (IP). Participants up-titrate to 10 mg (Dose 2) if site-read LVEF is ≥60% on echocardiogram; otherwise, the participant remains on the same dose.

After 2 more weeks (Week 4) on the assigned dose, each participant has an echocardiogram approximately 2 hours following administration of their dose of IP. Participants up-titrate to the next higher dose if LVEF is ≥60% on echocardiogram; otherwise, the participant remains on the same dose.

After 2 more weeks (Week 6) on the assigned dose, each participant has an echocardiogram approximately 2 hours following administration of their dose of IP. Participants up-titrate to the next higher dose if LVEF is ≥60% on echocardiogram; otherwise, the participant remains on the same dose.

After 2 more weeks on the assigned dose, at the Week 8 visit each participant has an echocardiogram 2 hours following administration of their IP dose to ensure the LVEF is ≥50%. If the LVEF is <50% at Week 8, the participant is down-titrated to the next lower dose, or to placebo if the participant is on 5 mg (Dose 1).

If at an echocardiogram visit at any point in the study the LVEF is <50%, then the participant is returned to a prior dose level or IP is switched to placebo if the participant is on 5 mg, in which case the participant remains on placebo for the remainder of the study. If the scheduled echocardiogram shows an LVEF of <40% at any time, then the study drug is temporarily discontinued. Treatment may be reinitiated after discussion with the medical monitor at the next lower dose level once LVEF has recovered to ≥60%.

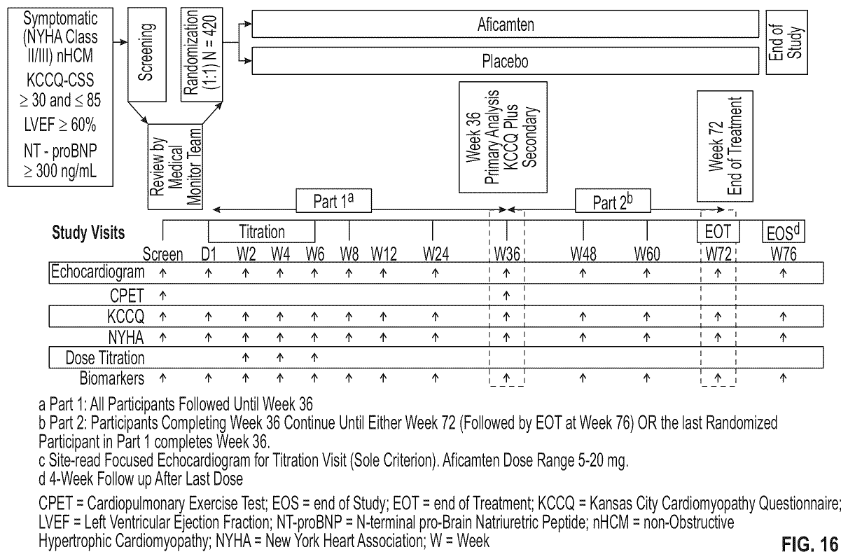

The maximum treatment duration is 72 weeks with a 4-week follow-up period after the last dose (Weeks 72 through 76). All participants are followed at minimum up through Week 36 or Part 1 of the study. Participants completing Week 36 continue in the double-blind, placebo-controlled study until either Week 72 (followed by end of treatment [EOT] at Week 76), OR until the last randomized participant in Part 1 completes Week 36. When the last randomized participant completes Week 36, all remaining active study participants have an end of treatment visit (within 4 weeks) followed by a 4-week follow-up end of study visit. A schedule of activities is shown in FIG. 16.

TABLE 20
Titration Period, Dose Titration

| | Dose 1 | Dose 2* | Dose 3* | Dose 4* |
|---|---|---|---|---|
| | (Day 1) | (Week 2) | (Week 4) | (Week 6) |
| LVEF ≥60% | 5 mg | Next higher | Next higher | Next higher |
| on | | dose, 10 | dose, 15 | dose, 20 |
| echocardiogram | | mg max | mg max | mg max |

*Echocardiogram Criteria for Scheduled Dose Titrations

TABLE 21
Echocardiogram Criteria for Scheduled Dose Titrations

| LVEF | Aficamten |
|---|---|
| ≥60% | Increase dose (Weeks 2, 4, and 6 only) |
| ≥50% to <60% | Remain on the same dose |
| <50% | Reduce dose (any visit); or down-titrate to |
| | placebo if on lowest dose (Dose 1, 5 mg) |
| <40% | Temporary discontinuation (any visit) |

TABLE 22
Key Echocardiographic LV Parameters to be measured

| | | |
|---|---|---|
| LVMI | LVEDV | IVST |
| Medial and Lateral e′ | LVESD | IVCT |
| LVEF | LVESV | IVRT |
| LVFS | LVCO | E/e′ ratio |
| | | (septal and lateral) |
| GLS | LV Stroke Volume | LAVI |
| LVEDD | LVOT VTI | Maximal LVWT |

E/e′ = ratio between early mitral inflow velocity and mitral annular early diastolic velocity;
GLS = global longitudinal strain;
IVCT = isovolumic contraction time;
IVRT = isovolumic relaxation time;
IVST = interventricular septum thickness;
LAVI = left atrial volume index;
LV = left ventricular;
LVCO = left ventricular cardiac output;
LVEDD = left ventricular end diastolic diameter;
LVEDV = left ventricular end diastolic volume;
LVEF = left ventricular ejection fraction;
LVESD = left ventricular end systolic diameter;
LVESV = left ventricular end systolic volume;
LVFS = left ventricular fractional shortening;
LVMI = left ventricular mass index;
LVOT VTI = left ventricular outflow tract velocity time integral;
LVWT = left ventricular wall thickness

TABLE 23
Schedule of health status and health-related quality of life

| | | | | | | | | | Part 2 | | | | |
|---|---|---|---|---|---|---|---|---|---|---|---|---|---|
| | Part 1 | | | | | | | | | | EOT (or | EOS (or | |
| | Day 1 | W 2 | W 4 | W 6 | W 8 | W 12 | W 24 | W 36 | W 48 | W 60 | W 72) | W 76) | ED |
| KCCQ | X | X | X | X | X | X | X | X | X | X | X | X | X |
| SAQ-7 | X | X | | | X | | | X | | | X | | X |
| EQ-5D-5L | X | X | | | X | | | X | | | X | | X |
| PGI-C | | | | | | | | X | | | X | | X |
| CGI | | | | | X | | | X | | | X | | X |
| NYHA | X | X | X | X | X | X | X | X | X | X | X | X | X |
| Functional | | | | | | | | | | | | | |
| class | | | | | | | | | | | | | |

CGI = Clinical Global Impression scale;
d = day;
ED = early discontinuation;
EOS = end of study;
EOT = end of treatment;
EQ-5D-5L = EuroQol 5-dimension 5-level instrument;
KCCQ = Kansas City Cardiomyopathy Questionnaire;
LVEF = left ventricular ejection fraction;
NYHA = New York Heart Association;
PGI-C = Patient Global Impression of Change scale;
SAQ-7 = Seattle Angina Questionnaire-7.

### Pharmacokinetics

Blood samples of approximately 4 mL are collected for measurement of plasma concentrations of aficamten as specified in Table 24. Samples are used to evaluate the PK of aficamten and potentially its metabolites

TABLE 24
PK time points

| Visit | PK Time Point |
|---|---|
| Week 2 | Pre-dose and 2 hours post dose |
| Week 4 | Pre-dose and 2 hours post dose |
| Week 8 | Pre-dose and 2 hours post dose |
| Week 36 | Pre-dose and 2 hours post dose |
| Early Discontinuation | Untimed |

### Cardiac MRI Substudy

A cardiac MRI substudy assesses the effects of administration of aficamten dosing on cardiac morphology, function, and fibrosis in up to 100 nHCM participants who are eligible and consent to participate. Cardiac MRI is performed during screening and Weeks 36 and 72. Cardiac MRI should occur after CPET if the assessments are performed on the same day. Participants with eGFR <30 mL/min/1.73 m2 or an allergy to gadolinium may have a non-contrast Cardiac MRI. Participants who screen fail and rescreen do not need a repeat Cardiac MRI.

### Statistical Methods

Assuming a difference in change from baseline in KCCQ-CSS of 5 points for aficamten compared to placebo, a standard deviation of 15, and 10% of participants missing change from baseline data of the primary endpoint, a sample size of 420 participants (210 participants per treatment group) provides more than 90% power to detect the difference in mean KCCQ-CSS change from baseline to Week 36 with a 2-sided type I error of 0.05.

During the study, the aggregate pooled SD for the change from baseline in KCCQ-CSS at Week 36 is monitored periodically in a blinded fashion. If the pooled SD is larger than expected, the sample size may be increased in order to maintain the intended power.

Efficacy analyses will be performed on the Full Analysis Set (FAS). The primary analysis tests the null hypothesis of no treatment difference in the primary endpoint between participants randomized to placebo or aficamten. Change from baseline in KCCQ-CSS is analyzed using a mixed model repeated measures model with treatment group, randomization stratification factors, visit, visit-by-baseline and visit-by-treatment group interaction as fixed effect and baseline KCCQ-CSS as covariate. A two-sample t-test is performed to compare the composite z-score between the treatment groups for the first secondary endpoint. The proportion of responders is analyzed using Cochran-Mantel-Haenszel (CMH) test stratified by randomization stratification factors. Other change from baseline endpoints are analyzed using the same model for the primary endpoint. Time-to-event endpoint is analyzed using both Kaplan-Meier estimator and Cox regression model.

Safety analyses are performed on the safety analysis set.

The number and percentage of participants reporting any treatment-emergent adverse event are tabulated by Medical Dictionary for Regulatory Activities (MedDRA) system organ class and preferred term

TABLE 25
Populations for Analyses.

| Analysis Set | Description |
|---|---|
| All Randomized Set | All randomized participants. |
| Full Analysis Set | All randomized participants who receive at least |
| (FAS) | one dose of randomized investigational product |
| | (IP) and have at least one post-baseline efficacy |
| | measurement. Participants are analyzed according |
| | to their randomized treatment group assignment. |
| | Efficacy endpoints are analyzed based on the FAS. |
| Safety Analysis Set | All randomized participants who received at least |
| | one dose of IP, aficamten or placebo. Participants |
| | are analyzed by their randomized treatment group |
| | assignment. If a participant receives treatment |
| | throughout the study that is different than the |
| | randomized treatment group assignment, then this |
| | participant is grouped by the actual treatment group |
| Pharmacokinetics | All randomized participants who have at least one |
| (PK) Analysis Set | evaluable plasma concentration of aficamten, |
| | provided they have no major protocol violations |
| | deviations that could affect the PK of aficamten. |

### Endpoints

Primary endpoint: To evaluate the effect of aficamten compared with placebo on participant health status, as measured by Change in KCCQ-CSS from baseline to Week 36.

Secondary endpoints: (1) To evaluate the effect of aficamten compared with placebo on maximal and sub-maximal exercise capacity, as measured by change in the composite of two Z-scores of CPET parameters from baseline to Week 36: pVO2 (maximal exercise capacity) and VE/VCO₂ slope (sub-maximal exercise capacity); (2) To evaluate the effect of aficamten compared with placebo on NYHA Functional Classification, as measured by proportion of participants with ≥1 class improvement in NYHA Functional Class from baseline to Week 36; (3) To evaluate the effect of aficamten compared with placebo on a biomarker of cardiac wall stress, as measured by change in NT-proBNP from baseline to Week 36; (4) To evaluate the effect of aficamten compared with placebo on echocardiographic measures of structural remodeling, as measured by change in LAVI from baseline to Week 36; (5) To evaluate the effect of aficamten compared with placebo on cardiovascular events, as measured by time to first event of cardiovascular death, heart transplantation or left ventricular assist device, aborted sudden cardiac death, non-fatal stroke, heart failure hospitalization, or cardiac arrhythmia (atrial fibrillation or ventricular tachyarrhythmia) requiring treatment or hospitalization.

Safety endpoints: To evaluate the safety and tolerability of aficamten compared with placebo in participants with nHCM, as measured by (1) Participant incidence of AEs; (2) Participant incidence of LVEF <50% in combination with: (a) Signs and symptoms of heart failure AND/OR (b) increase in NT-proBNP, relative to baseline, at the time of LVEF assessment; (3) Participant incidence of LVEF <40%.

Exploratory endpoints: (1) To evaluate the effect of aficamten compared with placebo on exercise capacity and functional class, as measured by number of participants on aficamten at Week 36 achieving either: (a) Change in pVO2 of ≥1.0 mL/kg/min from baseline, AND ≥1 class improvement in NYHA Functional Class, or (b) Change in pVO2 of ≥2.0 mL/kg/min from baseline, AND no worsening in NYHA Functional Class; (2) To evaluate the effect of aficamten compared with placebo on participant-reported health status, as measured by (a) Change in KCCQ-OSS from baseline to Week 36 and Week 72, (b) Change in KCCQ-CSS from baseline to Week 72, (c) Proportion of participants with improvement of ≥5, 10, 15 and 20 points in KCCQ-CSS and KCCQ-OSS at Week 36 and Week 72, (d) Time spent with ≥5-point improvement in KCCQ-CSS from baseline, (e) Change in SAQ-7 from baseline to Week 36 and Week 72; (3) To evaluate the effect of aficamten compared with placebo on echocardiographic measures of left ventricular structure, compliance, and filling, as measured by (a) Change in LVMI from baseline to Week 36 and Week 72, (b) Change in average medial and lateral e′ from baseline to Week 36 and Week 72; (4) To evaluate the effect of aficamten compared with placebo on echocardiographic measures of left ventricular structure, compliance, and filling, as measured by (a) Change in LVMI from baseline to Week 36 and Week 72, (b) Change in average medial and lateral e′ from baseline to Week 36 and Week 72; (5) To evaluate the effect of aficamten compared with placebo on a biomarker of myocardial injury, as measured by change in hs-cTnI from baseline to Week 36 and Week 72; (6) To evaluate the effect of aficamten compared with placebo on exercise symptoms and CPET parameters, as measured by change from baseline to Week 36 in: (a) Circulatory power (VO₂×SBP), (b) O₂ pulse, (c) VAT, (d) Total workload (watts), (e) Heart rate response, (f) Borg scale; (7) To evaluate the effect of aficamten compared with placebo on health status and health-related quality of life as measured by PRO questionnaires, as measured by change in individual responses to CGI, PGI-C, and EQ-5D-5L from baseline to Week 36 and Week 72; (8) To assess the PK of aficamten and its metabolites, as measured by pharmacokinetic parameters through Week 36; (9) To assess the effect of aficamten on myocardial remodeling as assessed by cardiac MRI, as measured by change from baseline to Week 36 and Week 72 in: (a) LVMI, (b) Septal, free wall, and maximal wall thickness, (c) Myocardial fibrosis, (d) LAVI and left atrial function, (e) Left ventricular end-systolic volume, (f) Left ventricular end-diastolic volume.

Safety analyses will be performed on the safety analysis set.

Pharmacokinetic Endpoints: Plasma concentrations of aficamten and PK parameters (maximum observed plasma concentration [Cₘₐₓ] and trough plasma concentration [C_{trough}]) are summarized using descriptive statistics including mean, SD, geometric mean, coefficient of variation, median, and range.

### Example 5

Polymorphic Form I, Form II, Form III, Form IV, Form V, and Form VI of CK-274 were characterized by various analytical techniques, including XRPD, DSC, TGA, and DVS, as described in WO 2021/011807.

1. A method of treating non-obstructive hypertrophic cardiomyopathy (nHCM) in a patient in need thereof, comprising administering to the patient a therapeutically effective amount of CK-274 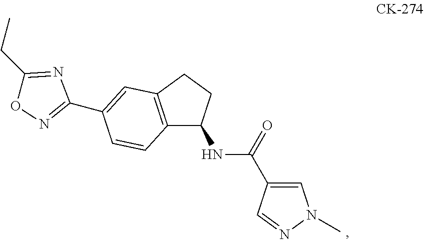 [SMILES: CCc1nc(-c2ccc3c(c2)CC[C@H]3NC(=O)c2cnn(C)c2)no1]
or a pharmaceutically acceptable salt thereof, wherein the therapeutically effective amount of CK-274, or a pharmaceutically acceptable salt thereof, is selected by titrating a daily dose of CK-274, or a pharmaceutically acceptable salt thereof, administered to the patient.

2. The method of claim 1, wherein the dose is titrated once during a course of treatment.

3-11. (canceled)

12. The method of claim 1, wherein the daily dose of CK-274, or a pharmaceutically acceptable salt thereof, is a first daily dose administered for a first time period; and wherein the method further comprises:
based on a component of a first echocardiogram for the patient acquired after the first time period, administering to the patient a second daily dose of CK-274, or a pharmaceutically acceptable salt thereof, for a second time period or terminating the administering of CK-274, or a pharmaceutically acceptable salt thereof, to the patient.

13. The method of claim 12, comprising selecting the second daily dose of CK-274, or a pharmaceutically acceptable salt thereof, based on the component of the first echocardiogram.

14-50. (canceled)

51. The method of claim 1, wherein the daily dose of CK-274, or a pharmaceutically acceptable salt thereof, is a first daily dose administered for a first time period; and wherein the method further comprises:
based a first echocardiogram comprising a biplane LVEF for the patient acquired after the first time period, administering to the patient a second daily dose of CK-274, or a pharmaceutically acceptable salt thereof, for a second time period or terminating the administering of CK-274 to the patient, wherein:
the administering of CK-274, or a pharmaceutically acceptable salt thereof, to the patient is terminated if the biplane LVEF of the first echocardiogram is below a first predetermined biplane LVEF threshold;
the second daily dose of CK-274, or a pharmaceutically acceptable salt thereof, is the same as the first daily dose of CK-274, or a pharmaceutically acceptable salt thereof, if the biplane LVEF is at or above the first predetermined biplane LVEF threshold and below a second predetermined biplane LVEF threshold; and
the second daily dose of CK-274, or a pharmaceutically acceptable salt thereof, is greater than the first daily dose of CK-274, or a pharmaceutically acceptable salt thereof, if the biplane LVEF of the first echocardiogram is above the second predetermined biplane LVEF threshold.

52. The method of claim 51, wherein the first daily dose of CK-274, or a pharmaceutically acceptable salt thereof, is about 5 mg of CK-274 and the second daily dose of CK-274, or a pharmaceutically acceptable salt thereof, is about 5 mg or about 10 mg of CK-274.

53-64. (canceled)

65. The method of claim 1, wherein the daily dose of CK-274, or a pharmaceutically acceptable salt thereof, is a first daily dose (“Dose 1”) administered for a first time period; and wherein the method further comprises:
(1) after the first time period, conducting a first echocardiogram on the patient to determine the patient's biplane LVEF (“LVEF1”); and
(a) terminating the administering of CK-274, or a pharmaceutically acceptable salt thereof, to the patient, if the patient's LVEF1 is <50%; or
(b) administering to the patient a second daily dose (“Dose 2”) of CK-274, or a pharmaceutically acceptable salt thereof, for a second time period, if the patient's LVEF1 is ≥55%, wherein Dose 2 is greater than Dose 1; or
(c) administering to the patient the same dose as the dose administered for the first time period of CK-274, or a pharmaceutically acceptable salt thereof, for a second time period, if the patient's LVEF1 is ≥50% and ≤55%; and optionally
(2) after the second time period, conducting a second echocardiogram on the patient to determine the patient's biplane LVEF (“LVEF2”); and
(a) terminating the administering of CK-274, or a pharmaceutically acceptable salt thereof, to the patient, if the patient was receiving Dose 1 during the second time period and the patient's LVEF2 is <50%; or
(b) administering to the patient Dose 1 of CK-274, or a pharmaceutically acceptable salt thereof, for a third time period, if the patient was receiving Dose 2 during the second time period and the patient's LVEF2 is <50%; or
(c) administering to the patient a greater daily dose (“Dose 2”) of CK-274, or a pharmaceutically acceptable salt thereof, for a third time period, if the patient was receiving Dose 1 during the second time period and the patient's LVEF2 is ≥55%; or
(d) administering to the patient a third daily dose (“Dose 3”) of CK-274, or a pharmaceutically acceptable salt thereof for a third time period, if the patient was receiving Dose 2 during the second time period and the patient's LVEF2 is ≥55%, wherein Dose 3 is greater than Dose 2; or
(e) administering to the patient the same dose as the dose administered during the second time period of CK-274, or a pharmaceutically acceptable salt thereof, for a third time period if the patient's LVEF2 is ≥50% and ≤55%; and optionally (3) after the third time period, conducting a third echocardiogram on the patient to determine the patient's biplane LVEF (“LVEF3”); and
(a) terminating the administering of CK-274, or a pharmaceutically acceptable salt thereof, to the patient, if the patient was receiving Dose 1 during the third time period and the patient's LVEF3 is <50%; or
(b) administering to the patient Dose 1 of CK-274, or a pharmaceutically acceptable salt thereof, for a fourth time period, if the patient was receiving Dose 2 during the third time period and the patient's LVEF3 is <50%; or
(c) administering to the patient Dose 2 of CK-274, or a pharmaceutically acceptable salt thereof, for a fourth time period, if the patient was receiving Dose 3 during the third time period and the patient's LVEF3 is <50%; or
(d) administering to the patient Dose 2 of CK-274, or a pharmaceutically acceptable salt thereof, for a fourth time period, if the patient was receiving Dose 1 during the third time period and the patient's LVEF3 is ≥55%;
(e) administering to the patient Dose 3 of CK-274, or a pharmaceutically acceptable salt thereof, for a fourth time period, if the patient was receiving Dose 2 during the third time period and the patient's LVEF3 is ≥55%;
(f) administering to the patient a fourth daily dose (“Dose 4”) of CK-274, or a pharmaceutically acceptable salt thereof for a fourth time period, if the patient was receiving Dose 3 during the third time period and the patient's LVEF3 is ≥55%, wherein Dose 4 is greater than Dose 3; or
(4) administering to the patient the same dose as the one administered during the third time period of CK-274, or a pharmaceutically acceptable salt thereof, for a fourth time period if the patient's LVEF3 is ≥50% and ≤55%.

66. The method of claim 65, wherein the first time period is about 2 weeks.

67-90. (canceled)

91. A method of treating non-obstructive hypertrophic cardiomyopathy (nHCM) in a patient in need thereof, comprising administering to the patient a therapeutically effective amount of CK-274 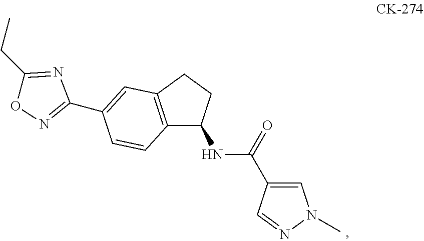 [SMILES: CCc1nc(-c2ccc3c(c2)CC[C@H]3NC(=O)c2cnn(C)c2)no1]
or a pharmaceutically acceptable salt thereof.

92. The method of claim 91, wherein the patient has a resting left ventricular ejection fraction of at least 60% prior to administering CK-274 or a pharmaceutically acceptable salt thereof.

93. The method of claim 91, wherein the patient has a resting left ventricular ejection fraction of at least 60% and resting and post-Valsalva left ventricular outflow tract pressure gradients (LVOT-G) of less than 30 mmHg prior to administering CK-274 or a pharmaceutically acceptable salt thereof.

94. The method of claim 91, wherein the therapeutically effective amount of CK-274, or a pharmaceutically acceptable salt thereof, is selected by titrating a daily dose of CK-274, or a pharmaceutically acceptable salt thereof, administered to the patient.

95. The method of claim 94, wherein the dose is titrated once during a course of treatment.

96. The method of claim 94, wherein the dose is titrated two or more times during a course of treatment.

97-111. (canceled)

112. A method of treating HCM with mid-ventricular obstruction (MVO) in a patient in need thereof, comprising administering to the patient a therapeutically effective amount of CK-274 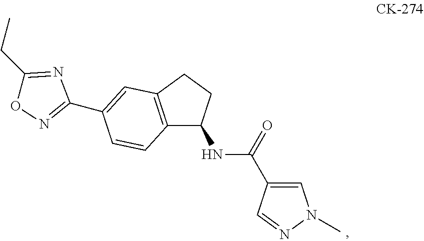 [SMILES: CCc1nc(-c2ccc3c(c2)CC[C@H]3NC(=O)c2cnn(C)c2)no1]
or a pharmaceutically acceptable salt thereof, wherein the therapeutically effective amount of CK-274, or a pharmaceutically acceptable salt thereof, is selected by titrating a daily dose of CK-274, or a pharmaceutically acceptable salt thereof, administered to the patient.

113. The method of claim 112, wherein the daily dose of CK-274, or a pharmaceutically acceptable salt thereof, is a first daily dose administered for a first time period; and wherein the method further comprises:
based on a component of a first echocardiogram for the patient acquired after the first time period, administering to the patient a second daily dose of CK-274, or a pharmaceutically acceptable salt thereof, for a second time period or terminating the administering of CK-274, or a pharmaceutically acceptable salt thereof, to the patient.

114. The method of claim 1, wherein the method results in one or more of the following for the patient:
a) improvement in NYHA functional classification by one or more classes; and/or
b) decrease in mean NT-proBNP; and/or
c) decrease in cardiac troponin I.

115-119. (canceled)

120. The method of claim 1, wherein the daily dose of CK-274, or a pharmaceutically acceptable salt thereof, is a first daily dose (“Dose 1”) administered for a first time period; and wherein the method further comprises:
(1) after the first time period, conducting a first echocardiogram on the patient to determine the patient's biplane LVEF (“LVEF1”); and
(a) terminating the administering of CK-274, or a pharmaceutically acceptable salt thereof, to the patient, if the patient's LVEF1 is <50%; or
(b) administering to the patient a second daily dose (“Dose 2”) of CK-274, or a pharmaceutically acceptable salt thereof, for a second time period, if the patient's LVEF1 is ≥60%, wherein Dose 2 is greater than Dose 1; or
(c) administering to the patient the same dose as the dose administered for the first time period of CK-274, or a pharmaceutically acceptable salt thereof, for a second time period, if the patient's LVEF1 is ≥50% and ≤60%; and optionally
(2) after the second time period, conducting a second echocardiogram on the patient to determine the patient's biplane LVEF (“LVEF2”); and
(f) terminating the administering of CK-274, or a pharmaceutically acceptable salt thereof, to the patient, if the patient was receiving Dose 1 during the second time period and the patient's LVEF2 is <50%; or
(g) administering to the patient Dose 1 of CK-274, or a pharmaceutically acceptable salt thereof, for a third time period, if the patient was receiving Dose 2 during the second time period and the patient's LVEF2 is <50%; or
(h) administering to the patient the second daily dose (“Dose 2”) of CK-274, or a pharmaceutically acceptable salt thereof, for a third time period, if the patient was receiving Dose 1 during the second time period and the patient's LVEF2 is ≥60%; or
(i) administering to the patient a third daily dose (“Dose 3”) of CK-274, or a pharmaceutically acceptable salt thereof, for a third time period, if the patient was receiving Dose 2 during the second time period and the patient's LVEF2 is ≥60%, wherein Dose 3 is greater than Dose 2; or
(j) administering to the patient the same dose as the dose administered during the second time period of CK-274, or a pharmaceutically acceptable salt thereof, for a third time period if the patient's LVEF2 is ≥50% and ≤60%; and optionally
(3) after the third time period, conducting a third echocardiogram on the patient to determine the patient's biplane LVEF (“LVEF3”); and
(a) terminating the administering of CK-274, or a pharmaceutically acceptable salt thereof, to the patient, if the patient was receiving Dose 1 during the third time period and the patient's LVEF3 is <50%; or
(g) administering to the patient Dose 1 of CK-274, or a pharmaceutically acceptable salt thereof, for a fourth time period, if the patient was receiving Dose 2 during the third time period and the patient's LVEF3 is <50%; or
(h) administering to the patient Dose 2 of CK-274, or a pharmaceutically acceptable salt thereof, for a fourth time period, if the patient was receiving Dose 3 during the third time period and the patient's LVEF3 is <50%; or
(i) administering to the patient Dose 2 of CK-274, or a pharmaceutically acceptable salt thereof, for a fourth time period, if the patient was receiving Dose 1 during the third time period and the patient's LVEF3 is ≥60%;
(j) administering to the patient Dose 3 of CK-274, or a pharmaceutically acceptable salt thereof, for a fourth time period, if the patient was receiving Dose 2 during the third time period and the patient's LVEF3 is ≥60%;
(k) administering to the patient a fourth daily dose (“Dose 4”) of CK-274, or a pharmaceutically acceptable salt thereof for a fourth time period, if the patient was receiving Dose 3 during the third time period and the patient's LVEF3 is ≥60%, wherein Dose 4 is greater than Dose 3; or
(4) administering to the patient the same dose as the one administered during the third time period of CK-274, or a pharmaceutically acceptable salt thereof, for a fourth time period if the patient's LVEF3 is ≥50% and ≤60%.

121. The method of claim 120, wherein the first time period is about 2 weeks.

122-161. (canceled)

162. The method of claim 1, wherein the CK-274 or pharmaceutically acceptable salt thereof comprises one or more of polymorphic Form I, Form II, Form III, Form IV, Form V, and Form VI of CK-274.
